(19) United States
(12) Patent Application Publication
(10) Pub. No.: US 2022/0281922 A1
(43) Pub. Date: Sep. 8, 2022

(54) AAV VARIANTS WITH ENHANCED TROPISM

(71) Applicants: Voyager Therapeutics, Inc., Cambridge, MA (US); California Institute of Technology, Pasadena, CA (US)

(72) Inventors: Dinah Wen-Yee SAH, Hopkinton, MA (US); Jinzhao HOU, Cambridge, MA (US); Kei ADACHI, Portland, OR (US); Wei WANG, Arlington, MA (US); Qingmin CHEN, Belmont, MA (US); Jenna CARROLL SOPER, Winchester, MA (US); Amy REN, Cambridge, MA (US); Xiao-Qin REN, Cambridge, MA (US); Benjamin E. DEVERMAN, Somerville, MA (US); Viviana GRADINARU, Pasadena, CA (US); Qin HUANG, Arlington, MA (US); Sripriya RAVINDRA KUMAR, Seirra Madre, CA (US)

(21) Appl. No.: 17/631,737

(22) PCT Filed: Jul. 31, 2020
(86) PCT No.: PCT/US2020/044487

§ 371 Date: Jan. 31, 2022

Related U.S. Application Data
(60) Provisional application No. 62/882,025, filed on Aug. 2, 2019.

(51) Int. Cl. C07K 14/005; C12N 15/86

(52) U.S. Cl. CPC C07K 14/005; C12N 15/86; C12N 2750/14122; C12N 2750/14123; C12N 2750/14145; C12N 2750/14171

(57) ABSTRACT

The disclosure relates to compositions, methods, and processes for the preparation, use, and/or formulation of adeno-associated virus capsid proteins, wherein the capsid proteins comprise targeting peptide inserts for enhanced tropism to a target tissue.

### CROSS REFERENCE TO RELATED APPLICATIONS

This application claims the benefit of U.S. Provisional Patent Application No. 62/882,025 filed Aug. 2, 2019, entitled “AAV VARIANTS WITH ENHANCED TROPISM”, the contents of which are herein incorporated by reference in their entirety.

### SEQUENCE LISTING

The instant application contains a Sequence Listing which has been filed electronically in ASCII format and is hereby incorporated by reference in its entirety. Said ASCII copy, created on Jul. 31, 2020 named 2057_1089PCT_SL.txt and is 11,127,739 bytes in size.

### FIELD OF THE DISCLOSURE

The disclosure relates to compositions, methods, and processes for the preparation, use, and/or formulation of adeno-associated virus capsid proteins, wherein the capsid proteins comprise targeting peptide inserts for enhanced tropism to a target tissue.

### BACKGROUND OF THE DISCLOSURE

Recombinant adeno-associated viral vectors (AAV) have proven to be a useful biological tool and are increasingly showing promise in the field of gene therapy for the treatment of human disease.

AAV-based therapies are currently being tested for the treatment of a wide-range of human disease. In some cases, the AAV-based therapy may be brought easily into contact with the system in need, such as for treating diseases of the blood (e.g., lipoprotein lipase deficiency or hemophilias), the eye (e.g., Leber's congenital amaurosis or inherited retinal diseases), or muscle (e.g., Duchenne muscular dystrophy). In other cases, such as, for example, in treating disorders of the central nervous systems (CNS; i.e., brain and spinal cord), delivery of the AAV-based therapy becomes more complicated. Direct administration generally involves invasive surgeries, while the blood brain barrier impedes access of the AAV-based therapy to the CNS if administered systemically. Further, high doses of AAV-based therapies are necessary to yield sufficient transduction of target CNS tissue, giving rise to enhanced risk of side effects and/or production difficulties given the high volumes needed.

Though the peripheral nervous system (PNS; i.e., nervous tissue outside the brain and spinal cord) may be thought of as more accessible for therapeutic intervention, some PNS tissues, such as dorsal root ganglia remain difficult to target.

To identify AAV capsid proteins with desired tropism profiles, libraries of novel capsids have been created and screened. A variety of capsid engineering methods have been used, including DNA barcoding, directed evolution, random peptide insertions, and capsid shuffling and/or chimeras. In one such method known as CREATE, a series of targeting peptides was identified that enhanced the AAV capsid protein tropism for CNS tissues. The majority of this work has been carried out in mouse models, meaning that there still remains an issue of translatability to human subjects and conditions.

Even though such ongoing efforts to generate AAV capsids with enhanced tropism to CNS tissues and/or blood brain barrier permeability have yielded improvements, a need still exists for AAV particles with enhanced tropism for CNS or PNS tissue for the use of treating human disease. Even more so, a need exists for AAV particles with enhanced tropism for CNS or PNS tissues, which may be delivered intravenously to a subject in need, at a low dose, and yet still provide sufficient transduction of the target cell-type, tissue and/or organ.

The present disclosure addresses this need by providing novel targeting peptides and associated AAV capsid proteins and AAV particles for enhanced tropism to the human CNS or PNS following intravenous delivery. In addition, the viral genomes of these AAV particles may be manipulated to suit the needs of a wide variety of CNS or PNS-associated disorders, or neurological disease.

### SUMMARY OF THE DISCLOSURE

In one aspect, the disclosure provides targeting peptides that include at least 4 contiguous amino acids of any member of a group of sequences that include but are not limited to SEQ ID NO: 43073-43341. In some embodiments, the targeting peptide may include at least 7 contiguous amino acids of any member of the group of sequences that include but are not limited to SEQ ID NO: 43073-43341.

In one aspect, targeting peptide sequences may be inserted into a parent sequence, for example, a targeting peptide may be inserted into a parent AAV capsid protein comprising a parent VP1 amino acid sequence set forth as SEQ ID NO: 2 or SEQ ID NO: 3. The parent VP1 amino acid sequence may include a VP2 region and/or a VP3 region. Targeting peptides may be inserted into the parent sequence in any of the VP1, VP2, or VP3 regions.

In one aspect, a targeting peptide may be inserted at any amino acid position between amino acids 586-592, inclusive, of the parent VP1 amino acid sequence.

In one aspect, a targeting peptide may be inserted between amino acids 588-589 of the parent amino acid sequence.

In one aspect, the disclosure provides AAV particles comprising an AAV capsid protein with a targeting sequence insert and a viral genome. The viral genome comprises a nucleic acid sequence encoding a payload, wherein the payload may be an RNAi agent or a polypeptide.

In one aspect, the AAV particles of the disclosure comprise a viral genome encoding an RNAi agent payload. The RNAi agent may be, but is not limited to, a dsRNA, siRNA, shRNA, pre-miRNA, pri-miRNA, miRNA, stRNA, lncRNA, piRNA, or snoRNA. When the RNAi agent is expressed, it inhibits or suppresses the expression of a gene of interest in a cell, wherein the gene of interest may be, but is not limited to, SOD1, MAPT, APOE, HTT, C9ORF72, TDP-43, APP, BACE, SNCA, ATXN1, ATXN2, ATXN3, ATXN7, SCN1A-SCN5A, or SCN8A-SCN11A.

In one aspect, the AAV particles of the disclosure comprise a viral genome encoding a polypeptide payload. The polypeptide may be, but is not limited to, an antibody, aromatic L-amino acid decarboxylase (AADC), survival motor neuron 1 (SMNI1), frataxin (FXN), ApoE2, GBA1, GRN, ASPA, CLN2, GLB1, SGSH, NAGLU, IDS, NPC1, or GAN.

In one aspect, the AAV particles of the disclosure may be formulated with a pharmaceutically acceptable excipient to prepare a pharmaceutical composition. The pharmaceutical composition may be administered to a subject in need in order to treat a disease in said subject. The disease may be, but is not limited to, Huntington's Disease, Amyotrophic Lateral Sclerosis, Friedreich's Ataxia, Parkinson's Disease, Alzheimer's Disease, a tauopathy or neuropathic pain.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. 1 shows a schematic for a non-limiting example of the CREATE method in non-human primate.

### DETAILED DESCRIPTION

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### I. Introduction

According to the present disclosure. AAV particles with enhanced tropism for a target tissue (e.g., CNS) are provided, as well as associated processes for their targeting, preparation, formulation and use. Targeting peptides and nucleic acid sequences encoding the targeting peptides are provided. These targeting peptides may be inserted into an AAV capsid protein sequence to alter tropism to a particular cell-type, tissue, organ or organism, in vivo, ex vivo or in vitro.

As used herein, an “AAV particle” or “AAV vector” comprises a capsid protein and a viral genome, wherein the viral genome comprises at least one payload region and at least one inverted terminal repeat (ITR). The AAV particle and/or its component capsid and viral genome may be engineered to alter tropism to a particular cell-type, tissue, organ or organism.

As used herein, “viral genome” or “vector genome” refers to the nucleic acid sequence(s) encapsulated in an AAV particle. A viral genome comprises a nucleic acid sequence with at least one payload region encoding a payload and at least one ITR.

As used herein, a “payload region” is any nucleic acid molecule which encodes one or more “payloads” of the disclosure. As non-limiting examples, a payload region may be a nucleic acid sequence encoding a payload comprising an RNAi agent or a polypeptide.

As used herein, a “targeting peptide” refers to a peptide of 3-20 amino acids in length. These targeting peptides may be inserted into, or attached to, a parent amino acid sequence to alter the characteristics (e.g., tropism) of the parent protein. As a non-limiting example, the targeting peptide can be inserted into an AAV capsid sequence for enhanced targeting to a desired cell-type, tissue, organ or organism.

The AAV particles and payloads of the disclosure may be delivered to one or more target cells, tissues, organs, or organisms. In a preferred embodiment, the AAV particles of the disclosure demonstrate enhanced tropism for a target cell type, tissue or organ. As a non-limiting example, the AAV particle may have enhanced tropism for cells and tissues of the central or peripheral nervous systems (CNS and PNS, respectively). The AAV particles of the disclosure may, in addition, or alternatively, have decreased tropism for an undesired target cell-type, tissue or organ.

### II. Compositions of the Disclosure

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

The wild-type AAV vector genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In other words, VP1 is the full length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In certain embodiments, AAV particles of the present disclosure are recombinant AAV viral vectors which are replication defective and lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ, or an organism.

In certain embodiments, the viral genome of the AAV particles of the present disclosure comprises at least one control element which provides for the replication, transcription, and translation of a coding sequence encoded therein. Not all of the control elements need always be present as long as the coding sequence is capable of being replicated, transcribed, and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV vectors of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the transduced cell.

In certain embodiments, the AAV particle of the present disclosure is an scAAV.

In certain embodiments, the AAV particle of the present disclosure is an ssAAV.

Methods for producing and/or modifying AAV particles are disclosed in the art such as pseudotyped AAV vectors (PCT Patent Publication Nos. WO200028004; WO200123001: WO2004112727; WO2005005610; and WO2005072364, the content of each of which is incorporated herein by reference in its entirety).

AAV particles may be modified to enhance the efficiency of delivery. Such modified AAV particles can be packaged efficiently and be used to successfully infect the target cells at high frequency and with minimal toxicity. In some embodiments, the capsids of the AAV particles are engineered according to the methods described in US Publication Number US20130195801, the contents of which are incorporated herein by reference in their entirety.

In certain embodiments, the AAV particles of the disclosure comprising a capsid with an inserted targeting peptide and a viral genome, may have enhanced tropism for a cell-type or tissue of the human CNS.

### AAV Capsids

AAV particles of the present disclosure may comprise or be derived from any natural or recombinant AAV serotype. AAV serotypes may differ in characteristics such as, but not limited to, packaging, tropism, transduction and immunogenic profiles. While not wishing to be bound by theory, the AAV capsid protein is often considered to be the driver of AAV particle tropism to a particular tissue.

In certain embodiments, an AAV particle may have a capsid protein and ITR sequences derived from the same parent serotype (e.g., AAV2 capsid and AAV2 ITRs). In another embodiment, the AAV particle may be a pseudo-typed AAV particle, wherein the capsid protein and ITR sequences are derived from different parent serotypes (e.g., AAV9 capsid and AAV2 ITRs; AAV2/9).

The AAV particles of the present disclosure may comprise an AAV capsid protein with a targeting peptide inserted into the parent sequence. The parent capsid or serotype may comprise or be derived from any natural or recombinant AAV serotype. As used herein, a “parent” sequence is a nucleotide or amino acid sequence into which a targeting sequence is inserted (i.e., nucleotide insertion into nucleic acid sequence or amino acid sequence insertion into amino acid sequence).

In a preferred embodiment, the parent AAV capsid nucleotide sequence is as set forth in SEQ ID NO: 1.

In another embodiment, the parent AAV capsid nucleotide sequence is a K449R variant of SEQ ID NO: 1, wherein the codon encoding a lysine (e.g., AAA or AAG) at position 449 in the amino acid sequence (nucleotides 1345-1347) is exchanged for one encoding an arginine (CGT, CGC, CGA, CGG, AGA, AGG). The K449R variant has the same function as wild-type AAV9.

In a preferred embodiment, the parent AAV capsid amino acid sequence is as set forth in SEQ ID NO: 2.

In another embodiment, the parent AAV capsid amino acid sequence is as set forth in SEQ ID NO: 3.

In certain embodiments the parent AAV capsid sequence is any of those shown in Table 1.

TABLE 1
AAV Capsid Sequences

| | SEQ ID | |
|---|---|---|
| Serotype | NO | Reference Information |
| AAV9/hu.14 (nt) | 1 | US7906111 SEQ ID NO: 3; WO2015038958 SEQ ID NO: 11 |
| AAV9/hu.14 (aa) | 2 | US7906111 SEQ ID NO: 123; WO2015038958 SEQ ID NO: 2 |
| AAV9/hu.14 K449R (aa) | 3 | WO2017100671 SEQ ID NO: 45 |

Each of the patents, applications and or publications listed in Table 1 are hereby incorporated by reference in their entirety.

The parent AAV serotype and associated capsid sequence may be any of those known in the art. Non-limiting examples of such AAV serotypes include, AAV9, AAV9 K449R (or K449R AAV9), AAV1, AAVrh10. AAV-DJ, AAV-DJ8, AAV5, AAVPHP.B (PHP.B), AAVPHP.A (PHP.A). AAVG2B-26, AAVG2B-13, AAVTH1.1-32. AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP. AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11. AAV9.13, AAV9.16, AAV9.24, AAV9.45, AAV9.47, AAV9.61, AAV9.68, AAV9.84, AAV9.9. AAV10, AAV11, AAV12, AAV16.3. AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1. AAV223.2. AAV223.4. AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54. AAV4-19/rh.55, AAV5-3/rh.57, AAV5-22/rh.58, AAV7.3/hu.7, AAV16.8/hu.10, AAV16.12/hu.11. AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15. AAV33.8/hu.16, AAV52/hu.19, AAV52.1/hu.20, AAV58.2/hu.25, AAVA3.3, AAVA3.4, AAVA3.5, AAVA3.7, AAVC1, AAVC2. AAVC5. AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21. AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51. AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8. AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37. AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74. AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31. AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2. AAV Shuffle 100-1, AAV Shuffle 100-3. AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19. AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5. AAV CHt-6.6, AAV CHt-6.7, AAV CHt-6.8, AAV CHt-P1, AAV CHt-P2. AAV CHt-P5, AAV CHt-P6, AAV CHt-P8, AAV CHt-P9, AAV CKd-1, AAV CKd-10, AAV CKd-2, AAV CKd-3, AAV CKd-4. AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv-1, AAV Clv1-10. AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6. AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5. AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11. AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the serotype may be AAVDJ or a variant thereof, such as AAVDJ8 (or AAV-DJ8), as described by Grimm et al. (Journal of Virology 82(12): 5887-5911 (2008), US Publication US20140359799 and U.S. Pat. No. 7,588,772, each of which is herein incorporated by reference in its entirety). The amino acid sequence of AAVDJ8 may comprise two or more mutations in order to remove the heparin binding domain (HBD). As a non-limiting example, the AAV-DJ sequence is as described by SEQ ID NO: 1 in U.S. Pat. No. 7,588,772, the contents of which are herein incorporated by reference in their entirety, and the AAVDJ8 sequence may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, the AAVDJ8 sequence may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In certain embodiments, the parent AAV capsid sequence comprises an AAV9 sequence.

In certain embodiments, the parent AAV capsid sequence comprises an K449R AAV9 sequence.

In certain embodiments, the parent AAV capsid sequence comprises an AAVDJ sequence.

In certain embodiments, the parent AAV capsid sequence comprises an AAVDJ8 sequence.

In certain embodiments, the parent AAV capsid sequence comprises an AAVrh10 sequence.

In certain embodiments, the parent AAV capsid sequence comprises an AAV1 sequence.

In certain embodiments, the parent AAV capsid sequence comprises an AAV5 sequence.

While not wishing to be bound by theory, it is understood that a parent AAV capsid sequence comprises a VP1 region. In certain embodiments, a parent AAV capsid sequence comprises a VP1, VP2 and/or VP3 region, or any combination thereof. A parent VP1 sequence may be considered synonymous with a parent AAV capsid sequence.

In certain embodiments, the initiation codon for translation of the AAV VP1 capsid protein may be CTG, TTG, or GTG as described in U.S. Pat. No. 8,163,543, the contents of which are herein incorporated by reference in their entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As anon-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence.

As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1-), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1-).

In certain embodiments, the parent AAV capsid sequence may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above.

In certain embodiments, the parent AAV capsid sequence may be encoded by a nucleotide sequence with 50%, 51%, 52%, 53%, 54%, 55%, 56%, 57%, 58%, 590%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described above.

In certain embodiments, the parent sequence is not an AAV capsid sequence and is instead a different vector (e.g., lentivirus, plasmid, etc.). In another embodiment, the parent sequence is a delivery vehicle (e.g., a nanoparticle) and the targeting peptide is attached thereto.

### Capsid Engineering

Recombinant or engineered AAV vectors have shown promise for use in therapy for the treatment of human disease. However, a need still exists for AAV particles with more specific and/or enhanced tropism for target tissues. Capsid engineering methods have been used to try to identify capsids with enhanced transduction of target tissues (e.g., brain, spinal cord, DRG). A variety of methods have been used, including mutational methods, DNA barcoding, directed evolution, random peptide insertions, and capsid shuffling and/or chimeras.

Rational engineering and mutational methods have been used to direct AAV to a target tissue. In rational design, structure-function relationships are used to determine regions in which changes to the capsid sequence may be made. As non-limiting examples, surface loop structures, receptor binding sites, and/or heparin binding sites may be mutated, or otherwise altered, for rational design of recombinant AAV capsids for enhanced targeting to a target tissue. In one example of rational design, AAV capsids were modified by mutation of surface exposed tyrosines to phenylalanine, in order to evade ubiquitination, reduce proteasomal degradation and allow for increased AAV particle and viral genome expression (Lochrie M A, et al, J Virol. 2006 January; 80(2):821-34; Santiago-Ortiz J L and Schaffer D V, J Control Release. 2016 Oct. 28; 240:287-301, the contents of each of which are incorporated by reference in their entirety). Rational design also encompasses the addition of targeting peptides to a parent AAV capsid sequence, wherein the targeting peptide may have an affinity for a receptor of interest within a target tissue.

In certain embodiments, rational engineering and/or mutational methods are used to identify AAV capsids and/or targeting peptides having enhanced transduction of a target tissue (e.g., CNS or PNS).

Capsid shuffling, and/or chimeras describe a method in which fragments of at least two parent AAV capsids are combined to generate a new recombinant capsid protein. The number of parent AAV capsids used may be 2-20, or more than 20.

In certain embodiments, capsid shuffling is used to identify AAV capsids and/or targeting peptides having enhanced transduction of a target tissue (e.g., CNS or PNS).

Directed evolution involves the generation of AAV capsid libraries (˜10⁴-10⁸) by any of a variety of mutagenesis techniques and selection of lead candidates based on response to selective pressure by properties of interest (e.g., tropism). Directed evolution of AAV capsids allows for positive selection from a pool of diverse mutants without necessitating extensive prior characterization of the mutant library. Directed evolution libraries may be generated by any molecular biology technique known in the art, and may include, DNA shuffling, random point mutagenesis, insertional mutagenesis (e.g., targeting peptides), random peptide insertions, or ancestral reconstructions. AAV capsid libraries may be subjected to more than one round of selection using directed evolution for further optimization. Directed evolution methods are most commonly used to identify AAV capsid proteins with enhanced transduction of a target tissue. Capsids with enhanced transduction of a target tissue have been identified for the targeting of human airway epithelium, neural stem cells, human pluripotent stem cells, retinal cells, and other in vitro and in vivo cells.

In certain embodiments directed evolution methods are used to identify AAV capsids and/or targeting peptides having enhanced transduction of a target tissue (e.g., CNS or PNS).

One method described for high-throughput characterization of the phenotypes of a large number of AAV serotypes is known as AAV Barcode-Seq (Adachi K et al, Nature Communications 5:3075 (2014), the contents of which are herein incorporated by reference in their entirety). In this next-generation sequence (NGS) based method. AAV libraries are created comprising DNA barcode tags, which can be assessed by multi-plexed Illumina barcode sequencing. This method can be used to identify AAV variants with altered receptor binding, tropism, neutralization and or blood clearance as compared to wild-type or non-variant sequences. Amino acids of the AAV capsid that are important to these functions can also be identified in this manner.

As described in Adachi et al 2014, AAV capsid libraries were generated, wherein each mutant carried a wild-type AAV2 rep gene and an AAV cap gene derived from a series of variants or mutants, and a pair of left and right 12-nucleotide long DNA bar-codes downstream of an AAV2 polyadenylation signal (pA). In this manner, 7 different DNA barcode AAV capsid libraries were generated. Capsid libraries were then provided to mice. At a pre-set timepoint, samples were collected, DNA extracted and PCR-amplified using AAV-clone specific virus bar codes and sample-specific bar code attached PCR primers. All the virus barcode PCR amplicons were Illumina sequenced and converted to raw sequence read number data by a computational algorithm. The core of the Barcode-Seq approach is a 96-nucleotide cassette comprising the DNA bar-codes (left and right) described above, three PCR primer binding sites and two restriction enzyme sites. As an exemplar, an AAV rep-cap genome was used, but the system can be applied to any AAV viral genome, including one devoid of rep and cap genes. The advantage of the Barcode Seq method is the collection of a large data set and correlation to desirable phenotype with few replicates and in a short period of time.

The DNA Barcode Seq method can be similarly applied to RNA.

In certain embodiments, the Barcode Seq method is used to identify AAV capsids and/or targeting peptides having enhanced transduction of a target tissue (e.g., CNS or PNS).

One method used to generate AAV particles with desirable transduction profiles, with enhanced targeting to CNS or PNS tissues after intravenous administration, is through the use of insertion of targeting peptides into a parent AAV capsid sequence.

### Targeting Peptides

Disclosed herein are targeting peptides and associated AAV particles comprising a capsid protein with one or more targeting peptide inserts, for enhanced or improved transduction of a target tissue (e.g., cells of the CNS or PNS).

In certain embodiments, the targeting peptide may direct an AAV particle to a cell or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, temporal), thalamus, hypothalamus, striatum, putamen, caudate nucleus, hippocampus, entorhinal cortex, basal ganglia, or deep cerebellar nuclei.

In certain embodiments, the targeting peptide may direct an AAV particle to a cell or tissue of the PNS. The cell or tissue of the PNS may be, but is not limited to, a dorsal root ganglion (DRG).

The targeting peptide may direct an AAV particle to the CNS (e.g., the cortex) after intravenous administration.

The targeting peptide may direct an AAV particle to the PNS (e.g., DRG) after intravenous administration.

A targeting peptide may vary in length. In certain embodiments, the targeting peptide is 3-20 amino acids in length. As non-limiting examples, the targeting peptide may be 3, 4, 5, 6, 7, 8, 9, 10, 11, 12, 13, 14, 15, 16, 17, 18, 19, 20 or 3-5, 3-8, 3-10, 3-12, 3-15, 3-18, 3-20, 5-10, 5-15, 5-20, 10-12, 10-15, 10-20, 12-20, or 15-20 amino acids in length.

Targeting peptides of the present disclosure may be identified and/or designed by any method known in the art. As a non-limiting example, the CREATE system as described in Deverman et al., (Nature Biotechnology 34(2):204-209 (2016)) and in International Patent Application Publication Nos. WO2015038958 and WO2017100671, the contents of each of which are herein incorporated by reference in their entirety, may be used as a means of identifying targeting peptides, in either mice or other research animals, such as, but not limited to, non-human primates (NHP).

Targeting peptides and associated AAV particles may be identified from libraries of AAV capsids comprised of targeting peptide variants. In certain embodiments, the targeting peptides may be 7 amino acid sequences (7-mers). In another embodiment, the targeting peptides may be 9 amino acid sequences (9-mers). The targeting peptides may also differ in their method of creation or design, with non-limiting examples including, random peptide selection, site saturation mutagenesis, and/or optimization of a particular region of the peptide (e.g., flanking regions or central core).

In certain embodiments, a targeting peptide library comprises targeting peptides of 7 amino acids (7-mer) in length randomly generated by PCR.

In certain embodiments, a targeting peptide library comprises targeting peptides with 3 mutated amino acids. In certain embodiments, these 3 mutated amino acids are consecutive amino acids. In another embodiment, these 3 mutated amino acids are not consecutive amino acids. In certain embodiments, the parent targeting peptide is a 7-mer. In another embodiment, the parent peptide is a 9-mer.

In certain embodiments, a targeting peptide library comprises 7-mer targeting peptides, wherein the amino acids of the targeting peptide and/or the flanking sequences are evolved through site saturation mutagenesis of 3 consecutive amino acids. In certain embodiments, NNK (N=any base; K=G or T) codons are used to generate the site saturated mutation sequences.

### CREATE System in Mice

In some embodiments, the AAV particles of the present disclosure are prepared via the CREATE system, as described in Deverman et al., (Nature Biotechnology 34(2):204-209 (2016)) and in International Patent Application Publication Nos. WO2015038958 and WO2017100671, the contents of each of which are herein incorporated by reference in their entirety. “CREATE” or “Cre-recombinant-based AAV targeted evolution” refers to an AAV capsid selection strategy that selects for capsids that transduce target tissues (e.g., CNS or PNS) following intravenous injection. The method has been demonstrated in a mouse model.

Libraries of AAV capsids with one or more targeting peptide inserts are developed and administered intravenously to transgenic mice. These transgenic cre-expressing mice may be developed for specific targeting, for example, GFAP-Cre mice may be used for targeting to astrocytes.

Variation of the targeting sequence as well as the transgenic animal model enables the selection of AAV variants with desired transduction profiles, for example, tropism to neurons or astrocytes, as compared to other AAV serotypes, including the parent AAV particle and capsid.

The CREATE method involves the generation of a library of targeting peptides which are then assembled into a viral genome backbone comprising a parent AAV capsid sequence. An AAV capsid library (AAV particles) is then generated, purified and administered to a transgenic animal (e.g., mouse). Target tissue is collected and AAV sequences selectively recovered from Cre expressing cells. These sequences are assessed and characterized for the identification of targeting peptides that lead to enrichment in a target tissue (i.e., enhanced transduction or tropism). Targeting peptides and associated AAV particles can then be generated for further testing and characterization. This process is considered one round of evolution or selection. In some embodiments, more than one round of evolution is conducted. As many as 15 rounds of selection may be conducted.

In more detail, the CREATE system uses a rAAV-Cap-in-cis-lox viral genome comprising AAV cap and regulator elements of the AAV rep genes and a Cre-invertible switch. Since this viral genome lacks a fully function rep gene necessary for AAV particle production, the rep is provided in trans. A modified AAV2/9 Rep-Cap plasmid may be provided, wherein stop-codons are provided in-frame to prevent the expression of VP1-VP3 proteins.

Capsid libraries are generated using the rAAV-Cap-in-cis-lox viral genome as a backbone. Targeting peptides are inserted into the parent AAV capsid protein (e.g., AAV9) at any position that results in the generation of a fully functional AAV capsid protein and AAV particle. As an exemplar, targeting peptides comprising 7 random amino acids were inserted between amino acids 588 and 589 of the VP1 sequence of K449 AAV9. Targeting peptides may be designed by any method known in the art. In certain embodiments, targeting peptides are generated using polymerase chain reaction (PCR).

AAV particles comprising capsid proteins with targeting peptide inserts are generated and viral genomes encoding a reporter (e.g., GFP) encapsulated within. These AAV particles (or AAV capsid library) are then administered to a transgenic mouse by intravenous delivery to the tail vein. Administration of these capsid libraries to cre-expressing mice results in expression of the reporter payload in the target tissue, due to the expression of Cre.

AAV particles and/or viral genomes may be recovered from the target tissue for identification of targeting peptides and associated AAV particles that are enriched, indicating enhanced transduction of target tissue. Standard methods in the art, such as, but not limited to next generation sequencing (NGS), viral genome quantification, biochemical assays, immunohistochemistry and/or imaging of target tissue samples may be used to determine enrichment. Total DNA may be extracted prior to the selective recovery of viral genomes using methods known in the art such as but not limited to Trizol based recovery extraction methods.

A target tissue may be any cell, tissue or organ of a subject. As non-limiting examples, samples may be collected from brain, spinal cord, dorsal root ganglia and associated roots, liver, heart, gastrocnemius muscle, soleus muscle, pancreas, kidney, spleen, lung, adrenal glands, stomach, sciatic nerve, saphenous nerve, thyroid gland, eyes (with or without optic nerve), pituitary gland, skeletal muscle (rectus femoris), colon, duodenum, ileum, jejunum, skin of the leg, superior cervical ganglia, urinary bladder, ovaries, uterus, prostate gland, testes, and/or any sites identified as having a lesion, or being of interest.

Targeting peptides and associated AAV capsid proteins and AAV particles identified using a CREATE system include AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4). AAVG2B5 (G2B5), and AAVPHP.S.

In certain embodiments, CREATE in mice is used to identify AAV capsids and/or targeting peptides having enhanced transduction of a target tissue (e.g., CNS or PNS).

### CREATE System in NHP

The CREATE system has proven efficacious in identifying targeting peptides for enhanced transduction to the CNS of mice after intravenous administration. However, translation of findings from mouse to human is not always straightforward. Modifying the CREATE system for non-transgenic animals or model systems that more closely resemble humans may help identify targeting peptides and associated AAV capsids and particles useful for the treatment of human disease.

For adaptation of the CREATE method to non-transgenic animals, another mechanism needs to be used to alter target tissues and/or cells to express Cre. In certain embodiments, AAV Cre-vectors may be used to transduce cells and induce subsequent Cre expression. In certain embodiments, these AAV Cre-vectors may be AAV1-Cre vectors.

These AAV Cre-vectors may comprise viral genomes with a cell-type specific promoter. These cell-type specific promoters may be, but are not limited to, CAG, UBC, EF1α, synapsin, GFAP, MBP, VGLUT, VGAT, Nav1.8 (SCN10A), parvalbumin, TH, ChaT, and/or any promoter known in the art.

In certain embodiments, these AAV-Cre vectors are delivered to a target tissue by intraparenchymal administration. In certain embodiments, the intraparenchymal administration is directly to the putamen of the subject. In certain embodiments, the intraparenchymal administration is directly to the thalamus of a subject. In certain embodiments, the intraparenchymal administration is directly to the cortex of a subject. In certain embodiments, the intraparenchymal administration is indirectly to the cortex of a subject. In certain embodiments, the intraparenchymal administration is simultaneously to one or more of the putamen, the thalamus and or the cortex of a subject, and may be bi-lateral administrations.

In certain embodiments, the subject is a non-human primate.

As for the CREATE method developed in mice, the AAV capsid libraries may be administered intravenously. In another embodiment, the AAV capsid libraries may be administered by intraparenchymal delivery. In certain embodiments, the AAV capsid library is administered prior to the delivery of the AAV-Cre vectors. In another embodiment, the AAV capsid library is administered after the delivery of the AAV-Cre vectors. The length of time between the administration of the AAV-Cre vectors and the AAV capsid libraries may be seconds, minutes, hours, days, weeks, or years.

The AAV capsid library may comprise AAV particles comprising a viral genome encoding a reporter (e.g., GFP). Only those cells of the target tissue (e.g., CNS or DRG) also expressing Cre (co-transduced by a Cre-vector administered intraparenchymally) will express the reporter.

Target tissues may be collected and analyzed for the identification of AAV particles and targeting peptides that lead to enrichment in the target tissue, i.e., enhanced transduction. Standard methods in the art may be used to assess, analyze, or characterize sample tissues and AAV sequences, including but not limited to, next generation sequencing, viral genome quantification, biochemical assays, immunohistochemistry and/or imaging.

In certain embodiments, CREATE in NHP is used to identify AAV capsids and/or targeting peptides having enhanced transduction of a target tissue (e.g., CNS or PNS).

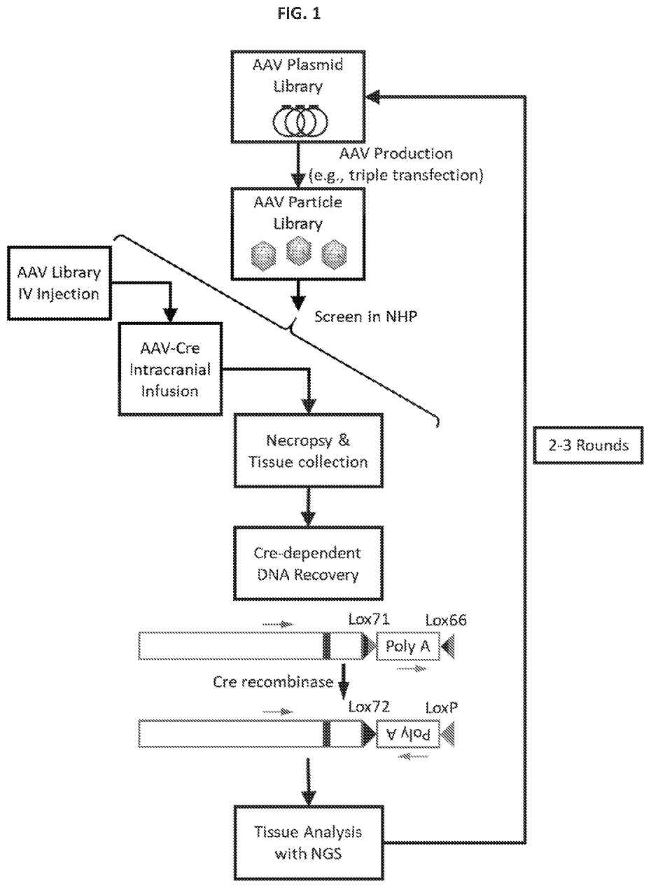

A non-limiting example of CREATE in NHP may be summarized as follows and is shown in FIG. 1. First, an AAV plasmid library is prepared and an AAV particle library is produced using triple transfection methods (other AAV library production methods may be used). The AAV particle library is administered to one or more NHP by intravenous administration at Day 0. Around Day 7, the NHP is administered AAV-Cre vectors by intracranial infusion. Around Day 20, necropsy is performed and tissues collected from brain, spinal cord, dorsal root ganglia, liver, heart, muscle, kidney, pancreas, spleen, lung, etc. Capsid DNA is captured by Cre-dependent DNA recovery from transduced tissues, using an rAAV-Cap-in-cis-lox rAAV genome wherein Cre inverts the polyadenylation sequence flanked by the lox71 and lox66 sites. PCR primers selectively amplify the Cre-recombined sequences. Recovered DNA can then be processed for and analyzed by next-generation sequencing (NGS) to identify AAV variants. Two to three rounds of this process may be conducted to identify enriched variants.

### Targeting Peptide Sequences

In certain embodiments the targeting peptide may comprise a sequence as set forth in SEQ ID NO: 4-14326, SEQ ID NO: 42973-42999, and SEQ ID NO: 43073-43341. These targeting peptides may be encoded by a sequence as set forth in SEQ ID NO: 14327-42972, SEQ ID NO: 43027-43053, and SEQ ID NO: 43342-43691. These sequences are shown in Tables 2, 3 and Table 4.

In certain embodiments, the targeting peptide may comprise an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the sequences shown in Table 2-4.

In certain embodiments, the targeting peptide may be encoded by a nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the sequences shown in Table 2-4.

In certain embodiments, a targeting peptide may comprise 4 or more contiguous amino acids of any of the targeting peptides disclosed herein. In certain embodiments the targeting peptide may comprise 4 contiguous amino acids of any of the sequences as set forth in SEQ ID NO: 4-14326, SEQ ID NO: 42973-42999, and SEQ ID NO: 43073-43341. In certain embodiments the targeting peptide may comprise 5 contiguous amino acids of any of the sequences as set forth in SEQ ID NO: 4-14326, SEQ ID NO: 42973-42999, and SEQ ID NO: 43073-43341. In certain embodiments the targeting peptide may comprise 6 contiguous amino acids of any of the sequences as set forth in SEQ ID NO: 4-14326, SEQ ID NO: 42973-42999, and SEQ ID NO: 43073-43341. In certain embodiments, the targeting peptide may comprise 7 contiguous amino acids of any of the sequences set forth in SEQ ID NO: 42973-42999, and SEQ ID NO: 43073-43341. In certain embodiments, the targeting peptide may comprise 8 contiguous amino acids of any of the sequences set forth in SEQ ID NO: 42973-42999, and SEQ ID NO: 43073-43341. In certain embodiments, the targeting peptide may comprise 9 contiguous amino acids of any of the sequences set forth in SEQ ID NO: 43073-43341. In certain embodiments, the targeting peptide may comprise 10 contiguous amino acids of any of the sequences set forth in SEQ ID NO: 43073-43341.

In certain embodiments, the AAV particle of the disclosure comprises an AAV capsid with a targeting peptide insert, wherein the targeting peptide has an amino acid sequence as set forth in any of SEQ ID NO: 4-14326. SEQ ID NO: 42973-42999, and SEQ ID NO: 43073-43341.

In certain embodiments, the AAV particle of the disclosure comprises an AAV capsid polynucleotide with a targeting nucleic acid insert, wherein the targeting nucleic acid insert has a nucleotide sequence as set forth in any of SEQ ID NO: 14327-42972, SEQ ID NO: 43027-43053, and SEQ ID NO: 43342-43691.

In certain embodiments, the AAV particle of the disclosure comprises an AAV capsid with a targeting peptide insert, wherein the targeting peptide has an amino acid sequence comprising at least 4 contiguous amino acids of any of the sequences as set forth in any of SEQ ID NO: 4-14326, SEQ ID NO: 42973-42999, and SEQ ID NO: 43073-43341.

In certain embodiments, the AAV particle of the disclosure comprises an AAV capsid with a targeting peptide insert, wherein the targeting peptide has an amino acid sequence substantially comprising any of the sequences as set forth in any of SEQ ID NO: 4-14326, SEQ ID NO: 42973-42999, and SEQ ID NO: 43073-43341.

In certain embodiments, the AAV particle of the disclosure comprises an AAV capsid polynucleotide with a targeting nucleic acid insert, wherein the targeting nucleic acid insert has a nucleotide sequence substantially comprising any of those set forth as SEQ ID NO: 14327-42972, SEQ ID NO: 43027-43053, and SEQ ID NO: 43342-43691.

The AAV particle of the disclosure comprising a targeting nucleic acid insert, may have a polynucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, identity to the parent capsid sequence.

The AAV particle of the disclosure comprising a targeting peptide insert, may have an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, identity to the parent capsid sequence.

In one aspect, the disclosure provides peptides (targeting peptides) with amino acid sequences set forth as any of SEQ ID NO: 4-14326, SEQ ID NO: 42973-42999 or SEQ ID NO: 43073-43341. Nucleic acid sequences encoding these peptides are also provided and are set forth as any of SEQ ID NO: 14327-42972, SEQ ID NO: 4300-43053 or SEQ ID NO: 43342-43691.

Lengthy table referenced here
US20220281922A1-20220908-T00001
Please refer to the end of the specification for access instructions.

Lengthy table referenced here
US20220281922A1-20220908-T00002
Please refer to the end of the specification for access instructions.

The targeting peptide may direct an AAV particle to a cell or tissue of the nervous system such as but not limited to the dorsal root ganglia (DRG) and/or the trigeminal ganglia (TRG). Such targeting peptides may include but are not limited to the peptides listed in Table 4.

TABLE 4
DRG and TRG targeting peptides

| SEQ | Peptide | SEQ ID | |
|---|---|---|---|
| ID NO | Sequence | NO: | Nucleotide Sequence(s) |
| 43073 | PSSAVRTSLAQ | 43342 | CCCTCATCAGCCGTCCGAACGTCACTTGCCCAA |
| | | 43343 | CTTTCGTCTGCGGTTAGGACGTCTTTGGCACAG |
| 43074 | EPAAVRTSLAQ | — | |
| 43075 | DPAAVRTSLAQ | — | — |
| 43076 | AQQAPNHSLAQ | — | — |
| 43077 | AQQASSSSLAQ | — | — |
| 43078 | AQQASTKSLAQ | — | — |
| 43079 | AQQAGPTSLAQ | — | — |
| 43080 | AQQAVRESNAQ | — | — |
| 43081 | AQQAVRNSTAQ | — | — |
| 43082 | AQQAVRTSAGV | — | — |
| 43083 | AQAGQIHHNAQ | 43344 | GCTCAGGCTGGTCAGATTCATCATAATGCTCAG |
| | | 43345 | GCCCAAGCTGGTCAGATTCATCATAATGCACAG |
| 43084 | AQAGSDKAKAQ | 43346 | GCCCAAGCCGGCTCAGACAAGGCCAAGGCCCAA |
| | | 43347 | GCTCAGGCTGGTAGTGATAAGGCTAAGGCTCAG |
| | | 43348 | GCCCAAGCGGGTAGTGATAAGGCTAAGGCACAG |
| 43085 | AQALNGARLAQ | 43349 | GCCCAAQQCCTTAACGGCGCCCGACTTGCCCAA |
| | | 43350 | GCGCAAGCCCTTAACGGCGCCCGACTTGCCCAA |
| | | 43351 | GCGCAGGCGTTGAATGGGGCGAGGTTGGCGCAG |
| 43086 | AQASQRALPAQ | 43352 | GCCCAAGCTTCGGATCGTGCGCTGCCTGCACAG |
| | | 43353 | GCGCAGGCGTCGGATAGGGCGTTGCCGGCGCAG |
| 43087 | AQASVLAHPAQ | 43354 | GCCCAAGCGTCTGTCCTTGCTCATCCTGCACAG |
| | | 43355 | GCTCAGGCTAGTGTTCTTGCTCATCCTGCTCAG |
| 43088 | AQAVKGLDLAQ | 43356 | GCACAAGCAGTAAAGGGATTAGACTTAGCACAA |
| | | 43357 | GCTCAGCCTGTTAAGGGTCTTGATCTIGCTCAG |
| | | 43358 | GCGCAGGCGGTGAAGGGGTTGGATTTGGCGCAG |
| | | 43359 | GCCCAAGCCGTCAAGGGCCTTGACCTTGCCCAA |
| | | 43360 | GCCCAAGCGGTGAAGQGGTTGGATTTGGCACAG |
| 43089 | AQDATRALAAQ | 43361 | GCGCAGGATGCGACGAGGGCGTTGGCGGCGCAG |
| | | 43362 | GCACAAGACGCAACTAGGGCATTAGCAGCACAA |
| 43090 | AQDTQVARSAQ | 43363 | GCTCAGGATACTCAGGTTGCTCGTAGTGCTCAG |
| | | 43364 | GCGCAGGATACGCAGGTGGCGAGGTCGGCGCAG |
| 43091 | AQDTRGIMAAQ | 43365 | GCCCAAGACACGCGAGGCATCATGGCCGCCCAA |
| | | 43366 | GCTCAGGATACTCGTGGTATTATGGCTGCTCAG |
| 43092 | AQEMRGLAIAQ | 43367 | GCACAAGAAATGAGGGGATTAGCAATAGCACAA |
| | | 43368 | GCTCAGGAGATGCGTGGTCTTGCTATTGCTCAG |
| 43093 | AQFAGSNAAAQ | 43369 | GCGCACTTTGCGGGGTCGAATGCGGCGGCGCAG |
| | | 43370 | GCACAATTCGCAGGAAGCAACGCAGCAGCACAA |
| | | 43371 | GCCCAATTTGCGGGGTCTAATGCTGCTGCACAG |
| 43094 | AQGADLRNPAQ | 43372 | GCGCAGGGTGCTGATCTTCGTAATCCTGCTCAG |
| | | 43373 | GCGCAGGGGGCGGATTTGAGGAATCCGGCGCAG |
| 43095 | AQGFSNHDPAQ | 43374 | GCCCAAGGCTTTTCAAACCATGACCCCGCCCAA |
| | | 43375 | GCCCAAGGGTTTTCGAATCATGATCCTGCACAG |
| 43096 | AQGLTATVPAQ | 43376 | GCCCAAGGTCTGACTGCTACTGTTCCGGCACAG |
| | | 43377 | GCTCAGGGTCTTACTGCTACTGTTCCTGCTCAG |
| 43097 | AQGPTSDRQAQ | 43378 | GCCCAAGGTCCGACGTCGGATCGGCAGGCACAG |
| | | 43379 | GCCCAAGGGCCGACGTCGGATCGGCAGGCACAG |
| 43098 | AQGRMDSRPAQ | 43380 | GCGCAGCGGAGGATGGATTCGAGGCCGGCGCAG |
| | | 47381 | GCCCAAGGTCCTATGGATAGTCGGCCTGCACAG |
| 43099 | AQGSSPQATAQ | 43382 | GCTCAGGGTAGTAGTCCTCAGGCTACTGCTCAG |
| | | 43383 | GCGCAGGGGTCGTCGCCGCAGGCGACGGCGCAG |
| 43100 | AQHAKITYDAQ | 43384 | GCCCAACATGCCAAGATCACGTATGACGCCCAA |
| | | 43385 | GCTCAGCATGCTAAGATTACTTATGATGCTCAG |
| 43101 | AQIADSRNSAQ | 43386 | GCGCAGATTGCGGATTCGAGGAATTCGGCGCAG |
| | | 43387 | GCCCAAATCGCCGACTCACGAAACTCAGCCCAA |
| 43102 | AQINVANRSAQ | 43388 | GCCCAAATCAACGTCGCCAACCGATCAGCCCAA |
| | | 43389 | GCCCAAATTAATGTTGCTAATAGGTCTGCACAG |
| | | 43390 | GCGCAGATTAATGTGGCGAATAGGTCGGCGCAG |
| 43103 | AQIRSTNNSAQ | 43391 | GCTCAGATTCGTAGTACTAATAATAGTGCTCAG |
| | | 43392 | GCACAAATAAGGAGCACTAACAACAGCGCACAA |
| 43104 | AQKNGIVDSAQ | 43393 | GCTCAGAAGAATGGGATTGTGGATTCGGCGCAG |
| | | 43394 | GCACAAAAGAACGGAATAGTAGACAGCGCACAA |
| | | 43395 | GCCCAAAAGAATGGTATTGTGGATTCGGCACAG |
| 43105 | AQKSGTPALAQ | 43396 | GCGCAGAAGTCGGGGACTCCGGCGTTGGCGCAG |
| | | 43397 | GCCCAAAAGTCAGGCACGCCCGCCCTTGCCCAA |
| 43106 | AQKSTQSPDAQ | 43398 | GCCCAAAAGTCGACGCAGAGTCCTGATGCACAG |
| | | 43399 | GCTCAGAAGAGTACTCAGAGTCCTGATGCTCAG |
| 43107 | AQKVATHPVAQ | 43400 | GCACAAAAGGTAGCAACTCACCCAGTAGCACAA |
| | | 43401 | GCCCAAAAGGTGGCGACGCATCCTGTGGCACAG |
| 43108 | AQLAGVSHAAQ | 43402 | GCCCAACTTGCCGGCGTCTCACATGCCGCCCAA |
| | | 43403 | GCACAATTAGCAGGAGTAAGCCACGCAGCACAA |
| | | 43404 | GCGCAGTTGGCGGGGGTGTCGCATGCGGCGCAG |
| 43109 | AQLKNSDSYAQ | 43405 | GCGCAGTTGAAGAATTCGGATTCGTATGCGCAG |
| | | 43406 | GCCCAACTTAAGAACTCAGACTCATATGCCCAA |
| 43110 | AQLLGGSDRAQ | 43407 | GCACAATTATTAGGAGGAAGCGACAGGGCACAA |
| | | 43408 | GCGCAGTTGTTGGGGGGGTCGGATAGGGCGCAG |
| 43111 | AQLMGSGEGAQ | 43409 | GCCCAACTTATGGGGTCTGGGGAGGGGGCACAG |
| | | 43410 | GCACAATTAATGGGAAGCGGAGAAGGAGCACAA |
| 43112 | AQLQGAALPAQ | 43411 | GCCCAATTACAAGGAGCAGCATTACCACCACAA |
| | | 43412 | GCGCAATTACAAGGAGCAGCATTACCAGCACAA |
| | | 43413 | GCACAATTACAAGGAGCAGCATTACCAGCACAA |
| 43113 | AQMQVNGNGAQ | 43414 | GCGCAGATGCAGGTGAATGGGAATGGGGCGCAG |
| | | 43415 | GCTCAGATGCAGGTTAATGGTAATGGTGCTCAG |
| 43114 | AQMSMSVGKAQ | 43416 | GCGCAGATGTCGATGTCGGTGGGGAAGGCGCAG |
| | | 43417 | GCCCAAATGAGTATGTCTGTGGGTAAGGCACAG |
| 43115 | AQNAVNGRPAQ | 43418 | GCCCAAAATGCGGTTAATGGTCGTCCTGCACAG |
| | | 43419 | GCGCAGAATGCGGTGAATGGGAGGCCGGCGCAG |
| 43116 | AQNEGHSAKAQ | 43420 | GCACAAAACGAAGGACACAGCGCAAAGGCACAA |
| | | 43421 | GCCCAAAACGAAGGCCATTCAGCCAAGGCCCAA |
| 43117 | AQNGFNRSAAQ | 43422 | GCGCAGAATGGGTTTAATAGGTCGGCGGCGCAG |
| | | 43423 | GCCCAAAATGGGTTTAATCGTAGTGCGGCACAG |
| 43118 | AQNNGVHLRAQ | 43424 | GCACAAAACAACGGAGTACACTTAAGGGCACAA |
| | | 43425 | GCCCAAAACAACGGCGTCCATCTTCGAGCCCAA |
| 43119 | AQNPKSIPGAQ | 43426 | GCACAAAACCCAAAGAGCATACCAGGAGCACAA |
| | | 43427 | GCTCAGAATCCTAAGAGTATTCCTGGTGCTCAG |
| 43120 | AQNTLSHAKAQ | 43428 | GCACAAAACACTTTAAGCCACGCAAAGGCACAA |
| | | 43429 | GCCCAAAACACGCTTTCACATGCCAAGGCCCAA |
| 43121 | ACPAVSGGLAQ | 43430 | GCTCAGCCTGCTGTTAGTGGTGGTCTTGCTCAG |
| | | 43431 | GCGCAGCCGGCGGTGTCGGGGGGGTTGGCGCAG |
| 43122 | AQPEHAVRTAQ | 43432 | GCACAACCAGAACACGCAGTAAGGACTGCACAA |
| | | 43433 | GCCCAACCGGAGCATGCGGTTCGTACTGCACAG |
| 43123 | AQPGLDTTRAQ | 43434 | GCGCAGCCTGGGTTGGATACGACGAGGGCGCAG |
| | | 43435 | GCCCAACCCGGCCTTGACACGACGCGAGCCCAA |
| | | 43436 | GCGCAGCCGGGGTTGGATACGACGAGGGCGCAG |
| 43124 | AQPISTAAYAQ | 43437 | GCGCAGCCGATTTCGACGGCGGCGTATGCGCAG |
| | | 43438 | GCCCAACCCATCTCAACGGCCGCCTATGCCCAA |
| 43125 | AQPRPESPSAQ | 43439 | GCGCAACCAAGGCCAGAAAGCCCAAGCGCACAA |
| | | 13440 | GCTCAGCCTCGTCCTGAGAGTCCTAGTGCTCAG |
| 43126 | AQPTRSVTEAQ | 43441 | GCGCAGCCGACGAGGTCGGTGACGGAGGCGCAC |
| | | 43442 | GCTCAGCCTACTCGTAGTGTTACTGAGGCTCAG |
| 43127 | AQQHKTAQVAQ | 43443 | GCCCAACAACATAAGACGGCCCAAGTCGCCCAA |
| | | 43444 | GCGCAGCAGCATAAGACGGCGCAGGTGGCGCAG |
| 43128 | AQQTGVLAAAQ | 43445 | GCGCAGCAGACGGGGGTGTTGGCGGCGGCACAG |
| | | 43446 | GCACAACAAACTGGAGTATTAGCAGCAGCACAA |
| 43129 | AQQTVSMMSAQ | 43447 | GCTCAGCAGACTGTTAGTATGATGAGTGCTCAG |
| | | 43448 | GCGCAGCAGACGGTGTCGATGATGTCGGCGCAG |
| 43130 | AQSASSNQPAQ | 43449 | GCGCAGTCGGCGTCGTCGAATCAGCCGGCGCAG |
| | | 43450 | GCACAAAGCGCAAGCAGCAACCAACCAGCACAA |
| 43131 | AQSGGGPVRAQ | 43451 | GCTCAGAGTGGTGGTGGTCCTGTTCGTGCTCAG |
| | | 43452 | GCACAAAGCGGAGGAGGACCAGTAAGGGCACAA |
| 43132 | AQSGLQPKMAQ | 43453 | GCGCAGTCGGGGTTGCAGCCGAAGATGGCGCAG |
| | | 43454 | GCCCAATCAGGCCTTCAACCCAAGATGGCCCAA |
| 43133 | AQSGTLQRAAQ | 43455 | GCACAAAGCGGAACTTTACAAAGGGCAGCACAA |
| | | 43456 | GCCCAATCTGGGACGTTGCAGAGGGCTGCACAG |
| 43134 | AQSTNVSSAAQ | 43457 | GCACAAAGCATAAACGTAAGCAGCGCAGCACAA |
| | | 43458 | GCGCAGTCGATTAATGTGTCGTCGGCGGCGCAG |
| 43135 | AQSKSANLPAQ | 43459 | GCACAAAGCAAGAGCGCAAACTTACCAGCACAA |
| | | 43460 | GCCCAATCAAAGTCACCCAACCTTCCCGCCCAA |
| 43136 | AQSSGDSTKAQ | 43461 | GCGCAGTCGTCGGGGGATTCGACGAAGGCGCAG |
| | | 43462 | GCTCAGAGTAGTGGTGATAGTACTAAGGCTCAG |
| | | 43463 | GCCCAATCATCAGGCGACTCAACGAAGGCCCAA |
| | | 43464 | GCACAAAGCAGCGGAGACAGCACTAAGGCACAA |
| 13137 | AQSSGQGQVAQ | 43465 | GCCCAATCATCAGGCCAAGGCCAAGTCGCCCAA |
| | | 13466 | GCACAAAGCAGCGGACAAGGACAAGTAGCACAA |
| 43138 | AQSTTLQNVAQ | 43467 | GCCCAATCAACGACGCTTCAAAACGTCGCCCAA |
| | | 43468 | GCACAAAGCACTACTTTACAAAACGTAGCACAA |
| 43139 | AQTETPVSRAQ | 43469 | GCCCAAACGGAAACTCCCGTCTCACGAGCCCAA |
| | | 43470 | GCCCAAACGGAAACGCCCGTCTCACGAGCCCAA |
| 43110 | AQTGAASPIAQ | 43471 | GCCCAAACGGGCGCCGCCTCACGACTTGCCCAA |
| | | 43472 | GCACAAACTGGAGCAGCAAGCAGGTTAGCACAA |
| 43141 | AQTINPAVRAQ | 43473 | GCACAGACGATTAATCCGGCGGTGAGGGCGCAG |
| | | 43474 | GCTCAGACTATTAATCCTGCTGTTCGTGCTCAG |
| | | 43475 | GCGCAGACGATTAATCCGGCGGTGAGGGCGCAG |
| 43142 | AQTPSSASSAQ | 43476 | GCGCAGACGCCGTCGTCGGCGTCGTCGGCGCAG |
| | | 43477 | GCACAAACTCCAAGCAGCGCAAGCAGCGCACAA |
| 43143 | AQTSHTMSTAQ | 43478 | GCCCAAACGTCTCATACTATGAGTTTTGCACAG |
| | | 43479 | GCTCAGACTAGTCATACTATGAGTTTTGCTCAG |
| 43144 | AQTSTQSVNAQ | 43480 | GCCCAAACGTCGACTCAGAGTGTGAATGCACAG |
| | | 43481 | GCGCAGACGTCGACGCAGTCGGTGAATGCGCAG |
| 43145 | AQVHAGAEVAQ | 43482 | GCGCAGGTGCATGCGGGGGCGGAGGTTGCGCAG |
| | | 43483 | GCGCAGGTTCATGCGGGGGCGGAGGTGGCGCAG |
| | | 43484 | GCACAGGTGCATGCGGGGGCGGAGTTGGCGCAG |
| | | 43485 | GCGCAGGTGCATGCGGGGGCGGAGGTGGCGCAG |
| 43146 | AQVKDGLTSAQ | 43486 | GCGCAGGTGAAGGATGGGTTGACGTCGGCGCAG |
| | | 43487 | GCACAAGTAAAGGACGGATTAACTAGCGCACAA |
| | | 43488 | GCTCAGGTTAAGGATGGTCTTACTAGTGCTCAG |
| | | 43489 | GCGCAGGTTAAGGATGGTCTTACTAGTGCTCAG |
| 43147 | AQVNNQSAKAQ | 43490 | GCCCAAGTGAATAATCAGTCGGCTAAGGCACAG |
| | | 43491 | GCCCAAGTAAACAACCAAAGCGCAAAGGCACAA |
| | | 43492 | GCACAAGTAAACAACCAAAGCGCAAAGGCACAA |
| | | 43493 | GCGCAGGTGAATAATCAGTCGGCGAAGGCGCAG |
| 43148 | AQVRDGIPPAQ | 43494 | GCTCAGGTTCGTGATGGTATTCCTCCTGCTCAG |
| | | 43495 | GCACAAGTAAGGGACGGAATACAACCAGCACAA |
| 43149 | AQVSPGMAGAQ | 43496 | GCTCAAGTAAGCCCAGGAATGGCAGGAGCACAA |
| | | 43497 | GCCCAAGTTAGTCCTGGGATGGCTGGTGCACAG |
| 43150 | AQVVNELRGAQ | 43498 | GCACAAGTAGTAAACGAATTAAGGGGAGCACAA |
| | | 43499 | GCCCAAGTGGTTAATGAGTTGCGGGGTGCACAG |
| 43151 | AQVVPHNGLAQ | 43500 | GCCCAAGTCGTCCCCCATAACGGCCTTGCCCAA |
| | | 43501 | GCACAAGTAGTACCACACAACGGATTAGCACAA |
| 43152 | AQHAKIMYDAQ | 43502 | GCCCAACATGCCAAGATCATGTATGACGCCCAA |
| 43153 | AQAVSSQVSAQ | 43503 | GCGCAGGCGGTGTCGTCGCAGGTGTCGGCGCAG |
| 43154 | AQGLSVVNMAQ | 43504 | GCCCAAGGTCTTAGTGTTGTTAATATGGCACAG |
| 43155 | AQRSMPTLSAQ | 43505 | GCCCAACGATCAATGCCCACGCTTTCAGCCCAA |
| 43156 | AQPVNTQPVAQ | 43506 | GCTCAGCCTGTTAATACTCAGCGTGTTGCTCAG |
| 43157 | AQVSGDGSRAQ | 43507 | GCGCAGGTGTCGGGGGATGGGTCGAGGGCGCAG |
| 43158 | AQDLNNRPYAQ | 43508 | GCCCAAGATCTGAATAATCGGCCTTATGCACAG |
| 43159 | AQLSESSHGAQ | 43509 | GCCCAATTGTCTGAGTCGAGTCATGGTGCACAG |
| 43169 | AQVSNNSTLAQ | 43510 | GCACAACTAAGCAACAACAGCACTTTAGCACAA |
| 43160 | AQLQGAALQAQ | 43511 | GCACAATTACAAGGAGCAGCATTACAAGCACAA |
| 43162 | AQLRMPSNVAQ | 43512 | GCCCAACTTCGAATGCCCTCAAACGTCGCCCAA |
| 43163 | AQMPTSQPVAQ | 43513 | GCGCAGATGCCGACGTCGCAGCCGGTGGCGCAG |
| 43164 | AQYETQLSHAQ | 43514 | GCGCAGTATGAGACGCAGTTGTCGCATGCGCAG |
| 43165 | AQTRIQPLPAQ | 43515 | GCGCAGACGAGGATTCAGCCGTTGCCGGCGCAG |
| 43166 | AQPAPSGNTAQ | 43516 | GCGCAGCCGGCGCCGTCGGGGAATACGGCGCAG |
| 43167 | AQIHRMYSAAQ | 43517 | GCCCAAGAGCTTCGGATGTATAGTGCTGCACAG |
| 43168 | AQKESVPISAQ | 43518 | GCCCAAAAGGAATCAGTCCCCATCTCAGCCCAA |
| 43169 | AQVREVTLVAQ | 43519 | GCGCAGGTGAGGGAGGTGACGTTGGTGGCGCAG |
| 43170 | AQISSQLALAQ | 43520 | GCCCAAATCTCATCACAACTTGCCCTTGCCCAA |
| 43171 | AQSLGLTQHAQ | 43521 | GCTCAGAGTCTTGGTCTTACTCAGCATGCTCAG |
| 43172 | AQDLRMYSAAQ | 43522 | GCCCAAGATCTTCGGATGTATAGTGCTGCACAG |
| 43173 | AQTVAASRLAQ | 43523 | GCACAAACTGTAGCAGCAAGCAGGTTAGCACAA |
| 43174 | AQEVSVNVSAQ | 43524 | GCCCAAGAAGTCTCAGTCAACGTCTCCGCCCAA |
| 43175 | AQLNALATTAQ | 43525 | GCGCAGTTGAATGCGTTGGCGACGACGGCGCAG |
| 43176 | AQGTSLKGPAQ | 43526 | GCGCAAGGAACTAGCTTAAAGGGACCAGCACAA |
| 43177 | AQFPSTGVRAQ | 43527 | GCCCAATTTCCATCAACGGGCGTCCGAGCCCAA |
| 43178 | AQDHASESLAQ | 43528 | GCGCAGGATCATGCGTCGGAGTCGTTGGCGCAG |
| 43179 | AQSELSRHLAQ | 43529 | GCACAAAGCGAATTAAGCAGGCACTTAGCACAA |
| 43180 | AQNVFNRSAAQ | 43530 | GCGCAGAATGTGTTTAATAGGTCGGCGGCGCAG |
| 43181 | AQAQIATRMAQ | 43531 | GCGCAGGCGCAGATTGCGACGAGGATGGCGCAG |
| 43182 | AQTQMVTPAAQ | 43532 | GCACAAACTCAAATGGTAACTCCAGCAGCACAA |
| 43183 | AQRSSDSNCAQ | 43533 | GCCCAACGATCATCAGACTCAAACTGCGCCCAA |
| 43184 | AQTYGSEGSAQ | 43534 | GCCCAAACTTATGGTAGTGAGGGGTCGGCACAG |
| 43185 | AQINVANRSSQ | 43535 | GCCCAAATCAACGTCGCCAACCGATCATCCCAA |
| 43186 | AQLPTSPLDAQ | 43536 | GCGCAGTTGCCCACGTCGCCGTTGGATGCGCAG |
| 43187 | AQAKGGINGAQ | 43537 | GCCCAAGCGAAGGGTGGTATTAATGGGGCACAG |
| 43188 | AQQTVSMMLAQ | 43538 | GCTCAGCAGACTGTTAGTATGATGATTGCTCAG |
| 43189 | AQVHVGAEVAQ | 43539 | GCCCACGTGCATGTGGCGGCGGAGGTGGCGCAG |
| 43190 | AQESNLAALAQ | 43540 | GCTCAGGAGAGTAATCTTGCTGCTCTTGCTCAG |
| 43191 | AHVHAGAEVAQ | 43541 | GCGCATGTGCATGCGGGGGCGGAGGTGGCGCAG |
| 43192 | AQPALKTDPAQ | 43542 | GCACAACCCGCCCTTAAGACGGACCCCGCCCAA |
| 43193 | AQSLTTVGRAQ | 43543 | GCCCAATCTCTGACTACGGTGGGTCGGGCACAG |
| 43194 | AQDSRSNITAQ | 43544 | GCCCAAGATAGTAGGTCGAATATTACTGCACAG |
| 43195 | AQPRGQDGYAQ | 43545 | GCCCAACCCCGAGGCCAAGACGGCTATGCCCAA |
| 43196 | AQELNTSKDAQ | 43546 | GCGCAGGAGTTGAATACGTCGAAGGATGCGCAG |
| 43197 | AQYLGASQLAQ | 43547 | GCCCAATATCTTGGCGCCTCACAACTTGCCCAA |
| 43198 | AQYESRMPNAQ | 43548 | GCCCAATATGAGAGTCGTATGCCTAATGCACAG |
| 43199 | AQRGTQNHLAQ | 43549 | GCACAAAGGGGAACTCAAAACCACTTAGCACAA |
| 43200 | AQLTASH1LAQ | 43550 | GCCCAATTGACTGCGTCGCATATTCTGGCACAG |
| 43201 | AQYLSDKRTAQ | 43551 | GCGCAGTATTTGTCGGATAAGAGGACGGCGCAG |
| 43202 | AKELNTSKDAQ | 43552 | GCCAAAGAACTTAACACGTCAAAGGACGCCCAA |
| 43203 | AQFSTPSTTAQ | 43553 | GCACAATTCAGCACTCCAAGCACTACTGCACAA |
| 43204 | AQNYNSHMGAQ | 43554 | GCCCAAAATGTTAATTCTCATATGGGGGCACAG |
| 43205 | AQMPVST1SAQ | 43555 | GCGCAGATGCCGGTGTCGACGATTTCGGCGCAG |
| 43206 | AQSIGQGQVAQ | 43556 | GCACAAAGCATCGGACAAGGACAAGTAGCACAA |
| 43207 | AQVLREQSLAQ | 43557 | GCGCAGGTGTTGAGGGAGCAGTCGTTGGCGCAG |
| 43208 | AQRLAANNVAQ | 43558 | GCACAAAGGTTAGCAGCAAACAACGTAGCACAA |
| 43209 | AQNRVSESVAQ | 43559 | GCACAAAACAGGGTAAGCGAAAGCGTAGCACAA |
| 43210 | AQQHNGAQLAQ | 43560 | GCACAACAACACAACGGAGCACAATTAGCACAA |
| 43211 | AQASRSLSTAQ | 43561 | GCTCAGGCTAGTCGTAGTCTTAGTACTGCTCAG |
| 43212 | AQPTSGNLTAQ | 43562 | GCGCAGTTTACGTCGGGGAATTTGACGGCGCAG |
| 43213 | AQTVSNR7LAQ | 43563 | GCACAAACTGTAAGCAACAGGACTTTAGCACAA |
| 43214 | AQYDHSGTKAQ | 43564 | GCACAATATGACCACAGCGGAACTAAGGCACAA |
| 43215 | AQNVTSVTAAQ | 41565 | GCACAAAACGTAACTAGCGTAACTGCAGCACAA |
| 43216 | AQSQLAGAAAQ | 43566 | GCCCAATCGCAGCTTGCTGGTGCGGCGGCACAG |
| 43217 | AQSLRTPMLAQ | 43567 | GCTCAGAGTCTTCGTACTCCTATGCTTGCTCAG |
| 43218 | AQTMRSSVTAQ | 41568 | GCCCAAACGATGCGATCATCAGTCACGGCCCAA |
| 43219 | AQMISSSLAAQ | 43569 | GCCCAAATGATCTCATCATCACTTGCCGCCCAA |
| 43220 | AQSRMPLPEAQ | 43570 | GCCCAATCACGAATGCCCCTTCCCGAAGCCCAA |
| 43221 | AQARQSSALAQ | 41571 | GCCCAAGCCCGACAATCATCAGCCCTTGCCCAA |
| 43222 | AQAITGSTYAQ | 43572 | GCTCAGGCTATTACTGGTAGTACTTATGCTCAG |
| 43223 | AQPVLDTTRAQ | 41573 | GCGCAGCCGGTGTTGGATACGACGAGGGCGCAG |
| 43224 | AQPNGLSQPAQ | 43574 | GCACAACCAAACGGATTAAGCCAACCAGCACAA |
| 43225 | AQYSALMLSAQ | 43575 | GCGCAGTATTCGGCGTTGAATTTGTCGGCGCAG |
| 43226 | AQEHASRNPAQ | 41576 | GCGCAGGAGCATGCGTCGAGGAATCCGGCGCAG |
| 43227 | AQGITHQTVAQ | 43577 | GCCCAAGGGATTACGCATCAGACGGTGGCACAG |
| 43228 | AQAYGLPQDAQ | 41578 | GCACAAGCATATGGATTACCACAAGACGCACAA |
| 43229 | AQSTPTPSLAQ | 43579 | GCACAAAGCACTCCAACTCCAAGCTTAGCACAA |
| 43230 | AQSSHLVSSAQ | 43580 | GCCCAATCATCACATCTTGTCTCATCAGCCCAA |
| 43231 | AQIFQGMPNAQ | 41581 | GCGCAGATTTTTCAGGGGATGCCGAATGCGCAG |
| 43232 | AQMQTARTLAQ | 43582 | GCTCAGATGCAGACTGCTCGTACTCTTGCTCAG |
| 43233 | AQYTPRNAPAQ | 43583 | GCACAATATACTCCAAGGAACGCACCAGCACAA |
| 43234 | AQGFSTGTNAQ | 43584 | GCCCAAGGCTTTTCAACGGGCACGAACGCCCAA |
| 43215 | AQLQYTPSRAQ | 43585 | GCTCAGCTTCAGTATACTCCTAGTCGTGCTCAG |
| 43236 | AQAVRSTSAAQ | 43586 | GCGCAGGCGGTGAGGTCGACGTCGGCGGCGCAG |
| 43237 | AQTEVSIRQAQ | 43587 | GCTCAGACTGAGGTTAGTATTCGTCAGGCTCAG |
| 43218 | AQYGVQSATAQ | 43588 | GCCCAATATGGTGTTCAGTCGGCGACTGCACAG |
| 43239 | AQASTGSTYAQ | 43589 | GCTCAGGCTACTACTGGTAGTACTTATGCTCAG |
| 43240 | AQNTSSKDPAQ | 43590 | GCCCAAAATACTAGTAGTAAGGATCCGGCACAG |
| 43241 | AQAGSQGALAQ | 43591 | GCTCAGGCTGGTAGTCAGGGTGCTCTTGCTCAG |
| 45242 | AQLTRSELSAQ | 43592 | GCGCAGTTGACGAGGTCGGAGTTGTCGGCGCAG |
| 43243 | AQFVSGSNAAQ | 43593 | GCCCAATTTGTGAGTGGTTCGAATGCGGCACAG |
| 43244 | AQLITSYPSAQ | 41594 | GCCCAACTTACGACGTCATATCCCTCAGCCCAA |
| 43245 | AQSSVAiQLAQ | 43595 | GCGCAGTCGTCGGTGGCGATTCAGTTGGCGCAG |
| 43246 | AQFSTSLSLAQ | 43596 | GCTCAGTTTAGTACTAGTCTTAGTCTTGCACAG |
| 43247 | AQKNLHD7TAQ | 41597 | GCACAAAAGAACTTACACGACACTACTGCACAA |
| 43248 | AQASISAG1AQ | 43598 | GCTCAGGCTAGTATTAGTGCTGGTATTGCTCAG |
| 43249 | AQRVSNHDGAQ | 43599 | GCCCAACGAGTCTCAAACCATGACGGCGCCCAA |
| 43250 | AQESNLAARAQ | 43600 | GCTCAGGAGAGTAATCTTGCTGCTCGTGCTCAG |
| 43251 | AQLNAPQ5SAQ | 43601 | GCGCAGTTGAATGCGCCGCAGTCGTCGGCGCAG |
| 43252 | AQSVGMHGDAQ | 43602 | GCTCAGAGTGTTGGTATGCATGGTGATGCTCAG |
| 43253 | AQMTTRPTPAQ | 43603 | GCTCAGAATACTACTCGTCCTACTCCTCCTCAG |
| 43254 | AQIRQAQGAAQ | 43604 | GCCCAAATCCGACAAGCCCAAGGCGCCGCCCAA |
| 43255 | AQPTNFGTAAQ | 43605 | GCCCAACCTACGAATTTTGGTACGGCTGCACAG |
| 43256 | AQADFPRQSAQ | 43606 | GCGCAGGCGGATTTTCCGAGGCAGTCGGCGCAG |
| 43257 | AQNGNSHMGAQ | 43607 | GCCCAAAATGGTAATTCTCATATGGGGGCACAG |
| 43258 | AQPREQSSPAQ | 43608 | GCGCAGCCGAGGGAGCAGTCGTCGCCGGCGCAG |
| 43259 | AQEASGNARAQ | 43609 | GCCCAAGAGGCGTCTGGGAATGCTCGGGCACAG |
| 43260 | AQEAQSKRPAQ | 43610 | GCACAAGAAGCACAAAGCAAGAGGCCAGCACAA |
| 43261 | AQSSSNNVRAQ | 43611 | GCTCAGAGTAGTAGTAATAATGTTCGTGCTCAG |
| 43262 | AQSPSKGAVAQ | 43612 | GCACAAAGCCCAAGCAAGGGAGCAGTAGCACAA |
| 43263 | AQMLTTSKCAQ | 43613 | GCCCAAATGCTTACGACGTCAAAGGGCGCCCAA |
| 43264 | AQLEPSSPRAQ | 43614 | GCTCAGCTTGAGCCTAGTAGTCCTCGTGCTCAG |
| 43265 | AQMTGPITNAQ | 43615 | GCCCAAATGACTGGTCCGATTACTAATGCACAG |
| 43266 | AQSPNNTTGAQ | 43616 | GCGCAGTCGCCGAATAATACGACGGGGGCGCAG |
| 43267 | AQSSGGRTVAQ | 43617 | GCGCAGTCGTCGGGGGGGAGGACGGTGGCGCAG |
| 43268 | AQVNVTNQSAQ | 43618 | GCCCAAGTCAACGTCACGAACCAATCAGCCCAA |
| 43269 | AQTSGTAMAAQ | 43619 | GCCCAAACGTCAGGCACGGCCATGGCCGCCCAA |
| 43270 | AQSSMALSEAQ | 43620 | GCTCAGAGTAGTATGGCTCTTAGTGAGGCTCAG |
| 43271 | AQPVNTQRVAE | 43621 | GCGCAGCCGGTGAATACGCAGAGGGTGGCGGAG |
| 43272 | AQFVSGSHAAQ | 43622 | GCTCAGTTTGTTAGTGGTAGTCATGCTGCTCAG |
| 43273 | AQHKSMSRLAQ | 43623 | GCGCAGCATGAGTCGATGTCGAGGTTGGCGCAG |
| 43274 | AQSLGLTQQAQ | 43624 | GCTCAGAGTCTTGGTCTTACTCAGCAGGCTCAG |
| 43275 | AQHFDYSAPAQ | 43625 | GCGCAGCATTTTGATTATTCGGCGCCGGCGCAG |
| 43276 | AQDLSTSSRAQ | 43626 | GCACAAGACTTAAGCACTAGCAGCAGGGCACAA |
| 43277 | AQKNAITGPAQ | 43627 | GCCCAAAAGAACGCCCTTACGCGCCCCGCCCAA |
| 43278 | AQITQMAAQAQ | 43628 | GCCCAAATTACTCAGATGGCTGCGCAGGCACAG |
| 43279 | AQMNAPLMTAQ | 43629 | GCGCAGATGAATGCGCCGTTGATGACGGCGCAG |
| 43280 | AQYGKSNSLAQ | 43630 | GCACAATATGGAAAGAGCAACAGCTTAGCACAA |
| 43281 | AQHSTPGRAAQ | 43631 | GCCCAACATTCAACGCCCGGCCGAGCCGCCCAA |
| 43282 | AQLENARGTAQ | 43632 | GCCCAACTTGAGAATGCTCGGGGGACGGCACAG |
| 43283 | AQQTSTSNAAQ | 43633 | GCACAACAAACTAGCACTAGTAACGCAGCACAA |
| 43284 | AQSVLSVNAAQ | 43634 | GCCCAATCTGTGCTTTCGGTTAATGCTGCACAG |
| 43285 | AQAAVSSIPAQ | 43635 | GCTCAGGCTGCTGTTAGTAGTATTCCTGCTCAG |
| 43286 | AQVEDVRQLAQ | 43636 | GGCCAAGTCGAAGACGTCCGACAACTTGCCCAA |
| 43287 | AQVISGRDAAQ | 43637 | GCGCAGGTTATTAGTGGTCGTGATGCTGCTCAG |
| 43288 | AQTKSQEVSAQ | 43638 | GCCCAAACGAAGTCGCAGGAGGTTAGTGCACAG |
| 43289 | AQKPASIPNAQ | 43639 | GCCCAAAAGCCTGCGTCGATTCCTAATGCACAG |
| 43290 | AQYAVQSTHAQ | 43640 | GCGCAGTATGCGGTGCAGTCGACGCATGCGCAG |
| 43291 | AQSTRTVJPAQ | 43641 | GCACAAAGCACTAGGACTGTAATACCAGCACAA |
| 43292 | AQKATGLTSAQ | 43642 | GCTCAGAAGGCTACTGGTCTTACTAGTGCTCAG |
| 43293 | AQLTMQRSSAQ | 43643 | GCACAATTAACTAACCAAAGGAGCAGCGCACAA |
| 43294 | AQDHGKSGGAQ | 43644 | GCACAAGACCACGGAAAGAGCGGAGGAGCACAA |
| 43295 | AQVSMNSRGAQ | 43645 | GCCCAAGTTTCGATGAATTCTCGTGGGGCACAG |
| 43296 | AQQPSLRRDAQ | 43646 | GCCCAACAGCCTTCGCTTAGGCGGGATGCACAG |
| 43297 | AQRSNQNANAQ | 43647 | GCGCAGAGGTCGAATCAGAATGCGAATGCGCAG |
| 43298 | AQPGAGVREAQ | 43648 | GCCCAACCCGGCGCCGGCGTCCGAGAAGCCCAA |
| 43299 | aqtgrsdsnao | 43649 | GCCCAAACTGGGCGTTCTGATAGTAATGCACAG |
| 43300 | AQSNSALIAAQ | 43650 | GCGCAGTCGAATTCGGCGTTGATTGCGGCGCAG |
| 43301 | AQPIASVSPAQ | 43651 | GCGCAGCCGATTGCGTCGGTGTCGCCGGCGCAG |
| 43302 | AQHTFMTEVAQ | 43652 | GCTCAGCATACTACTAATACTGAGGTTGCTCAG |
| 43303 | AQQKSTYSPAQ | 43653 | GCCCAACAGAAGAGTACTTATAGTCCTGCACAG |
| 43304 | AQQTQHTGPAQ | 43654 | GCTCAGCAGACTCAGCATACTGGTCCTGCTCAG |
| 43305 | AQMERISIHAQ | 43655 | GCGCAGATGGAGAGGATTTCGATTCATGCGCAG |
| 43306 | AQDSGNKMNAQ | 43656 | GCTCAGGATAGTGGTAATAAGATGAATGCTCAG |
| 43307 | AQSAVHNGVAQ | 43657 | GCGCAGTCGGCGGTGCATAATGGGGTGGCGCAG |
| 43308 | AQSASLPMAAQ | 43658 | GCCCAATCAGCCTCACTTCCCATGGCCGCCCAA |
| 43309 | AQNLASTSPAQ | 43659 | GCCCAAAACCTTGCCTCAACGTCACCCGCCCAA |
| 43310 | AQVMHTLDAAQ | 43660 | GCTCAGGTTATGCATACTCTTGATGCTGCTCAG |
| 43311 | AQSQSGGDRAQ | 43661 | GCCCAAAGTCAGAGTGGGGGTGATAGGGCACAG |
| 43312 | AQVATIVRSAQ | 43662 | GCCCAAGTTGCGACTATTGTTCGTTCTGCACAG |
| 43313 | AQSPQRNSTAQ | 43663 | GCCCAATCGCCTCAGCGGAATTCTACGGCACAG |
| 43314 | AQEVSVNVAAQ | 43664 | GCCCAAGAAGTCTCAGTCAACGrCGCCGCCCAA |
| 43315 | AQ7GTPRNDAQ | 43665 | GCCCAAACGGGTACTCCGCGGAATGATGCACAG |
| 43316 | AQYTDNYGVAQ | 43666 | GCACAATATACTGACAACTATGGACTAGCACAA |
| 43317 | AQLDGSPARAQ | 43667 | GCGCAGTTGGATGGGTCGCCGGCGAGGGCGCAG |
| 43318 | AQNGSDASLAQ | 43668 | GCACAAAACGGAAGCGACGCAAGCTTAGCACAA |
| 43319 | AQNVGREQPAQ | 43669 | GCTCAGAATGTTGGTCGTGAGCAGCCTGCTCAG |
| 43320 | AQPPAGNTQAQ | 43670 | GCCCAACCGCCTGCGGGTAATACTCAGGCACAG |
| 43321 | AQVTPHFPSAQ | 43671 | GCTCAGGTTACTCCTCATTTTCCTAGTGCTCAG |
| 43322 | AQATHAASPAQ | 43672 | GCCCAAGGTACTCATGCTGCGAGTCCGGCACAG |
| 43323 | AQSNVKVLAAQ | 43673 | GCGCAATCAAACGTCAAGGTCCTTGCCGCCCAA |
| 43324 | AQNAAKAVLAQ | 43674 | GCCCAAAACGCCGCCAAGGCCGTCCTTGCCCAA |
| 43325 | AQTDKPHSQAQ | 43675 | GCACAAACTGACAAGGCACACAGCCAAGCACAA |
| 43326 | AQSATSNFLAQ | 43676 | GCCCAATCAGCCACGTCAAACTTTCTTCCCCAA |
| 43327 | AQNLRAEVVAQ | 43677 | GCGCAGAATTTGAGGGCGGAGGTGGTGGCGCAG |
| 43328 | AQNERSiESAQ | 43678 | GCCCAAAACGAACGATCAATCGAATCAGCCCAA |
| 43329 | AQYPNDANPAQ | 43679 | GCACAATATCCAAACGACGCAAACCCAGCACAA |
| 43330 | AQASLLSQPAQ | 43680 | GCCCAAGCCTCACTTCTTTCACAACCCGCCCAA |
| 43331 | AQEFNTRDAAQ | 43681 | GCACAAGAATTCAACACTAGGGACGCAGCACAA |
| 43332 | AQELVSSLGAQ | 43682 | GCCCAAGAGCTTGTGAGTAGTCTGGGGGCACAG |
| 43333 | AQSAQAYTEAQ | 43683 | GCCCAAAGTGCGCAGGCTTATACTGAGGCACAG |
| 43334 | AQMSTESHGAQ | 43684 | GCCCAAATGTCAACGGAATCACATGGCGCCCAA |
| 43335 | AQSQNMSNIAQ | 43685 | GCCCAATCACAAAACATGTCAAACATCGCCCAA |
| 43336 | AQVGNDNRSAQ | 43686 | GCCCAAGTGGGTAATGATAATCGGTCGGCACAG |
| 43337 | AQLQSSNNPAQ | 43687 | GCGCAGTTGCAGTCGTCGAATAATCCGGCGCAG |
| 43338 | AQDSAPLRSAQ | 43688 | GCTCAGGATAGTGCTCCTCTTCGTAGTGCTCAG |
| 43339 | AQMGGTIMSAQ | 43689 | GCCCAAATGGGCGGCACGATCATGTCAGCCCAA |
| 43340 | AQHYTSGAEAQ | 43690 | GCGCAGCATTATACGTCGGGGGCGGAGGCGCAG |
| 43341 | AQSVGMNYKAQ | 43691 | GCCCAATCAGTCGGCATGAACTATAAGGCCCAA |

In some embodiments, the targeting peptide may include amino acid at position 1 to amino acid at position 2 of SEQ ID NO: 43073-43341. In some embodiments, the targeting peptide may include amino acid at position 1 to amino acid at position 3 of SEQ ID NO: 43073-43341. In some embodiments, the targeting peptide may include amino acid at position 2 to amino acid at position 3 of SEQ ID NO: 43073-43341. In some embodiments, the targeting peptide may include amino acid at position 1 to amino acid at position 4 of SEQ ID NO: 43073-43341. In some embodiments, the targeting peptide may include amino acid at position 2 to amino acid at position 4 of SEQ ID NO: 43073-43341. In some embodiments, the targeting peptide may include amino acid at position 3 to amino acid at position 4 of SEQ ID NO: 43073-43341. In some embodiments, the targeting peptide may include amino acid at position 1 to amino acid at position 5 of SEQ ID NO: 43073-43341. In some embodiments, the targeting peptide may include amino acid at position 2 to amino acid at position 5 of SEQ ID NO: 43073-43341. In some embodiments, the targeting peptide may include amino acid at position 3 to amino acid at position 5 of SEQ ID NO: 43073-43341. In some embodiments, the targeting peptide may include amino acid at position 4 to amino acid at position 5 of SEQ ID NO: 43073-43341. In some embodiments, the targeting peptide may include amino acid at position 1 to amino acid at position 6 of SEQ ID NO: 43073-43341. In some embodiments, the targeting peptide may include amino acid at position 2 to amino acid at position 6 of SEQ ID NO: 43073-43341. In some embodiments, the targeting peptide may include amino acid at position 3 to amino acid at position 6 of SEQ ID NO: 43073-43341. In some embodiments, the targeting peptide may include amino acid at position 4 to amino acid at position 6 of SEQ ID NO: 43073-43341. In some embodiments, the targeting peptide may include amino acid at position 5 to amino acid at position 6 of SEQ ID NO: 43073-43341. In some embodiments, the targeting peptide may include amino acid at position 1 to amino acid at position 7 of SEQ ID NO: 43073-43341. In some embodiments, the targeting peptide may include amino acid at position 2 to amino acid at position 7 of SEQ ID NO: 43073-43341. In some embodiments, the targeting peptide may include amino acid at position 3 to amino acid at position 7 of SEQ ID NO: 43073-43341. In some embodiments, the targeting peptide may include amino acid at position 4 to amino acid at position 7 of SEQ ID NO: 43073-43341. In some embodiments, the targeting peptide may include amino acid at position 5 to amino acid at position 7 of SEQ ID NO: 43073-43341. In some embodiments, the targeting peptide may include amino acid at position 6 to amino acid at position 7 of SEQ ID NO: 43073-43341. In some embodiments, the targeting peptide may include amino acid at position 1 to amino acid at position 8 of SEQ ID NO: 43073-43341. In some embodiments, the targeting peptide may include amino acid at position 2 to amino acid at position 8 of SEQ ID NO: 43073-43341. In some embodiments, the targeting peptide may include amino acid at position 3 to amino acid at position 8 of SEQ ID NO: 43073-43341. In some embodiments, the targeting peptide may include amino acid at position 4 to amino acid at position 8 of SEQ ID NO: 43073-43341. In some embodiments, the targeting peptide may include amino acid at position 5 to amino acid at position 8 of SEQ ID NO: 43073-43341. In some embodiments, the targeting peptide may include amino acid at position 6 to amino acid at position 8 of SEQ ID NO: 43073-43341. In some embodiments, the targeting peptide may include amino acid at position 7 to amino acid at position 8 of SEQ ID NO: 43073-43341. In some embodiments, the targeting peptide may include amino acid at position 1 to amino acid at position 9 of SEQ ID NO: 43073-43341. In some embodiments, the targeting peptide may include amino acid at position 2 to amino acid at position 9 of SEQ ID NO: 43073-43341. In some embodiments, the targeting peptide may include amino acid at position 3 to amino acid at position 9 of SEQ ID NO: 43073-43341. In some embodiments, the targeting peptide may include amino acid at position 4 to amino acid at position 9 of SEQ ID NO: 43073-43341. In some embodiments, the targeting peptide may include amino acid at position 5 to amino acid at position 9 of SEQ ID NO: 43073-43341. In some embodiments, the targeting peptide may include amino acid at position 6 to amino acid at position 9 of SEQ ID NO: 43073-43341. In some embodiments, the targeting peptide may include amino acid at position 7 to amino acid at position 9 of SEQ ID NO: 43073-43341. In some embodiments, the targeting peptide may include amino acid at position 8 to amino acid at position 9 of SEQ ID NO: 43073-43341. In some embodiments, the targeting peptide may include amino acid at position 1 to amino acid at position 10 of SEQ ID NO: 43073-43341. In some embodiments, the targeting peptide may include amino acid at position 2 to amino acid at position 10 of SEQ ID NO: 43073-43341. In some embodiments, the targeting peptide may include amino acid at position 3 to amino acid at position 10 of SEQ ID NO: 43073-43341. In some embodiments, the targeting peptide may include amino acid at position 4 to amino acid at position 10 of SEQ ID NO: 43073-43341. In some embodiments, the targeting peptide may include amino acid at position 5 to amino acid at position 10 of SEQ ID NO: 43073-43341. In some embodiments, the targeting peptide may include amino acid at position 6 to amino acid at position 10 of SEQ ID NO: 43073-43341. In some embodiments, the targeting peptide may include amino acid at position 7 to amino acid at position 10 of SEQ ID NO: 43073-43341. In some embodiments, the targeting peptide may include amino acid at position 8 to amino acid at position 10 of SEQ ID NO: 43073-43341. In some embodiments, the targeting peptide may include amino acid at position 9 to amino acid at position 10 of SEQ ID NO: 43073-43341. In some embodiments, the targeting peptide may include amino acid at position 1 to amino acid at position 11 of SEQ ID NO: 43073-43341. In some embodiments, the targeting peptide may include amino acid at position 2 to amino acid at position 11 of SEQ ID NO: 43073-43341. In some embodiments, the targeting peptide may include amino acid at position 3 to amino acid at position 11 of SEQ ID NO: 43073-43341. In some embodiments, the targeting peptide may include amino acid at position 4 to amino acid at position 11 of SEQ ID NO: 43073-43341. In some embodiments, the targeting peptide may include amino acid at position 5 to amino acid at position 11 of SEQ ID NO: 43073-43341. In some embodiments, the targeting peptide may include amino acid at position 6 to amino acid at position 11 of SEQ ID NO: 43073-43341. In some embodiments, the targeting peptide may include amino acid at position 7 to amino acid at position 11 of SEQ ID NO: 43073-43341. In some embodiments, the targeting peptide may include amino acid at position 8 to amino acid at position 11 of SEQ ID NO: 43073-43341. In some embodiments, the targeting peptide may include amino acid at position 9 to amino acid at position 11 of SEQ ID NO: 43073-43341. In some embodiments, the targeting peptide may include amino acid at position 10 to amino acid at position 11 of SEQ ID NO: 43073-43341.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

### Use of Targeting Peptides in AAV Particles

Targeting peptides may be stand-alone peptides or may be inserted into or conjugated to a parent sequence. In certain embodiments, the targeting peptides are inserted into the capsid protein of an AAV particle.

One or more targeting peptides may be inserted into a parent AAV capsid sequence to generate the AAV particles of the disclosure.

Targeting peptides may be inserted into a parent AAV capsid sequence in any location that results in fully functional AAV particles. The targeting peptide may be inserted in VP1, VP2 and/or VP3. Numbering of the amino acid residues differs across AAV serotypes, and so the exact amino acid position of the targeting peptide insertion may not be critical. As used herein, amino acid positions of the parent AAV capsid sequence are described using AAV9 (SEQ ID NO: 2) as reference.

In certain embodiments, the targeting peptides are inserted in a hypervariable region of the AAV capsid sequence. Non-limiting examples of such hypervariable regions include Loop IV and Loop VIII of the parent AAV capsid. While not wishing to be bound by theory, these surface exposed loops are unstructured and poorly conserved, making them ideal regions for insertion of targeting peptides.

In certain embodiments, the targeting peptide is inserted into Loop IV. In another embodiment, the targeting peptide is used to replace a portion, or all of Loop IV. As a non-limiting example, addition of the targeting peptide to the parent AAV capsid sequence may result in the replacement or mutation of at least one amino acid of the parent AAV capsid.

In certain embodiments, the targeting peptide is inserted into Loop VIII. In another embodiment, the targeting peptide is used to replace a portion, or all of Loop VIII. As a non-limiting example, addition of the targeting peptide to the parent AAV capsid sequence may result in the replacement or mutation of at least one amino acid of the parent AAV capsid.

In certain embodiments, more than one targeting peptide is inserted into a parent AAV capsid sequence. As a non-limiting example, targeting peptides may be inserted at both Loop IV and Loop VIII in the same parent AAV capsid sequence.

Targeting peptides may be inserted at any amino acid position of the parent AAV capsid sequence, such as, but not limited to, between amino acids at positions 586-592, 588-589, 586-589, 452-458, 262-269, 464-473, 491-495, 546-557 and/or 659-668.

In a preferred embodiment, the targeting peptides are inserted into a parent AAV capsid sequence between amino acids at positions 588 and 589 (Loop VIII). In certain embodiments, the parent AAV capsid is AAV9 (SEQ ID NO: 2). In a second embodiment, the parent AAV capsid is K449R AAV9 (SEQ ID NO: 3).

The targeting peptides described herein may increase the transduction of the AAV particles of the disclosure to a target tissue as compared to the parent AAV particle lacking a targeting peptide insert. In certain embodiments, the targeting peptide increases the transduction of an AAV particle to a target tissue by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In certain embodiments, the targeting peptide increases the transduction of an AAV particle to a cell or tissue of the CNS by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In certain embodiments, the targeting peptide increases the transduction of an AAV particle to a cell or tissue of the PNS by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

In certain embodiments, the targeting peptide increases the transduction of an AAV particle to a cell or tissue of the DRG by at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 200%, 300%, 400%, 500%, or more as compared to a parent AAV particle lacking a targeting peptide insert.

### Viral Genome of the AAV Particle

AAV particles of the disclosure, comprising targeting peptides, may be used for the delivery of any viral genome to a target tissue (e.g., CNS and/or DRG). The viral genome may encode any payload, such as but not limited to a polypeptide, an antibody, an enzyme, an RNAi agent and/or components of a gene editing system. In certain embodiments, the AAV particles of the disclosure are used to deliver a payload to cells of the CNS, after intravenous delivery. In another embodiment, the AAV particles of the disclosure are used to deliver a payload to cells of the DRG, after intravenous delivery.

A viral genome of an AAV particle of the disclosure, comprises a nucleic acid sequence with at least one payload region encoding a payload, and at least one ITR. A viral genome typically comprises two ITR sequences, one at each of the 5′ and 3′ ends. Further, a viral genome of the AAV particles of the disclosure may comprise nucleic acid sequences for additional components, such as, but not limited to, a regulatory element (e.g., promoter), untranslated regions (UTR), a polyadenylation sequence (polyA), a filler or stuffer sequence, an intron, and/or a linker sequence for enhanced expression.

These viral genome components can be selected and/or engineered to further tailor the specificity and efficiency of expression of a given payload in a target tissue (e.g., CNS or DRG).

### Viral Genome Component: Inverted Terminal Repeats (ITRs)

The AAV particles of the present disclosure comprise a viral genome with at least one ITR and a payload region. In certain embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the disclosure may be comprised of naturally occurring polynucleotide sequences or recombinantly derived polynucleotide sequences.

The ITRs may be derived from the same serotype as the capsid, selected from any of the known serotypes, or a derivative thereof. The ITR may be of a different serotype than the capsid. In certain embodiments, the AAV particle has more than one ITR. In a non-limiting example, the AAV particle has a viral genome comprising two ITRs. In certain embodiments, the ITRs are of the same serotype as one another. In another embodiment, the ITRs are of different serotypes. Non-limiting examples include zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 105, 130, 140, 141, 142, 145 nucleotides in length. ITRs encompassed by the present disclosure include those with at least 90% identity, at least 95% identity, at least 98% identity, or at least 99% identity to a known AAV serotype ITR sequence.

### Viral Genome Component: Promoters

In certain embodiments, the payload region of the viral genome comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of a payload in a target cell may require a specific promoter, including but not limited to, a promoter that is species specific, inducible, tissue-specific, or cell cycle-specific (Parr et al., Nat. Med. 3:1145-9 (1997); the contents of which are herein incorporated by reference in their entirety).

In certain embodiments, the promoter is deemed to be efficient when it drives expression of the payload encoded by the viral genome of the AAV particle.

In certain embodiments, the promoter is a promoter deemed to be efficient when it drives expression in a cell being targeted.

In certain embodiments, the promoter is a promoter having a tropism for a cell being targeted.

In certain embodiments, the promoter drives expression of the payload for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter is a selected for sustained expression of a payload in tissues and/or cells of the central or peripheral nervous system.

Promoters may be naturally occurring or non-naturally occurring. Non-limiting examples of promoters include those derived from viruses, plants, mammals, or humans. In some embodiments, the promoters may be those derived from human cells or systems. In some embodiments, the promoter may be truncated or mutated.

Promoters which drive or promote expression in most tissues include, but are not limited to, the human elongation factor 1α-subunit (EF1α) promoter, the cytomegalovirus (CMV) immediate-early enhancer and/or promoter, the chicken β-actin (CBA) promoter and its derivative CAG, β glucuronidase (GUSB) promoter, or ubiquitin C (UBC) promoter. Tissue-specific promoters can be used to restrict expression to certain cell types such as, but not limited to, cells of the central or peripheral nervous systems, targeted regions within (e.g., frontal cortex), and/or sub-sets of cells therein (e.g., excitatory neurons). As non-limiting examples, cell-type specific promoters may be used to restrict expression of a payload to excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, astrocytes, oligodendrocytes, and/or Schwann cells.

Cell-type specific promoters also exist for other tissues of the body, with non-limiting examples including, liver promoters (e.g., hAAT, TBG), skeletal muscle specific promoters (e.g., desmin, MCK, C512), B cell promoters, monocyte promoters, leukocyte promoters, macrophage promoters, pancreatic acinar cell promoters, endothelial cell promoters, lung tissue promoters, and/or cardiac or cardiovascular promoters (e.g., αMHC, cTnT, and CMV-MLC2k).

Non-limiting examples of tissue-specific promoters for targeting payload expression to central nervous system tissues and cells include synapsin (Syn), glutamate vesicular transporter (VGLUT), vesicular GABA transporter (VGAT), parvalbumin (PV), sodium channel Naᵥ 1.8 (SCN10A), tyrosine hydroxylase (TH), choline acetyltransferase (ChaT), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), neuron-specific enolase (NSE). β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In certain embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 nucleotides.

In certain embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 nucleotides. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 nucleotides. In certain embodiments, the promoter is a combination of a 382 nucleotide CMV-enhancer sequence and a 260 nucleotide CBA-promoter sequence.

In certain embodiments, the viral genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, EFIα, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015, 2(2): ENEURO.0001-15; the contents of which are herein incorporated by reference in their entirety) evaluated the expression of eGFP in AAV8 with CMV and UBC promoters and AAV2 with the CMV promoter after injection in the motor cortex. Intranasal administration of a plasmid containing a UBC or EFIα promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009, 16(7): 927-932; the contents of which are herein incorporated by reference in their entirety) evaluated an HOH construct with a hGUSB promoter, a HSV-1LAT promoter and an NSE promoter and found that the HpH construct showed weaker expression than NSE in mouse brain. Passini and Wolfe (J. Virol. 2001, 12382-12392, the contents of which are herein incorporated by reference in their entirety) evaluated the long term effects of the HpH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650-nucleotide promoter and NFH is a 920 nucleotide promoter which are both absent in the liver but NFH is abundant in the sensory proprioceptive neurons, brain and spinal cord and NFH is present in the heart. SCN8A (Nav 1.6) is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A. Mamm Genome (2007) 18:723-731; and Raymond et al. Expression of Alternatively Spliced Sodium Channel α-subunit genes. Journal of Biological Chemistry (2004) 279(44) 46234-46241; the contents of each of which are herein incorporated by reference in their entireties).

Any of the promoters taught by the aforementioned Yu, Soderblom, Gill, Husain, Passini, Xu, Drews or Raymond may be used in the present disclosures.

In certain embodiments, the promoter is not cell specific.

In certain embodiments, the promoter is a RNA pol 111 promoter. As a non-limiting example, the RNA pol III promoter is U6. As a non-limiting example, the RNA pol III promoter is H1.

In certain embodiments, the viral genome comprises an enhancer element.

In certain embodiments, the viral genome comprises an engineered promoter.

In another embodiment, the viral genome comprises a promoter from a naturally expressed protein.

### Viral Genome Component: Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue or DRG) may be engineered into UTRs to enhance stability and protein production. As a non-limiting example, a 5′ UTR from mRNA normally expressed in the brain (e.g., huntingtin) may be used in the viral genomes of the AAV particles of the disclosure to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In certain embodiments, the 5′UTR in the viral genome includes a Kozak sequence.

In certain embodiments, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class 11 AREs, such as, but not limited to, GM-CSF and TNF-a, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise. AREs can be identified and removed or mutated to increase the intracellular stability and thus increase translation and production of the resultant protein.

In certain embodiments, the 3′ UTR of the viral genome may include an oligo(dT) sequence for templated addition of a poly-A tail.

In certain embodiments, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which has perfect Watson-Crick sequence complementarity to the miRNA target sequence of the nucleic acid.

In certain embodiments, the viral genome may be engineered to include, alter or remove at least one miRNA binding site, full sequence or seed region.

Any UTR from any gene known in the art may be incorporated into the viral genome of the AAV particle. These UTRs, or portions thereof, may be placed in the same orientation as in the gene from which they were selected or they may be altered in orientation or location. In certain embodiments, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In certain embodiments, the viral genome of the AAV particle comprises at least one artificial UTR which is not a variant of a wild type UTR.

In certain embodiments, the viral genome of the AAV particle comprises UTRs which have been selected from a family of transcripts whose proteins share a common function, structure, feature or property.

### Viral Genome Component: Polyadenylation Sequence

The viral genome of the AAV particles of the present disclosure may comprise at least one polyadenylation sequence. In certain embodiments, the viral genome of the AAV particle comprises a polyadenylation sequence between the 3′ end of the payload encoding region and the 5′ end of the 3′ITR.

In certain embodiments, the polyadenylation sequence or “polyA sequence” may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441,442, 443, 444, 445, 446, 447, 448, 449, 450,451, 452, 453, 454, 455, 456, 457,458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 nucleotides in length.

In certain embodiments, the polyadenylation sequence is 50-100 nucleotides in length.

In certain embodiments, the polyadenylation sequence is 50-150 nucleotides in length.

In certain embodiments, the polyadenylation sequence is 50-160 nucleotides in length.

In certain embodiments, the polyadenylation sequence is 50-200 nucleotides in length.

In certain embodiments, the polyadenylation sequence is 60-100 nucleotides in length.

In certain embodiments, the polyadenylation sequence is 60-150 nucleotides in length.

In certain embodiments, the polyadenylation sequence is 60-160 nucleotides in length.

In certain embodiments, the polyadenylation sequence is 60-200 nucleotides in length.

In certain embodiments, the polyadenylation sequence is 70-100 nucleotides in length.

In certain embodiments, the polyadenylation sequence is 70-150 nucleotides in length.

In certain embodiments, the polyadenylation sequence is 70-160 nucleotides in length.

In certain embodiments, the polyadenylation sequence is 70-200 nucleotides in length.

In certain embodiments, the polyadenylation sequence is 80-100 nucleotides in length.

In certain embodiments, the polyadenylation sequence is 80-150 nucleotides in length.

In certain embodiments, the polyadenylation sequence is 80-160 nucleotides in length.

In certain embodiments, the polyadenylation sequence is 80-200 nucleotides in length.

In certain embodiments, the polyadenylation sequence is 90-100 nucleotides in length.

In certain embodiments, the polyadenylation sequence is 90-150 nucleotides in length.

In certain embodiments, the polyadenylation sequence is 90-160 nucleotides in length.

In certain embodiments, the polyadenylation sequence is 90-200 nucleotides in length.

### Viral Genome Component: Introns

In certain embodiments, the viral genome of the AAV particles of the present disclosure comprises at least one element to enhance the payload target specificity and expression (See e.g., Powell et al. Viral Expression Cassette Elements to Enhance Transgene Target Specificity and Expression in Gene Therapy, Discov. Med, 2015, 19(102): 49-57; the contents of which are herein incorporated by reference in their entirety) such as an intron. Non-limiting examples of introns include, MVM (67-97 bps), FIX truncated intron 1 (300 bps), f-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In certain embodiments, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nucleotides. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides.

### Viral Genome Component: Stuffer Sequences

In certain embodiments, the viral genome of the AAV particles of the present disclosure comprises at least one element to improve packaging efficiency and expression, such as a stuffer or filler sequence. Non-limiting examples of stuffer sequences include albumin and/or alpha-1 antitrypsin. Any known viral, mammalian, or plant sequence may be manipulated for use as a stuffer sequence.

In certain embodiments, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 nucleotides.

Viral Genome Component: miRNA

In certain embodiments, the viral genome comprises at least one sequence encoding a miRNA to reduce the expression of the payload in an “off-target” tissue. As used herein, “off-target” indicates a tissue or cell-type unintentionally targeted by the AAV particles of the disclosure. As an example, an “off-target” tissue or cell when targeting the DRG, may be neurons of other ganglia, such as those of the sympathetic or parasympathetic nervous system, miRNAs and their targeted tissues are well known in the art. As a non-limiting example, a miR-122 miRNA may be encoded in the viral genome to reduce the expression of the viral genome in the liver.

### Viral Genome Component: Selectable Marker

In some embodiments, the viral genome of the AAV particles of the disclosure optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes are described in International Publication Nos. WO 1996023810 and WO 1996030540; Heim et al., Current Biology 2:178-182 (1996); Heim et al., Proc. Natl. Acad. Sci. USA (1995); or Heim et al., Science 373:663-664 (1995), the contents of each of which are incorporated herein by reference in their entirety.

### Genome Size

In certain embodiments, the AAV particles of the disclosure may comprise a single-stranded or double-stranded viral genome. The size of the viral genome may be small, medium, large or the maximum size. As described above, the viral genome may comprise a promoter and a polyA tail.

In certain embodiments, the viral genome may be a small single stranded viral genome. A small single stranded viral genome may be 2.1 to 3.5 kb in size such as, but not limited to, about 2.1, 2.2, 2.3, 2.4, 2.5, 2.6, 2.7, 2.8, 2.9, 3.0, 3.1, 3.2, 3.3, 3.4, and 3.5 kb in size.

In certain embodiments, the viral genome may be a small double stranded viral genome. A small double stranded viral genome may be 1.3 to 1.7 kb in size such as, but not limited to, about 1.3, 1.4, 1.5, 1.6, and 1.7 kb in size.

In certain embodiments, the viral genome may be a medium single stranded viral genome. A medium single stranded viral genome may be 3.6 to 4.3 kb in size such as, but not limited to, about 3.6, 3.7, 3.8, 3.9, 4.0, 4.1, 4.2 and 4.3 kb in size.

In certain embodiments, the viral genome may be a medium double stranded viral genome. A medium double stranded viral genome may be 1.8 to 2.1 kb in size such as, but not limited to, about 1.8, 1.9, 2.0, and 2.1 kb in size.

In certain embodiments, the viral genome may be a large single stranded viral genome. A large single stranded viral genome may be 4.4 to 6.0 kb in size such as, but not limited to, about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size.

In certain embodiments, the viral genome may be a large double stranded viral genome. A large double stranded viral genome may be 2.2 to 3.0 kb in size such as, but not limited to, about 2.2, 2.3, 2.4, 2.5, 2.6, 2.7, 2.8, 2.9 and 3.0 kb in size.

### Payloads

The AAV particles of the present disclosure comprise a viral genome with at least one payload region. As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

The payload region may comprise a combination of coding and non-coding nucleic acid sequences.

In some embodiments, the payload region may encode a coding or non-coding RNA.

In certain embodiments, the AAV particle comprises a viral genome with a payload region encoding more than one payload of interest. In such an embodiment, a viral genome encoding more than one payload may be replicated and packaged into a viral particle. A target cell transduced with a viral particle comprising more than one payload may express each of the payloads in a single cell.

### Polypeptides

Where the payload region encodes a polypeptide, the polypeptide may be a peptide or protein. As a non-limiting example, the payload region may encode at least one allele of apolipoprotein E (APOE) such as, but not limited to ApoE2. ApoE3 and/or ApoE4. In certain embodiments, the payload region encodes ApoE2 (cys112, cys158). In certain embodiments, the payload region encodes ApoE3 (cys112, arg158). In certain embodiments, the payload region of the encodes ApoE4 (arg112, arg158). As a second non-limiting example, the payload region may encode a human or a primate frataxin protein, or fragment or variant thereof. As another non-limiting example, the payload region may encode an antibody, or a fragment thereof. As another non-limiting example, the payload region may encode human aromatic L-amino acid decarboxylase (AADC), or a fragment or variant thereof. As another non-limiting example, the payload region may encode human survival of motor neuron (SMN) 1 or SMN2, or fragments or variants thereof. As another non-limiting example, the payload region may encode frataxin (FXN). As another non-limiting example, the payload region may encode APOE (APOE2, APOE3, APOE4), or fragments or variants thereof. As another non-limiting example, the payload region may encode glucocerebrosidase (GBA1), or a fragment or variant thereof. As another non-limiting example, the payload region may encode granulin precursor or progranulin (GRN), or a fragment or variant thereof. As another non-limiting example, the payload region may encode aspartoacylase (ASPA), or a fragment or variant thereof. As another non-limiting example, the payload region may encode tripeptidyl peptidase I (CLN2), or a fragment or variant thereof. As another non-limiting example, the payload region may encode beta-galactosidase (GLB1), or a fragment or variant thereof. As another non-limiting example, the payload region may encode N-sulphoglucosamine sulphohydrolase (SGSH), or a fragment or variant thereof. As another non-limiting example, the payload region may encode N-acetyl-alpha-glucosaminidase (NAGLU), or a fragment or variant thereof. As another non-limiting example, the payload region may encode iduronate 2-sulfatase (IDS), or a fragment or variant thereof. As another non-limiting example, the payload region may encode Intracellular cholesterol transporter (NPC1), or a fragment or variant thereof. As another non-limiting example, the payload region may encode gigaxonin (GAN), or a fragment or variant thereof. The AAV viral genomes encoding polypeptides described herein may be useful in the fields of human disease, viruses, infections veterinary applications and a variety of in vivo and in vitro settings.

Amino acid sequences encoded by payload regions of the viral genomes of the disclosure may be translated as a whole polypeptide, a plurality of polypeptides or fragments of polypeptides, which independently may be encoded by one or more nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

### Antibodies

Where the payload region encodes an antibody, the “antibody” may be an antibody, a fragment, or any derivative thereof, which may contribute to the formation of a “functional antibody”, exhibiting the desired biological activity. As non-limiting examples, an antibody may be a native antibody (e.g., with two heavy and two light chains), a heavy chain variable region, a light chain variable region, a heavy chain constant region, a light chain constant region, Fab, Fab′, F(ab′)₂, Fv, or scFv fragments, a diabody, a linear antibody, a single-chain antibody, a multi-specific antibody, an intrabody, one or more heavy chain complementarity determining regions (CDR), one or more light chain CDRs, a bi-specific antibody, a monoclonal antibody, a polyclonal antibody, a humanized antibody, an antibody mimetic, an antibody variant, a miniaturized antibody, a unibody, a maxibody, and/or a chimeric antigen receptor.

As used herein, “antibody-based” or “antibody-derived” compositions are monomeric or multi-meric polypeptides which comprise at least one amino-acid region derived from a known or parental antibody sequence and at least one amino acid region derived from a non-antibody sequence, e.g., mammalian protein.

Payload regions may encode polypeptides that form or function as any antibody, including antibodies that are known in the art and/or antibodies that are commercially available. The encoded antibodies may be therapeutic, diagnostic, or for research purposes. The encoded antibodies may be useful in the treatment of neurological disease or any disorders associated with the central and/or peripheral nervous systems.

In certain embodiments, the viral genome of the AAV particle may comprise nucleic acids which have been engineered to enable or enhance the expression of antibodies, antibody fragments, or components thereof.

Antibodies encoded in payload regions of the AAV particles of the present disclosure may be, but are not limited to, antibodies targeting β-amyloid, APOE, tau, SOD1. TDP43, huntingtin, and/or synuclein.

### RNAi Agents

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. RNAi mediated gene silencing can specifically inhibit targeted gene expression. Where the payload region of the viral genome of the AAV particles of the present disclosure encodes an RNAi agent, the RNAi agent may be, but is not limited to, dsRNA, siRNA, shRNA, pre-miRNA, pri-miRNA, miRNA, stRNA, lncRNA, piRNA, or snoRNA. Non-limiting examples of a target gene of an RNAi agent include, SOD1, MAPT. APOE, HTT, C9ORF72, TDP-43, APP, BACE, SNCA, ATXN1, ATXN2, ATXN3, ATXN7, SCN1A-SCN5A, or SCN8A-SCN11A.

The AAV particles of the present disclosure may comprise viral genomes encoding RNAi agents, wherein the RNAi agent targets the mRNA of a gene of interest to interfere with gene expression and/or protein production. Such AAV particles may be used as a therapeutic, a diagnostic, or for research purposes.

In certain embodiments, the RNAi agent may target the gene of interest along any segment of their respective nucleotide sequence.

In certain embodiments, the RNAi agent may target the gene of interest at the location of a single-nucleotide polymorphism (SNP) or variant within the nucleotide sequence.

In some embodiments, a nucleic acid sequence encoding an RNAi agent, or a single strand of an RNAi agent, is inserted into the viral genome of the AAV particle and introduced into cells, specifically cells in the central nervous system or cells of the DRG.

The RNAi agent may be an siRNA duplex, wherein the siRNA duplex contains an antisense strand (guide strand) and a sense strand (passenger strand) hybridized together forming a duplex structure, wherein the antisense strand is complementary to the nucleic acid sequence of the targeted gene, and wherein the sense strand is homologous to the nucleic acid sequence of the targeted gene. In some aspects, the 5′ end of the antisense strand has a 5′ phosphate group and the 3′ end of the sense strand contains a 3′ hydroxyl group. In other aspects, there are none, one or 2 nucleotide overhangs at the 3′ end of each strand.

Each strand of an siRNA duplex targeting a gene of interest may be about 19 to 25, 19 to 24 or 19 to 21 nucleotides in length, preferably about 19 nucleotides, 20 nucleotides, 21 nucleotides, 22 nucleotides, 23 nucleotides, 24 nucleotides, or 25 nucleotides in length.

In certain embodiments, an siRNA or dsRNA includes at least two sequences that are complementary to each other. The dsRNA includes a sense strand having a first sequence and an antisense strand having a second sequence. The antisense strand includes a nucleotide sequence that is substantially complementary to at least part of an mRNA encoding the target gene, and the region of complementarity is 30 nucleotides or less, and at least 15 nucleotides in length. Generally, the dsRNA is 19 to 25, 19 to 24 or 19 to 21 nucleotides in length. In some embodiments, the dsRNA is from about 15 to about 25 nucleotides in length, and in other embodiments the dsRNA is from about 25 to about 30 nucleotides in length. In some embodiments, the dsRNA is about 15 nucleotides in length, 16 nucleotides in length, 17 nucleotides in length, 18 nucleotides in length, 19 nucleotides, 20 nucleotides, 21 nucleotides, 22 nucleotides, 23 nucleotides, 24 nucleotides, 25 nucleotides in length, 26 nucleotides in length, 27 nucleotides in length, 28 nucleotides in length, 29 nucleotides in length, or 30 nucleotides in length.

The dsRNA, whether directly administered or encoded in a viral genome in an AAV particle, upon contacting with a cell expressing the target protein, inhibits the expression of the protein by at least 10%, at least 20%, at least 25%, at least 30%, at least 35% or at least 40% or more, such as when assayed by a method known in the art.

In certain embodiments, the RNAi agent may be used to reduce the expression of target protein by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of target protein expression may be reduced 50-90%.

In certain embodiments, the RNAi agent may be used to reduce the expression of target mRNA by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of target mRNA expression may be reduced 50-90%.

In certain embodiments, RNAi agent may be used to reduce the expression of target protein and/or mRNA in at least one region of the CNS. The expression of target protein and/or mRNA is reduced by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in at least one region of the CNS. As a non-limiting example, the expression of target protein and mRNA in neurons (e.g., cortical neurons) is reduced by 50-90%. As a non-limiting example, the expression of target protein and mRNA in neurons (e.g., cortical neurons) is reduced by 40-50%.

In some embodiments, the AAV particle of the present disclosure comprising a viral genome encoding at least one RNAi agent targeting a gene of interest is administered to a subject in need for treating and/or ameliorating a disease, e.g., a neurological disorder of any disease associated with the central or peripheral nervous systems.

In certain embodiments, the RNAi agent is an siRNA.

Design of siRNA

AAV particles of the present disclosure may comprise a viral genome encoding one or more siRNA molecules (e.g., siRNA duplexes or encoded dsRNA) that are specifically designed to target a gene of interest and suppress target gene expression and protein production. In some aspects, the siRNA molecules are designed and used to selectively “knock out” target gene variants in cells, i.e., transcripts that are identified in neurological disease. In some aspects, the siRNA molecules are designed and used to selectively “knock down” target gene variants in cells.

In some embodiments, siRNA molecules targeting a gene of interest may be designed using any available design tools.

Some guidelines for designing siRNAs (for insertion into a viral genome of the AAV particles of the disclosure) have been proposed in the art. These guidelines generally recommend generating a 19-nucleotide duplexed region, symmetric 2-3 nucleotide 3′ overhangs, 5-phosphate and 3-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such considerations, together with the specific sequence of a target gene, highly effective siRNA molecules essential for suppressing mammalian target gene expression may be readily designed.

In certain embodiments, the sense and/or antisense strand is designed based on the method and rules outlined in European Patent Publication No. EP1752536, the contents of which are herein incorporated by reference in their entirety. As a non-limiting example, the 3′-terminal base of the sequence is adenine, thymine or uracil. As a non-limiting example, the 5′-terminal base of the sequence is guanine or cytosine. As a non-limiting example, the 3′-terminal sequence comprises seven bases rich in one or more bases of adenine, thymine and uracil.

In certain embodiments, an siRNA molecule comprises a sense strand and a complementary antisense strand in which both strands are hybridized together to form a duplex structure. The antisense strand has sufficient complementarity to the target mRNA sequence to direct target-specific RNAi, i.e., the siRNA molecule has a sequence sufficient to trigger the destruction of the target mRNA by the RNAi machinery or process.

In some embodiments, the antisense strand and target mRNA sequences have 100% complementarity. The antisense strand may be complementary to any part of the target mRNA sequence. Neither the identity of the sense sequence nor the homology of the antisense sequence need be 100% complementary to the target.

In other embodiments, the antisense strand and target mRNA sequences comprise at least one mismatch. As a non-limiting example, the antisense strand and the target mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

The siRNA molecule may have a length from about 10-50 or more nucleotides, i.e., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In certain embodiments, the siRNA molecule has a length from about 19 to 25, 19 to 24 or 19 to 21 nucleotides.

In some embodiments, the siRNA molecule can be a synthetic RNA duplex comprising about 19 nucleotides to about 25 nucleotides, and two overhanging nucleotides at the 3′-end.

The siRNA molecule may comprise an antisense sequence and a sense sequence, or a fragment or variant thereof. As a non-limiting example, the antisense sequence and the sense sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

The sense and antisense sequences may be completely complementary across a substantial portion of their length. In other embodiments, the sense sequence and antisense sequence may be at least 70, 80, 90, 95 or 99% complementary across independently at least 50, 60, 70, 80, 85, 90, 95, or 99% of the length of the strands.

In some embodiments, the sense and antisense strands of a siRNA duplex are linked by a short spacer sequence leading to the expression of a stem-loop structure termed short hairpin RNA (shRNA). The hairpin is recognized and cleaved by Dicer, thus generating mature siRNA molecules.

In some embodiments, the siRNA molecules, as well as associated spacer and/or flanking regions once designed, can be encoded by the viral genome of the AAV particles of the present disclosure, for delivery to a cell.

### Molecular Scaffold

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) includes a molecular scaffold which comprises at least one 5′ flanking sequence which may be of any length and may be derived in whole or in part from wild type microRNA sequence or be completely artificial. A 3′ flanking sequence may mirror the 5′ flanking sequence in size and origin. Either flanking sequence may be absent. In certain embodiments, both the 5′ and 3′ flanking sequences are absent. The 3′ flanking sequence may optionally contain one or more CNNC motifs, where “N” represents any nucleotide.

In some embodiments the 5′ and 3′ flanking sequences are the same length.

In some embodiments the 5′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 5′ flanking sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In certain embodiments, the molecular scaffold comprises at least one 3′ flanking region. As a non-limiting example, the 3′ flanking region may comprise a 3′ flanking sequence which may be of any length and may be derived in whole or in part from wild type microRNA sequence or be a completely artificial sequence.

In some embodiments the 3′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, the 3′ flanking sequence may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In some embodiments the 5′ and 3′ flanking sequences are the same sequence. In some embodiments they differ by 2%, 3%, 4%, 5%, 10%, 20% or more than 30% when aligned to each other.

Forming the stem of a stem loop structure is a minimum of at least one payload sequence. In some embodiments, the payload sequence comprises at least one nucleic acid sequence which is in part complementary or will hybridize to the target sequence. In some embodiments, the payload is an siRNA molecule or fragment of an siRNA molecule.

In some embodiments, the 5′ arm of the stem loop comprises a sense sequence.

In some embodiments, the 3′ arm of the stem loop comprises an antisense sequence. The antisense sequence, in some instances, comprises a “G” nucleotide at the 5′ most end.

In other embodiments, the sense sequence may reside on the 3′ arm while the antisense sequence resides on the 5′ arm of the stem of the stem loop structure.

Separating the sense and antisense sequence of the stem loop structure is a loop (also known as a loop motif). The loop may be of any length, between 4-30 nucleotides, between 4-20 nucleotides, between 4-15 nucleotides, between 5-15 nucleotides, between 6-12 nucleotides, 6 nucleotides, 7, nucleotides, 8 nucleotides, 9 nucleotides, 10 nucleotides, 11 nucleotides, and/or 12 nucleotides.

In some embodiments, the loop comprises at least one UGUG motif. In some embodiments, the UGUG motif is located at the 5′ terminus of the loop.

Spacer regions may be present in the modulatory polynucleotide to separate one or more modules from one another. There may be one or more such spacer regions present.

In certain embodiments, a spacer region of between 8-20, i.e., 8, 9, 10, 11, 12, 13, 14, 15, 16, 17, 18, 19, or 20 nucleotides may be present between the sense sequence and a flanking sequence.

In certain embodiments, the spacer is 13 nucleotides and is located between the 5′ terminus of the sense sequence and a flanking sequence. In certain embodiments, a spacer is of sufficient length to form approximately one helical turn of the sequence.

In certain embodiments, a spacer region of between 8-20. i.e., 8, 9, 10, 11, 12, 13, 14, 15, 16, 17, 18, 19, or 20 nucleotides may be present between the antisense sequence and a flanking sequence.

In certain embodiments, the spacer sequence is between 10-13, i.e., 10, 11, 12 or 13 nucleotides and is located between the 3′ terminus of the antisense sequence and a flanking sequence. In certain embodiments, a spacer is of sufficient length to form approximately one helical turn of the sequence.

In certain embodiments, the modulatory polynucleotide comprises in the 5′ to 3′ direction, a 5′ flanking sequence, a 5′ arm, a loop motif, a 3′ arm and a 3′ flanking sequence. As a non-limiting example, the 5′ arm may comprise a sense sequence and the 3′ arm comprises the antisense sequence. In another non-limiting example, the 5′ arm comprises the antisense sequence and the 3′ arm comprises the sense sequence.

In certain embodiments, the 5′ arm, payload (e.g., sense and/or antisense sequence), loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In certain embodiments, the molecular scaffold of the modulatory polynucleotides is aligned in order to have the rate of excision of the guide or antisense strand be greater than the rate of excision of the passenger or sense strand. The rate of excision of the guide or passenger strand may be, independently, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%.

In certain embodiments, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In certain embodiments, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In certain embodiments, the efficiency of the excision of the guide strand is greater than the excision of the passenger strand from the molecular scaffold. The excision of the guide strand may be 2, 3, 4, 5, 6, 7, 8, 9, 10 or more than 10 times more efficient than the excision of the passenger strand from the molecular scaffold.

In certain embodiments, the molecular scaffold comprises a dual-function targeting modulatory polynucleotide. As used herein, a “dual-function targeting” modulatory polynucleotide is a polynucleotide where both the guide and passenger strands knock down the same target or the guide and passenger strands knock down different targets.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

### AAV Production

Viral production disclosed herein describes processes and methods for producing AAV particles (with enhanced, improved and/or increased tropism for a target tissue) that may be used to contact a target cell to deliver a payload.

The present disclosure provides methods for the generation of AAV particles comprising targeting peptides. In certain embodiments, the AAV particles are prepared by viral genome replication in a viral replication cell. Any method known in the art may be used for the preparation of AAV particles. In certain embodiments, AAV particles are produced in mammalian cells (e.g., HEK293). In another embodiment, AAV particles are produced in insect cells (e.g., Sf9)

Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353 and WO2001023597; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. In certain embodiments, the AAV particles are made using the methods described in International Patent Publication WO2015191508, the contents of which are herein incorporated by reference in their entirety.

The viral replication cell may be selected from any biological organism, including prokaryotic (e.g., bacterial) cells, and eukaryotic cells, including, insect cells, yeast cells and mammalian cells. Viral replication cells commonly used for production of recombinant AAV viral particles include, but are not limited to, HEK293 cells, COS cells, HeLa cells, KB cells, and other mammalian cell lines as described in U.S. Pat. Nos. 6,156,303, 5,387,484, 5,741,683, 5,691,176, and U.S. Pat. No. 5,688,676; U.S. Patent Application Publication No. 2002/0081721, and International Patent Publication Nos. WO 2000047757, WO 2000024916, and WO 1996017947, the contents of each of which are herein incorporated by reference in their entirety. Viral replication cells may comprise other mammalian cells such as A549, WEH1, 3T3, 10T1/2, BHK, MDCK, COS 1, COS 7, BSC 1, BSC 40, BMT 10, VERO, W138, Saos, C2C12, L cells, HT1080, HepG2 and primary fibroblast, hepatocyte and myoblast cells derived from mammals. Viral replication cells may comprise cells derived from mammalian species including, but not limited to, human, monkey, mouse, rat, rabbit, and hamster. Viral replication cells may comprise cells derived from a cell type, including but not limited to fibroblast, hepatocyte, tumor cell, cell line transformed cell, etc.

In some embodiments, the present disclosure provides a method for producing an AAV particle in mammalian cells, comprising the steps of 1) simultaneously co-transfecting mammalian cells, such as, but not limited to HEK293 cells, with a viral genome comprising a payload region (payload construct), a viral genome comprising polynucleotide sequences for rep and cap genes (rep/cap construct) and a viral genome comprising polynucleotide sequences encoding helper components (helper construct), 2) harvesting and purifying the AAV particles comprising a viral genome. This triple transfection method of AAV particle production may be utilized to produce small lots of virus.

In certain embodiments, the AAV particles may be produced in a viral replication cell that comprises an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present disclosure. Cell lines may be used from Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which is herein incorporated by reference in its entirety.

In some embodiments, the present disclosure provides a method for producing an AAV particle in a baculovirus/Sf9 system, comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, and 5) harvesting and purifying AAV particles comprising a viral genome.

Briefly, the viral construct vector and the AAV payload construct vector are each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses may be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al., J Virol. 2006 February; 80 (4):1874-85, the contents of which are herein incorporated by reference in their entirety.

Production of AAV particles with baculovirus in an insect cell system may address known baculovirus genetic and physical instability. In certain embodiments, the production system addresses baculovirus instability over multiple passages by utilizing a titerless infected-cells preservation and scale-up system. Small scale seed cultures of viral producing cells are transfected with viral expression constructs encoding the structural, non-structural, components of the viral particle. Baculovirus-infected viral producing cells are harvested into aliquots that may be cryopreserved in liquid nitrogen; the aliquots retain viability and infectivity for infection of large scale viral producing cell culture Wasilko D J et al., Protein Expr Purif. 2009 June; 65(2):122-32, the contents of which are herein incorporated by reference in their entirety.

A genetically stable baculovirus may be used as the source of one or more of the components for producing AAV particles in invertebrate cells. In certain embodiments, defective baculovirus expression vectors may be maintained episomally in insect cells. In such an embodiment the bacmid vector is engineered with replication control elements, including but not limited to promoters, enhancers, and/or cell-cycle regulated replication elements.

In certain embodiments, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

AAV particles described herein may be produced by triple transfection or baculovirus mediated virus production, or any other method known in the art. Any suitable permissive or packaging cell known in the art may be employed to produce the particles. Mammalian cells are often preferred. Also preferred are trans-complementing packaging cell lines that provide functions deleted from a replication-defective helper virus, e.g., 293 cells or other E1a trans-complementing cells. A packaging cell line may be used that is stably transformed to express cap and/or rep genes. Alternatively, a packaging cell line may be used that is stably transformed to express helper constructs necessary for AAV particle assembly.

Recombinant AAV virus particles are, in some cases, produced and purified from culture supernatants according to the procedure as described in US20160032254, the contents of which are incorporated by reference.

In certain embodiments, AAV particles are produced wherein all three VP proteins are expressed at a stoichiometry around 1:1:10 (VP1:VP2:VP3). While not wishing to be bound by theory, the regulatory mechanisms that allow this controlled level of expression include the production of two mRNAs, one for VP1, and the other for VP2 and VP3, produced by differential splicing.

In certain embodiments, the viral construct vector(s) used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1. VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In certain embodiments, the viral construct vector(s) used for AAV production may contain a nucleotide sequence encoding the AAV rep proteins where the initiation codon of the AAV rep protein or proteins is a non-ATG. In certain embodiments, a single coding sequence is used for the Rep78 and Rep52 proteins, wherein initiation codon for translation of the Rep78 protein is a suboptimal initiation codon, selected from the group consisting of ACG, TTG, CTG and GTG, that effects partial exon skipping upon expression in insect cells, as described in U.S. Pat. No. 8,512,981, the contents of which is herein incorporated by reference in its entirety, for example to promote less abundant expression of Rep78 as compared to Rep52, which may be advantageous in that it promotes high vector yields.

### Small-Scale Production

In some cases, 293T cells (adhesion/suspension) are transfected with polyethylenimine (PEI) with plasmids required for production of AAV, i.e., AAV2 rep, an adenoviral helper construct and a ITR flanked payload cassette. The AAV2 rep plasmid also contains the cap sequence of the particular virus being studied. Twenty-four hours after transfection (no medium changes for suspension), which occurs in DMEM/F17 with/without serum, the medium is replaced with fresh medium with or without serum. Three (3) days after transfection, a sample is taken from the culture medium of the 293 adherent cells. Subsequently cells are scraped, or suspension cells are pelleted, and transferred into a receptacle. For adhesion cells, after centrifugation to remove cellular pellet, a second sample is taken from the supernatant after scraping. Next, cell lysis is achieved by three consecutive freeze-thaw cycles (−80 C to 37 C) or adding detergent triton. Cellular debris is removed by centrifugation or depth filtration and sample 3 is taken from the medium. The samples are quantified for AAV particles by DNase resistant genome titration by DNA qPCR. The total production yield from such a transfection is equal to the particle concentration from sample 3.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278).

### Large-Scale Production

In some embodiments, AAV particle production may be modified to increase the scale of production. Large scale viral production methods according to the present disclosure may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Methods of increasing viral particle production scale typically comprise increasing the number of viral replication cells. In some embodiments, viral replication cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE® (Corning Corp., Corning, N.Y.) and NUNC™ CELL FACTORY™ (Thermo Scientific, Waltham, Mass.). In some cases, large-scale adherent cell surfaces may comprise from about 1,000 cm² to about 100,000 cm². In some cases, large-scale adherent cell cultures may comprise from about 10⁷ to about 10⁹ cells, from about 10⁸ to about 10¹⁰ cells, from about 10⁹ to about 10¹² cells or at least 10¹² cells. In some cases, large-scale adherent cultures may produce from about 10⁹ to about 10¹², from about 101¹⁰ to about 10¹³, from about 10¹¹ to about 10¹⁴, from about 10¹² to about 10¹⁵ or at least 10¹⁵ viral particles.

In some embodiments, large-scale viral production methods of the present disclosure may comprise the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate), organic compounds [e.g. polyethyleneimine (PEI)] or the use of non-chemical methods (e.g. electroporation.) With cells grown in suspension, transfection methods may include, but are not limited to the use of calcium phosphate and the use of PEI. In some cases, transfection of large scale suspension cultures may be carried out according to the section entitled “Transfection Procedure” described in Feng, L. et al., 2008. Biotechnol Appl. Biochem. 50:121-32, the contents of which are herein incorporated by reference in their entirety. According to such embodiments, PEI-DNA complexes may be formed for introduction of plasmids to be transfected. In some cases, cells being transfected with PEI-DNA complexes may be ‘shocked’ prior to transfection. This comprises lowering cell culture temperatures to 4° C. for a period of about 1 hour. In some cases, cell cultures may be shocked for a period of from about 10 minutes to about 5 hours. In some cases, cell cultures may be shocked at a temperature of from about 0° C. to about 20° C.

In some cases, transfections may include one or more vectors for expression of an RNA effector molecule to reduce expression of nucleic acids from one or more AAV payload constructs. Such methods may enhance the production of viral particles by reducing cellular resources wasted on expressing payload constructs. In some cases, such methods may be carried out according to those methods taught in US Publication No. US2014/0099666, the contents of which are herein incorporated by reference in their entirety.

### III. Methods and Uses of the Compositions of the Disclosure

The AAV particles of the present disclosure, having enhanced tropism for a desired target tissue, may be used for the delivery of a payload to the target tissue. In certain embodiments the payload is a polypeptide. In a second embodiment, the payload is an antibody. In a third embodiment, the payload is an RNAi agent and/or modulatory polynucleotide. The AAV particles of the present disclosure may be delivered by intravenous injection or infusion for the targeting of CNS and/or PNS tissues (e.g., neurons, DRG).

The AAV particles of the present disclosure may be used for regulating expression of a gene of interest (or its protein product) in a cell, tissue, organ or subject.

In accordance with the present disclosure, methods for increasing expression of a target protein in a cell, tissue, organ or subject are provided; the method comprising administering to the cell, tissue, organ or subject an effective amount of the AAV particles of the disclosure comprising a viral genome with a payload region encoding the target protein.

Accordingly, the target protein may be increased by at least about 10%, preferably by at least about 10%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In certain embodiments, the AAV particles of the present disclosure may be used to increase the expression of a target protein in a cell of the CNS, such as a neuron, astrocyte, microglial cell, and/or oligodendrocyte. In some embodiments, the gene may encode a protein including, but not limited to, an antibody, aromatic L-amino acid decarboxylase (AADC), survival motor neuron 1 (SMN1), frataxin (FXN), APOE (APOE2, APOE3, or APOE4), GBA1, GRN, ASPA, CLN2, GLB1, SGSH, NAGLU, IDS, NPC1, or GAN.

In accordance with the present disclosure, methods for decreasing, inhibiting or suppressing the expression of a target gene or protein in a cell, tissue, organ or subject are also provided; the method comprising administering to the cell, tissue, organ or subject an effective amount of the AAV particles of the disclosure comprising a viral genome with a payload region encoding an RNAi agent and/or modulatory polynucleotide.

Accordingly, the RNAi agent may be, but is not limited to, dsRNA, siRNA, shRNA, pre-miRNA, pri-miRNA, miRNA, stRNA, lncRNA, piRNA, or snoRNA specific for a target gene of interest. Non-limiting examples of a target gene of interest include, SOD1, MAPT, APOE, HTT, C9ORF72, TDP-43, APP, BACE, SNCA, ATXN1, ATXN2, ATXN3, ATXN7, SCN1A-SCN5A, or SCN8A-SCN11A.

In some embodiments, the present disclosure provides methods for inhibiting/silencing target gene expression in a cell. Accordingly, the RNAi agent can be used to substantially inhibit target gene expression in a cell, such as but not limited to, in astrocytes, microglia, or cortical, hippocampal, entorhinal, thalamic, motor or sensory neurons. In some aspects, the inhibition of target gene expression refers to an inhibition by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the gene to be inhibited may include but is not limited to SOD1, MAPT, APOE, HTT, C9ORF72, TDP43, APP, BACE, SNCA, ATXN1, ATXN2, ATXN3, ATXN7, SCN1A-SCN5A, or SCN8A-SCN11A.

### Therapeutic Applications

The present disclosure provides a method for treating a disease, disorder and/or condition in a mammalian subject, including a human subject, comprising administering to the subject any of the AAV particles described herein or administering to the subject any of the described compositions, including pharmaceutical compositions, described herein.

In certain embodiments, the AAV particles of the present disclosure are administered to a subject prophylactically, to prevent on-set of disease. In another embodiment, the AAV particles of the present disclosure are administered to treat (lessen the effects of) a disease or symptoms thereof. In yet another embodiment, the AAV particles of the present disclosure are administered to cure (eliminate) a disease. In another embodiment, the AAV particles of the present disclosure are administered to prevent or slow progression of disease. In yet another embodiment, the AAV particles of the present disclosure are used to reverse the deleterious effects of a disease. Disease status and/or progression may be determined or monitored by standard methods known in the art.

In some embodiments, the AAV particles of the disclosure are useful in the field of medicine for the treatment, prophylaxis, palliation or amelioration of neurological diseases and/or disorders.

In some embodiments, the AAV particles of the disclosure are useful in the field of medicine for the treatment, prophylaxis, palliation or amelioration of tauopathy.

In some embodiments, the AAV particles of the disclosure are useful in the field of medicine for the treatment, prophylaxis, palliation or amelioration of Alzheimer's Disease.

In some embodiments, the AAV particles of the disclosure are useful in the field of medicine for the treatment, prophylaxis, palliation or amelioration of Friedreich's ataxia, or any disease stemming from a loss or partial loss of frataxin protein.

In some embodiments, the AAV particles of the disclosure are useful in the field of medicine for the treatment, prophylaxis, palliation or amelioration of Parkinson's Disease.

In some embodiments, the AAV particles of the disclosure are useful in the field of medicine for the treatment, prophylaxis, palliation or amelioration of Amyotrophic lateral sclerosis.

In some embodiments, the AAV particles of the disclosure are useful in the field of medicine for the treatment, prophylaxis, palliation or amelioration of Huntington's Disease.

In some embodiments, the AAV particles of the disclosure are useful in the field of medicine for the treatment, prophylaxis, palliation or amelioration of chronic or neuropathic pain.

In some embodiments, the AAV particles of the disclosure are useful in the field of medicine for treatment, prophylaxis, palliation or amelioration of a disease associated with the central nervous system.

In some embodiments, the AAV particles of the disclosure are useful in the field of medicine for treatment, prophylaxis, palliation or amelioration of a disease associated with the peripheral nervous system.

In certain embodiments, the AAV particles of the present disclosure are administered to a subject having at least one of the diseases or symptoms described herein.

As used herein, any disease associated with the central or peripheral nervous system and components thereof (e.g., neurons) may be considered a “neurological disease”.

Any neurological disease may be treated with the AAV particles of the disclosure, or pharmaceutical compositions thereof, including but not limited to, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimcer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Amold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavemomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Chareot-Marie-Tooth Disease, Chiari Malformation, Cholesterol Ester Storage Disease, Chorea, Choreoacanthocytosis, Chronic Inflammatory Demyclinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Balo's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloccles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritifonmis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsboume syndrome, Kleine-Levin Syndrome, Klippel-Feil Syndrome, Klippel-Trenaunay Syndrome (KTS), Kluver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamova Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy—Congenital, Myopathy—Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromvotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavemosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myclopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorca, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

### Methods of Treatment of Neurological Disease

AAV Particles Encoding protein payloads

Provided in the present disclosure are methods for introducing the AAV particles of the present disclosure into cells, the method comprising introducing into said cells any of the vectors in an amount sufficient for an increase in the production of target mRNA and protein to occur. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes.

Disclosed herein are methods for treating neurological disease associated with insufficient function/presence of a target protein (e.g., ApoE, FXN) in a subject in need of treatment. The method optionally comprises administering to the subject a therapeutically effective amount of a composition comprising AAV particles of the present disclosure. As a non-limiting example, the AAV particles can increase target gene expression, increase target protein production, and thus reduce one or more symptoms of neurological disease in the subject such that the subject is therapeutically treated.

In certain embodiments, the composition comprising the AAV particles of the present disclosure is administered to the central nervous system of the subject via systemic administration. In certain embodiments, the systemic administration is intravenous injection.

In some embodiments, the composition comprising the AAV particles of the present disclosure is administered to the central nervous system of the subject. In other embodiments, the composition comprising the AAV particles of the present disclosure is administered to a CNS tissue of a subject (e.g., putamen, thalamus or cortex of the subject).

In certain embodiments, the composition comprising the AAV particles of the present disclosure is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In certain embodiments, the composition comprising the AAV particles of the present disclosure is administered to the central nervous system of the subject via intraparenchymal injection and intravenous injection.

In certain embodiments, the AAV particles of the present disclosure may be delivered into specific types of targeted cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells.

In certain embodiments, the AAV particles of the present disclosure may be delivered to neurons in the putamen, thalamus and/or cortex.

In some embodiments, the AAV particles of the present disclosure may be used as a therapy for neurological disease.

In some embodiments, the AAV particles of the present disclosure may be used as a therapy for tauopathies.

In some embodiments, the AAV particles of the present disclosure may be used as a therapy for Alzheimer's Disease.

In some embodiments, the AAV particles of the present disclosure may be used as a therapy for Amyotrophic Lateral Sclerosis.

In some embodiments, the AAV particles of the present disclosure may be used as a therapy for Huntington's Disease.

In some embodiments, the AAV particles of the present disclosure may be used as a therapy for Parkinson's Disease.

In some embodiments, the AAV particles of the present disclosure may be used as a therapy for Friedreich's Ataxia.

In some embodiments, the AAV particles of the present disclosure may be used as a therapy for chronic or neuropathic pain.

In certain embodiments, administration of the AAV particles described herein to a subject may increase target protein levels in a subject. The target protein levels may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may increase the protein levels of a target protein by at least 50%. As a non-limiting example, the AAV particles may increase the proteins levels of a target protein by at least 40%. As a non-limiting example, a subject may have an increase of 10% of target protein. As anon-limiting example, the AAV particles may increase the protein levels of a target protein by fold increases over baseline. In certain embodiments, AAV particles lead to 5-6 times higher levels of a target protein.

In certain embodiments, administration of the AAV particles described herein to a subject may increase the expression of a target protein in a subject. The expression of the target protein may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may increase the expression of a target protein by at least 50%. As a non-limiting example, the AAV particles may increase the expression of a target protein by at least 40%.

In certain embodiments, intravenous administration of the AAV particles described herein to a subject may increase the CNS expression of a target protein in a subject. The expression of the target protein may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may increase the expression of a target protein in the CNS by at least 50%. As a non-limiting example, the AAV particles may increase the expression of a target protein in the CNS by at least 40%.

In some embodiments, the AAV particles of the present disclosure may be used to increase target protein expression in astrocytes in order to treat a neurological disease. Target protein in astrocytes may be increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-9500, 80-90%, 80-95%, or 90-95%.

In some embodiments, the AAV particles may be used to increase target protein in microglia. The increase of target protein in microglia may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55% 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-.50%, 20-.55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-7500, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the AAV particles may be used to increase target protein in cortical neurons. The increase of target protein in the cortical neurons may be, independently, increased by, 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60% 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the AAV particles may be used to increase target protein in hippocampal neurons. The increase of target protein in the hippocampal neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the AAV particles may be used to increase target protein in DRG and/or sympathetic neurons. The increase of target protein in the DRG and/or sympathetic neurons may be, independently, increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the AAV particles of the present disclosure may be used to increase target protein in sensory neurons in order to treat neurological disease. Target protein in sensory neurons may be increased by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In some embodiments, the AAV particles of the present disclosure may be used to increase target protein and reduce symptoms of neurological disease in a subject. The increase of target protein and/or the reduction of symptoms of neurological disease may be, independently, altered (increased for the production of target protein and reduced for the symptoms of neurological disease) by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-255%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, the AAV particles of the present disclosure may be used to reduce the decline of functional capacity and activities of daily living as measured by a standard evaluation system such as, but not limited to, the total functional capacity (TFC) scale.

In certain embodiments, the AAV particles of the present disclosure may be used to improve performance on any assessment used to measure symptoms of neurological disease. Such assessments include, but are not limited to ADAS-cog (Alzheimer Disease Assessment Scale—cognitive), MMSE (Mini-Mental State Examination), GDS (Geriatric Depression Scale), FAQ (Functional Activities Questionnaire). ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment). ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (informant Questionnaire on Cognitive Decline in the Elderly), Neuropsychiatric Inventory. The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

In some embodiments, the present composition is administered as a solo therapeutic or as combination therapeutic for the treatment of neurological disease.

The AAV particles encoding the target protein may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the AAV particles of the present disclosure can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation. As a non-limiting example, the combination therapy may be in combination with one or more neuroprotective agents such as small molecule compounds, growth factors and hormones which have been tested for their neuroprotective effect on motor neuron degeneration.

Compounds tested for treating neurological disease which may be used in combination with the AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetycholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chora and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles of the present disclosure for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particle described herein may be co-administered with AAV particles expressing neurotrophic factors such as AAV-IGF-I (See e.g., Vincent et al., Neuromolecular medicine, 2004, 6, 79-85; the contents of which are incorporated herein by reference in their entirety) and AAV-GDNF (See e.g., Wang et al., J Neurosci., 2002, 22, 6920-6928; the contents of which are incorporated herein by reference in their entirety).

In certain embodiments, administration of the AAV particles to a subject will increase the expression of a target protein in a subject and the increase of the expression of the target protein will reduce the effects and/or symptoms of neurological disease in a subject.

As a non-limiting example, the target protein may be an antibody, or fragment thereof.

AAV Particles Comprising RNAi agents or Modulatory Polynucleotides

Provided in the present disclosure are methods for introducing the AAV particles of the disclosure, comprising a viral genome with a nucleic acid sequence encoding one or more siRNA molecules into cells, the method comprising introducing into said cells any of the vectors in an amount sufficient for degradation of a target mRNA to occur, thereby activating target-specific RNAi in the cells. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes.

Disclosed herein are methods for treating neurological diseases associated with dysfunction of a target protein in a subject in need of treatment. The method optionally comprises administering to the subject a therapeutically effective amount of a composition comprising AAV particles comprising a viral genome with a nucleic acid sequence encoding one or more siRNA molecules. As a non-limiting example, the siRNA molecules can silence target gene expression, inhibit target protein production, and reduce one or more symptoms of neurological disease in the subject such that the subject is therapeutically treated.

In some embodiments, the composition comprising the AAV particles of the present disclosure comprising a viral genome encoding one or more siRNA molecules comprise an AAV capsid that allows for enhanced transduction of CNS and/or PNS cells after intravenous administration.

In some embodiments, the composition comprising the AAV particles of the present disclosure with a viral genome encoding at least one siRNA molecule is administered to the central nervous system of the subject. In other embodiments, the composition comprising the AAV particles of the present disclosure is administered to a tissue of a subject (e.g., putamen, thalamus or cortex of the subject).

In certain embodiments, the composition comprising the AAV particles of the disclosure, comprising a viral genome with a nucleic acid sequence encoding one or more siRNA molecules is administered to the central nervous system of the subject via systemic administration. In certain embodiments, the systemic administration is intravenous injection.

In certain embodiments, the composition comprising the AAV particles of the disclosure comprising a viral genome with a nucleic acid sequence encoding one or more siRNA molecules is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In certain embodiments, the composition comprising the AAV particles comprising a viral genome with a nucleic acid sequence encoding one or more siRNA molecules is administered to the central nervous system of the subject via intraparenchymal injection and intravenous injection.

In certain embodiments, the AAV particles comprising a viral genome with a nucleic acid sequence encoding one or more siRNA molecules may be delivered into specific types or targeted cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells.

In certain embodiments, the AAV particles comprising a viral genome with a nucleic acid sequence encoding one or more siRNA molecules may be delivered to neurons in the putamen, thalamus, and/or cortex.

In certain embodiments, the AAV particles comprising a viral genome with a nucleic acid sequence encoding one or more siRNA molecules may be used as a therapy for neurological disease.

In certain embodiments, the AAV particles comprising a viral genome with a nucleic acid sequence encoding one or more siRNA molecules may be used as a therapy for tauopathies.

In certain embodiments, the AAV particles comprising a viral genome with a nucleic acid sequence encoding one or more siRNA molecules may be used as a therapy for Alzheimer's Disease.

In certain embodiments, the AAV particles comprising a viral genome with a nucleic acid sequence encoding one or more siRNA molecules may be used as a therapy for Amyotrophic Lateral Sclerosis.

In certain embodiments, the AAV particles comprising a viral genome with a nucleic acid sequence encoding one or more siRNA molecules may be used as a therapy for Huntington's Disease.

In certain embodiments, the AAV particles comprising a viral genome with a nucleic acid sequence encoding one or more siRNA molecules may be used as a therapy for Parkinson's Disease.

In certain embodiments, the AAV particles comprising a viral genome with a nucleic acid sequence encoding one or more siRNA molecules may be used as a therapy for Friedreich's Ataxia.

In certain embodiments, the administration of AAV particles comprising a viral genome with a nucleic acid sequence encoding one or more siRNA molecules to a subject may lower target protein levels in a subject. The target protein levels may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may lower the protein levels of a target protein by at least 50%. As a non-limiting example, the AAV particles may lower the protein levels of a target protein by at least 40%.

In certain embodiments, the administration of AAV particles comprising a viral genome with a nucleic acid sequence encoding one or more siRNA molecules to a subject may lower the expression of a target protein in a subject. The expression of a target protein may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-950%, 90-100% or 95-100% in a subject such as, but not limited to, the CNS, a region of the CNS, or a specific cell of the CNS of a subject. As a non-limiting example, the AAV particles may lower the expression of a target protein by at least 50%. As a non-limiting example, the AAV particles may lower the expression of a target protein by at least 40%.

In certain embodiments, the administration of AAV particles comprising a viral genome with a nucleic acid sequence encoding one or more siRNA molecules to a subject may lower the expression of a target protein in the CNS of a subject. The expression of a target protein may be lowered by about 300%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting example, the AAV particles may lower the expression of a target protein by at least 50%. As a non-limiting example, the AAV particles may lower expression of a target protein by at least 40%.

In certain embodiments, the AAV particles comprising a viral genome with a nucleic acid sequence encoding one or more siRNA molecules may be used to suppress a target protein in astrocytes in order to treat neurological disease. Target protein in astrocytes may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 75-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. Target protein in astrocytes may be reduced by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 70-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, the AAV particles comprising a viral genome with a nucleic acid sequence encoding one or more siRNA molecules may be used to suppress a target protein in microglia. The suppression of the target protein in microglia may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, the AAV particles comprising a viral genome with a nucleic acid sequence encoding one or more siRNA molecules may be used to suppress target protein in cortical neurons. The suppression of a target protein in cortical neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, the AAV particles comprising a viral genome with a nucleic acid sequence encoding one or more siRNA molecules may be used to suppress a target protein in hippocampal neurons. The suppression of a target protein in the hippocampal neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80% 10-85% 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55% 40-60% 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, the AAV particles comprising a viral genome with a nucleic acid sequence encoding one or more siRNA molecules may be used to suppress a target protein in DRG and/or sympathetic neurons. The suppression of a target protein in the DRG and/or sympathetic neurons may be, independently, suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45% 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. The reduction may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, the AAV particles comprising a viral genome with a nucleic acid sequence encoding one or more siRNA molecules may be used to suppress a target protein in sensory neurons in order to treat neurological disease. Target protein in sensory neurons may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%0, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%. Target protein in the sensory neurons may be reduced by 5%, 10%, 15%, 20%, 25%, 30%, 35%, 40%, 45% 50%, 55%, 60% 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, the AAV particles comprising a viral genome with a nucleic acid sequence encoding one or more siRNA molecules may be used to suppress a target protein and reduce symptoms of neurological disease in a subject. The suppression of target protein and/or the reduction of symptoms of neurological disease may be, independently, reduced or suppressed by 5%, 10%, 15%, 20%, 25%, 30%, 35%, 40%, 45%, 50%, 55%, 60%, 65%, 70%, 75%, 80%, 85% 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 0.5-90%, 0.5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, the AAV particles comprising a viral genome with a nucleic acid sequence encoding one or more siRNA molecules may be used to reduce the decline of functional capacity and activities of daily living as measured by a standard evaluation system such as, but not limited to, the total functional capacity (TFC) scale.

In some embodiments, the present composition is administered as a solo therapeutic or as combination therapeutic for the treatment of neurological disease.

The AAV particles comprising a viral genome with a nucleic acid sequence encoding one or more siRNA molecules may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the AAV particles comprising a viral genome with a nucleic acid sequence encoding one or more siRNA molecules can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation.

Compounds tested for treating neurological disease which may be used in combination with the AAV particles comprising a viral genome with a nucleic acid sequence encoding one or more siRNA molecules include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK30 (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetycholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chora and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles comprising a viral genome with a nucleic acid sequence encoding one or more siRNA molecules for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particle encoding the nucleic acid sequence for the at least one siRNA duplex targeting the gene of interest may be co-administered with AAV particles expressing neurotrophic factors such as AAV-IGF-I (See e.g., Vincent et al., Neuromolecular medicine, 2004, 6, 79-85; the content of which is incorporated herein by reference in its entirety) and AAV-GDNF (See e.g., Wang et al., J Neurosci., 2002, 22, 6920-6928; the contents of which are incorporated herein by reference in their entirety).

In certain embodiments, administration of the AAV particles to a subject will reduce the expression of a target protein in a subject and the reduction of expression of the target protein will reduce the effects and/or symptoms of neurological disease in a subject.

### IV. Formulation

### Pharmaceutical Compositions

According to the present disclosure the AAV particles may be prepared as pharmaceutical compositions. As used herein the term “pharmaceutical composition” refers to compositions comprising at least one active ingredient and optionally, one or more pharmaceutically acceptable excipients.

Relative amounts of the active ingredient (e.g. AAV particle), a pharmaceutically acceptable excipient, and/or any additional ingredients in a pharmaceutical composition in accordance with the present disclosure may vary. Differences in the constitution of a pharmaceutical composition may depend upon the identity, size, and/or condition of the subject being treated, the route by which the composition is to be administered, the nature of the AAV particle composition or the excipient, and/or any other factor. The composition may comprise between 0.0001% and 99% (w/w) of the active ingredient. By way of example, the composition may comprise between 0.00010% and 100%, e.g., between 0.5 and 50%, between 1-30%, between 5-80%, or at least 80% (w/w) active ingredient.

In certain embodiments, the pharmaceutical composition comprises AAV particles having one type of AAV capsid protein. In another embodiment, the pharmaceutical composition comprises a mixture of AAV particles, having different AAV capsid proteins.

In some embodiments, the pharmaceutical compositions described herein may comprise AAV particles comprising a viral genome encoding at least one payload. As a non-limiting example, the pharmaceutical compositions may contain an AAV particle with 1, 2, 3, 4, 5, or more payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, pharmaceutical compositions are administered to humans, human patients or subjects.

In certain embodiments, the pharmaceutical composition is administered to the human, human patient, and/or subject by intravenous delivery. The pharmaceutical composition may be prepared in a manner optimized for administration by intravenous delivery.

### Formulation

Formulations of the present disclosure can include, without limitation, one or more AAV particles, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology.

In general, such preparatory methods include the step of associating the active ingredient with an excipient and/or one or more other accessory ingredients. As used herein, the phrase “active ingredient” generally refers either to an AAV particle with a viral genome encoding a payload or to the end product, or payload itself.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, the AAV formulations described herein may contain sufficient AAV particles for expression of at least one expressed functional payload. As a non-limiting example, the AAV particles may contain viral genomes encoding 1, 2, 3, 4 or 5 functional payloads.

In some embodiments, the formulations described herein may contain at least one AAV particle comprising a viral genome with a nucleic acid sequence encoding a protein of interest. The protein of interest may include but is not limited to an antibody, aromatic L-amino acid decarboxylase (AADC), survival motor neuron 1 (SMN1), frataxin (FXN), APOE (APOE2, APOE3, or APOE4), GBA1, GRN, ASPA, CLN2, GLB1, SGSH, NAGLU, IDS, NPC1, or GAN.

In some embodiments, the formulations described herein may contain at least one AAV particle comprising a viral genome with a nucleic acid sequence encoding the siRNA molecules described herein. In certain embodiments, the siRNA molecules may target a gene of interest at one target site. In another embodiment, the formulation comprises a plurality of AAV particles, each AAV particle comprising a viral genome with a nucleic acid sequence encoding a siRNA molecule targeting the gene of interest at a different target site. The target gene may be targeted at 1, 2, 3, 4, 5 or more than 5 sites. In certain embodiments, the target gene may include, but is not limited to, SOD1, MAPT, APOE, HTT, C9ORF72, TDP-43, APP. BACE, SNCA, ATXN1, ATXN2, ATXN3, ATXN7, SCN1A-SCN5A, or SCN8A-SCN11A.

According to the present disclosure AAV particles may be formulated for systemic delivery. In another embodiment. AAV particles may be formulated for CNS delivery. The formulation of AAV particles may be optimized for delivery to any target tissue by any administration route described herein. As a non-limiting example, the formulation of AAV particles may be optimized for CNS as the target tissue and intravenous administration as the method of delivery. Alternatively, the formulation of AAV particles may be optimized for DRG as the target tissue and intravenous administration as the method of delivery.

The AAV particles of the disclosure can be formulated using one or more excipients to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott. Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, AAV particle formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA). Inactive ingredients and their use are well known in the art.

Pharmaceutical composition formulations of AAV particles disclosed herein may include cations or anions. In certain embodiments, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations of the disclosure may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

In certain embodiments, the AAV particles of the disclosure may be formulated in phosphate buffered saline (PBS), in combination with an ethylene oxide/propylene oxide copolymer (also known as pluronic or poloxamer).

In certain embodiments, the AAV particles of the disclosure may be formulated in PBS with 0.001% pluronic acid (F-68) (poloxamer 188) at a pH of about 7.0.

In certain embodiments, the AAV particles of the disclosure may be formulated in PBS with 0.001% pluronic acid (F-68) (poloxamer 188) at a pH of about 7.3.

In certain embodiments, the AAV particles of the disclosure may be formulated in PBS with 0.001% pluronic acid (F-68) (poloxamer 188) at a pH of about 7.4.

In certain embodiments, the AAV particles of the disclosure may be formulated in a solution comprising sodium chloride, sodium phosphate and an ethylene oxide/propylene oxide copolymer.

In certain embodiments, the AAV particles of the disclosure may be formulated in a solution comprising sodium chloride, sodium phosphate dibasic, sodium phosphate monobasic and poloxamer 188/pluronic acid (F-68).

In certain embodiments, the AAV particles of the disclosure may be formulated in a solution comprising sodium chloride, potassium phosphate monobasic, sodium phosphate dibasic, and poloxamer 188/pluronic acid (F-68).

In certain embodiments, AAV particles are formulated in a solution comprising about 200 mM sodium chloride (NaCl), about 1 mM potassium phosphate monobasic (KH₂PO₄), about 3 mM sodium phosphate dibasic (Na₂HPO₄), and about 0.001% Pluronic F-68/poloxamer 188 at a pH of about 7.4. The concentration of sodium chloride in the final solution may be 150 mM-250 mM. As non-limiting examples, the concentration of sodium chloride in the final solution may be 150 mM, 160 mM, 170 mM, 180 mM, 190 mM, 200 mM, 210 mM, 220 mM, 230 mM, 240 mM, 250 mM, or any concentration in between. The concentration of potassium phosphate monobasic in the final solution may be 0.01 mM-3 mM. As non-limiting examples, the concentration of potassium phosphate monobasic in the final solution may be 0.01 mM, 0.5 mM, 1 mM, 2 mM, 3 mM, or any concentration in between. The concentration of sodium phosphate dibasic in the final solution may be 1 mM-10 mM. As non-limiting examples, the concentration of sodium phosphate dibasic in the final solution may be 1 mM, 2 mM, 3 mM, 4 mM, 5 mM, 6 mM, 7 mM, 8 mM, 9 mM, 10 mM or any concentration in between. The concentration of pluronic F-68 (poloxamer 188) may be 0.0001%-1%. As non-limiting examples, the concentration of pluronic F-68 (poloxamer 188) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

### V. Dosing and Administration

In certain embodiments, the AAV particles may be administered to a subject in a therapeutically effective amount to reduce the symptoms of disease, such as a neurological disease (affecting either the CNS or PNS), of a subject (e.g., determined using a known evaluation method). In some embodiments the subject is a mammal. A mammal may include, a mouse, a non-human primate, and/or a human.

### Administration

The AAV particles of the present disclosure may be administered by any delivery route which results in a therapeutically effective outcome. These include, but are not limited to, intravenous (into a vein), intraparenchymal (into the substance of e.g., CNS), intraparenchymal (brain), intraparenchymal (spinal cord), intraparenchymal (DRG), intracranial, intrastriatal, intrathalamic, enteral (into the intestine), gastroenteral, epidural (into the dura mater), oral (by way of the mouth), transdermal, intracerebral (into the cerebrum), intracerebroventricular (into the cerebral ventricles), sub-pial (between pia and CNS parenchyma), intracarotid arterial (into the intracarotid artery), epicutaneous (application onto the skin), intradermal, (into the skin itself), subcutaneous (under the skin), nasal administration (through the nose), intravenous bolus, intravenous drip, intra-arterial (into an artery), systemic, intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracistemal (within the cisterna magna cerebellomedularis), intracomeal (within the comea), dental intracoronal, intracoronary (within the coronary arteries), intracorporus cavemosum (within the dilatable spaces of the corporus cavemosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), larvngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

In certain embodiments, the AAV particles of the disclosure are administered by intraparenchymal injection. In other words, the AAV particles are delivered directly to the target tissue. AAV particles of the disclosure may be administered by intraparenchymal delivery to central nervous system tissue, such as but not limited to, the putamen, the thalamus, and/or the cortex. AAV particles of the disclosure may be administered by intraparenchymal delivery to the peripheral nervous system tissue, such as, but not limited to, the dorsal root ganglia.

In some embodiments, the AAV particles that may be administered to a subject by peripheral injections. AAV particles may be administered systemically. In certain embodiments, AAV particles are administered intravenously.

Intravenous administration encompasses the use of any vein of the subject for the delivery of the AAV particles of the disclosure. Non-limiting examples of target veins include, median cubital vein, orbital veins (superior or inferior ophthalmic, saphenous veins (greater or lesser), internal jugular, and/or femoral vein. When the subject is a mouse, the target vein may be the tail vein or the orbital veins.

In certain embodiments, the AAV particles of the present disclosure may be administered via intravenous delivery to cells of the central nervous system (CNS), such as, but not limited to, neurons, astrocytes, microglia and/or oligodendrocytes.

In certain embodiments, the AAV particles of the present disclosure may be administered via intravenous delivery to cells of the peripheral nervous system (PNS), such as, but not limited to, neurons and/or Schwann cells.

In certain embodiments, the AAV particles of the present disclosure may be administered via intravenous delivery to DRG neurons.

In certain embodiments, the AAV particles may be delivered by injection into a CSF pathway (e.g., intrathecal or intraventricular).

In certain embodiments, the AAV particles of the present disclosure may be administered to a subject by intracranial delivery (See, e.g., U.S. Pat. No. 8,119,611; the content of which are incorporated herein by reference in their entirety).

In certain embodiments, the AAV particle may be administered to the CNS or PNS in a therapeutically effective amount to improve function and/or survival for a subject with a neurological disease. As a non-limiting example, the vector may be administered intravenously.

The AAV particles of the present disclosure may be administered in any suitable form, either as a liquid solution or suspension, as a solid form suitable for liquid solution or suspension in a liquid solution. The AAV particles may be formulated with any appropriate and pharmaceutically acceptable excipient.

In some embodiments, the AAV particles of the present disclosure may be delivered to a subject via a single route of administration. In other embodiments, the AAV particles may be delivered to a subject via more than one route of administration.

In some embodiments, the AAV particles of the present disclosure may be delivered to a subject via a multi-site route of administration. AAV particles may be administered at 2, 3, 4, 5 or more than 5 sites.

In some embodiments, two or more compositions comprising AAV particles may be administered to the same subject. In some embodiments, the two or more AAV particle compositions may be administered via the same route. In some embodiments, the two or more AAV particle compositions may be administered via different routes. Two or more compositions comprising AAV particles may be administered simultaneously, or at different times. In some embodiments, one composition comprising AAV particles may be administered by an intraparenchymal route, while another composition comprising AAV particles may be administered by an intravenous route.

In some embodiments. AAV particles may be administered by injection. In some embodiments, AAV particles are administered by infusion. In some embodiments, AAVs may be administered as a bolus.

Injection sites for AAV particle administration may include, but are not limited to, the putamen, thalamus, cortex, hippocampus, dorsal root ganglia, and deep cerebellar nuclei.

In certain embodiments, administration of the AAV particles of the present disclosure occurs only once, and serves as a one-time treatment. In other embodiments, administration of the AAV particles of the present disclosure occurs more than once.

### Dosing

The present disclosure provides methods of administering AAV particles in accordance with the disclosure to a subject in need thereof. The pharmaceutical, diagnostic, or prophylactic AAV particles and compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal.

Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific AAV particle employed; the duration of the treatment; drugs used in combination or coincidental with the specific AAV particle employed; and like factors well known in the medical arts.

In certain embodiments. AAV particle pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight to obtain the desired therapeutic, diagnostic, or prophylactic effect. It will be understood that the above dosing concentrations may be converted to vg or viral genomes per kg or into total viral genomes administered by one of skill in the art.

In certain embodiments, AAV particle pharmaceutical compositions in accordance with the present disclosure may be administered at about 0.1-600 μl/site, or about 0.1 to about 0.5 μl/site, about 0.5 to about 1 μl/site, about 1 to about 10 μl/site, about 10 to about 600 μl/site, about 50 to about 500 μl/site, about 100 to about 400 μl/site, about 120 to about 300 μl/site, about 140 to about 200 μl/site, about 160 μl/site. As non-limiting examples, AAV particles may be administered at 0.5 μl/site, 50 μl/site, 150 μl/site, 160 μl/site or 250 μl/site.

In certain embodiments, delivery of the compositions in accordance with the present disclosure to cells comprises a rate of delivery defined by [VG/hour=mL/hour*VG/mL] wherein VG is viral genomes, VG/mL is composition concentration, and mL/hour is rate of delivery.

In certain embodiments, delivery of AAV particle compositions to cells may comprise a total concentration per subject between about 1×10⁶ VG (viral genome) and about 1×10⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷. 6×10⁷, 7×10⁷, 8×10⁷, 9×10⁷, 1×10⁸. 2×10⁸, 3×10⁸, 4×10⁸, 5×10⁸, 6×10⁸. 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 4×10¹⁰, 5×10¹⁰, 6×10¹⁰, 7×10¹⁰, 7.5×10¹⁰, 8×10¹⁰, 9×10¹⁰, 1×10¹¹, 1.25×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.75×10¹¹, 4×10¹¹, 5×10¹¹, 6×10¹¹, 6.25×10¹¹, 6.75×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10², 1.2×10², 1.3×10¹², 1.4×10¹², 1.5×10¹², 1.6×10¹², 1.7×10¹², 1.8×10¹², 1.9×10¹², 2×10¹², 3×1⁰². 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴. 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject.

In certain embodiments, delivery of AAV particle compositions to cells may comprise a total concentration per subject between about 1×10⁶ VG/kg and about 1×10¹⁶ VG/kg. In some embodiments, delivery may comprise a composition concentration of about 1×10⁶, 2×10⁶, 3×10⁶, 4×10⁶. 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10², 8.2×10¹², 8.3×10¹². 8.4×10¹², 8.5×10¹², 8.6×10¹², 8.7×10¹², 8.8×10¹², 8.9×10¹², 9×10¹², 1×10¹³. 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/kg. In certain embodiments, the delivery comprises a composition concentration of 1×10¹³ VG/kg. In certain embodiments, the delivery comprises a composition concentration of 2.1×10¹² VG/kg. In certain embodiments, the delivery comprises a composition concentration of 1×10¹³ VG/kg. In certain embodiments, the delivery comprises a composition concentration of 6.7×10¹² VG/kg. In certain embodiments, the delivery comprises a composition concentration of 7×10¹² VG/kg. In certain embodiments, the delivery comprises a composition concentration of 2×10¹³ VG/kg. In certain embodiments, the delivery comprises a composition concentration of 3×10¹¹ VG/kg. In certain embodiments, the delivery comprises a composition concentration of 3×10¹² VG/kg. In certain embodiments, the delivery comprises a composition concentration of 3×10¹³ VG/kg. In certain embodiments, the delivery comprises a composition concentration of 6.3×10¹² VG/kg.

In certain embodiments, delivery of AAV particle compositions to cells may comprise a total concentration per site between about 1×10⁶ VG/site and about 1×10¹⁶ VG/site. In some embodiments, delivery may comprise a composition concentration of about 1×10⁶, 2×10⁶. 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/site.

In certain embodiments, delivery of AAV particles to cells of the central nervous system may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, I×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 6.1×10¹², 6.2×10¹², 6.3×10¹², 6.4×10¹², 6.5×10¹², 6.6×10¹², 6.7×10¹². 6.8×10¹², 6.9×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As anon-limiting example, the total dose is 1×10¹³ VG. As another non-limiting example, the total dose is 2.1×10¹² VG. As another non-limiting example, the total dose is 6.3×10¹² VG.

In certain embodiments, about 10⁵ to 10⁶ viral genome (unit) may be administered per dose.

In certain embodiments, delivery of the compositions comprising the AAV particles in accordance with the present disclosure to cells may comprise a total concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×1¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹². 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹². 4.5×10¹², 4.6×10¹², 4.7×10¹², 4.8×10¹², 4.9×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL.

In certain embodiments, delivery of AAV particles to cells of the central nervous system may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷. 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 6.1×10¹², 6.2×10¹², 6.3×10¹², 6.4×10¹², 6.5×10¹², 6.6×10¹², 6.7×10¹², 6.8×10¹², 6.9×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In certain embodiments, the delivery comprises a composition concentration of 1×10¹³ VG/mL. In certain embodiments, the delivery comprises a composition concentration of 2.1×10¹² VG/mL. In certain embodiments, the delivery comprises a composition concentration of 1×10¹³ VG/mL. In certain embodiments, the delivery comprises a composition concentration of 2×10¹³ VG/mL. In certain embodiments, the delivery comprises a composition concentration of 3×10¹¹ VG/mL. In certain embodiments, the delivery comprises a composition concentration of 3×10¹² VG/mL. In certain embodiments, the delivery comprises a composition concentration of 6.3×10¹² VG/mL. In certain embodiments, the delivery comprises a composition concentration of 3×10³ VG/mL.

In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact. i.e., single administration event.

The desired dosage of the AAV particles of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24 hour period, may be administered by any of these methods, or as a combination of these methods, or by any other methods suitable for a pharmaceutical administration.

In certain embodiments, delivery of the AAV particles of the present disclosure to a subject provides regulating activity of a target gene in a subject. The regulating activity may be an increase in the production of the target protein in a subject or the decrease of the production of target protein in a subject. The regulating activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In some embodiments, the AAV particle of the present disclosure may be administered to a subject using a single dose, one-time treatment. The dose of the one-time treatment may be administered by any methods known in the art and/or described herein. As used herein, a “one-time treatment” refers to a composition which is only administered one time. If needed, a booster dose may be administered to the subject to ensure the appropriate efficacy is reached. A booster may be administered 1 month, 2 months, 3 months, 4 months, 5 months, 6 months, 7 months, 8 months, 9 months, 10 months, 11 months, 12 months, 1 year, 13 months, 14 months, 15 months, 16 months, 17 months, 18 months, 19 months, 20 months, 21 months, 22 months, 23 months, 24 months, 2 years, 3 years, 4 years, 5 years, 6 years, 7 years, 8 years, 9 years, 10 years, or more than 10 years after the one-time treatment.

### Delivery

In certain embodiments, the AAV particles or pharmaceutical compositions of the disclosure are delivered by intravenous injection.

In certain embodiments, the AAV particles or pharmaceutical compositions of the present disclosure may be administered or delivered using the methods for treatment of disease described in U.S. Pat. No. 8,999,948, or International Publication No. WO2014178863, the contents of each of which are herein incorporated by reference in their entirety.

In certain embodiments, the AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In certain embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of each of which are herein incorporated by reference in their entirety.

In certain embodiments, the AAV particle or pharmaceutical compositions of the present disclosure may be administered or delivered using the methods for delivering proteins using AAV vectors described in European Patent Application No. EP2678433, the contents of which are herein incorporated by reference in their entirety.

In certain embodiments, the AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In certain embodiments, the AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In certain embodiments, the AAV particle or pharmaceutical compositions of the present disclosure may be administered or delivered using the methods for delivering a payload described in U.S. Pat. No. 8,283,151, the contents of which are herein incorporated by reference in their entirety.

In certain embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In certain embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

### Delivery to Cells

The present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

The present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

In certain embodiments, the subject is a human. In some aspects, the human subject is a patient with a disease, for example, a neurological disease, or a disease associated with the central or peripheral nervous system or components thereof.

### Combinations

The AAV particles may be used in combination with one or more other therapeutic, prophylactic, research or diagnostic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### VI. Kits and Devices

### Kits

In certain embodiments, the disclosure provides a variety of kits for conveniently and/or effectively carrying out the methods described herein. Typically, kits will comprise sufficient amounts and/or numbers of components to allow a user to perform multiple treatments of a subject(s) and/or to perform multiple experiments.

Any of the targeting peptides and/or associated AAV particles of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In certain embodiments, the AAV particles of the disclosure are delivered by catheter.

In certain embodiments, the AAV particles of the disclosure are delivered by syringe and/or syringe pump.

In certain embodiments, the AAV particles of the disclosure are delivered by cannula.

In certain embodiments, the AAV particles of the disclosure are delivered by a device, wherein the device is further placed in a mechanism to direct or target the trajectory.

In certain embodiments, the AAV particles may be delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569, and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In certain embodiments, the AAV particles may be delivered using a method, system and/or computer program for positioning of an apparatus to a target point on a subject to deliver the AAV particles. As a non-limiting example, the method, system and/or computer program may be the methods, systems and/or computer programs described in U.S. Pat. No. 8,340,743, the contents of which are herein incorporated by reference in their entirety. The method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In certain embodiments, the AAV particles may be delivered to a subject using a convention-enhanced delivery device. Non-limiting examples of targeted delivery of drugs using convection are described in US Patent Publication Nos. US20100217228, US20130035574, and US 20130035660 and International Patent Publication No. WO2013019830 and WO2008144585, the contents of each of which are herein incorporated by reference in their entireties.

In certain embodiments, a subject may be imaged prior to, during and/or after delivery of the AAV particles. The imaging method may be a method known in the art and/or described herein, such as but not limited to, magnetic resonance imaging (MRI). As a non-limiting example, imaging may be used to assess therapeutic effect. As another non-limiting example, imaging may be used for assisted delivery of AAV particles.

In certain embodiments, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677, and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In certain embodiments, the AAV particles may be delivered using an MRI-compatible tip assembly. Non-limiting examples of MRI-compatible tip assemblies are described in US Patent Publication No. US20140275980, the contents of which is herein incorporated by reference in its entirety.

In certain embodiments, the AAV particles may be delivered using a cannula which is MRI-compatible. Non-limiting examples of MRI-compatible cannulas include those taught in International Patent Publication No. WO2011130107, the contents of which are herein incorporated by reference in its entirety.

In certain embodiments, the AAV particles may be delivered using a catheter which is MRI-compatible. Non-limiting examples of MRI-compatible catheters include those taught in International Patent Publication No. WO2012116265, U.S. Pat. No. 8,825,133 and US Patent Publication No. US20140024909, the contents of each of which are herein incorporated by reference in their entireties.

In certain embodiments, the AAV particles may be delivered using a device with an elongated tubular body and a diaphragm as described in US Patent Publication Nos. US20140276582 and US20140276614, the contents of each of which are herein incorporated by reference in their entireties.

In certain embodiments, the AAV particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In certain embodiments, the AAV particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In certain embodiments, the AAV particles may be delivered to a subject using a gene gun.

### VII. Definitions

Adeno-associated virus: As used herein, the term “adeno-associated virus” or “AAV” refers to members of the dependovirus genus comprising any particle, sequence, gene, protein, or component derived therefrom.

AAV Particle: As used herein, an “AAV particle” is a virus which comprises a capsid and a viral genome with at least one payload region and at least one ITR. As used herein “AAV particles of the disclosure” are AAV particles comprising a parent capsid sequence with at least one targeting peptide insert. AAV particles of the present disclosure may be produced recombinantly and may be based on adeno-associated virus (AAV) parent or reference sequences. AAV particles may be derived from any serotype, described herein or known in the art, including combinations of serotypes (i.e., “pseudotyped” AAV) or from various genomes (e.g., single stranded or self-complementary). In addition, the AAV particle may be replication defective and/or targeted. In certain embodiments, the AAV particle may have a targeting peptide inserted into the capsid to enhance tropism for a desired target tissue. It is to be understood that reference to the AAV particles of the disclosure also includes pharmaceutical compositions thereof, even if not explicitly recited.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of a gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Capsid: As used herein, the term “capsid” refers to the protein shell of a virus particle.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present disclosure, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo or payload.

Element: As used herein, the term “element” refers to a distinct portion of an entity. In some embodiments, an element may be a polynucleotide sequence with a specific purpose, incorporated into a longer polynucleotide sequence.

Encapsulate: As used herein, the term “encapsulate” means to enclose, surround or encase. As an example, a capsid protein often encapsulates a viral genome.

Engineered: As used herein, embodiments of the disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle (active ingredient) and an excipient, and/or an inactive ingredient.

Fragment: A “fragment,” as used herein, refers to a portion. For example, an antibody fragment may comprise a CDR, or a heavy chain variable region, or a scFv, etc.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology. Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of each of which are incorporated herein by reference in their entirety. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Insert: As used herein the term “insert” may refer to the addition of a targeting peptide sequence to a parent AAV capsid sequence. An “insertion” may result in the replacement of one or more amino acids of the parent AAV capsid sequence. Alternatively, an insertion may result in no changes to the parent AAV capsid sequence beyond the addition of the targeting peptide sequence.

Inverted terminal repeat: As used herein, the term “inverted terminal repeat” or “ITR” refers to a cis-regulatory element for the packaging of polynucleotide sequences into viral capsids.

Library: As used herein, the term “library” refers to a diverse collection of linear polypeptides, polynucleotides, viral particles, or viral vectors. As examples, a library may be a DNA library or an AAV capsid library.

Neurological disease: As used herein, a “neurological disease” is any disease associated with the central or peripheral nervous system and components thereof (e.g., neurons).

Naturally Occurring: As used herein, “naturally occurring” or “wild-type” means existing in nature without artificial aid, or involvement of the hand of man.

Open reading frame: As used herein, “open reading frame” or “ORF” refers to a sequence which does not contain a stop codon in a given reading frame.

Parent sequence: As used herein, a “parent sequence” is a nucleic acid or amino acid sequence from which a variant is derived. In certain embodiments, a parent sequence is a sequence into which a heterologous sequence is inserted. In other words, a parent sequence may be considered an acceptor or recipient sequence. In certain embodiments, a parent sequence is an AAV capsid sequence into which a targeting sequence is inserted.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained professional for a particular disease or condition.

Payload region: As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

RNAi agent: As used herein, the term “RNAi agent” refers to an RNA molecule, or its derivative, that can induce inhibition, interfering, or “silencing” of the expression of a target gene and/or its protein product. An RNAi agent may knock-out (virtually eliminate or eliminate) expression, or knock-down (lessen or decrease) expression. The RNAi agent may be, but is not limited to, dsRNA, siRNA, shRNA, pre-miRNA, pri-miRNA, miRNA, stRNA, lncRNA, piRNA, or snoRNA.

Sample: As used herein, the term “sample” or “biological sample” refers to a subset of its tissues, cells or component parts (e.g. body fluids, including but not limited to blood, serum, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a self-complementary viral genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides. e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called an siRNA duplex.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Targeting peptide: As used herein, a “targeting peptide” refers to a peptide of 3-20 amino acids in length. These targeting peptides may be inserted into, or attached to, a parent amino acid sequence to alter the characteristics (e.g., tropism) of the parent protein. As a non-limiting example, the targeting peptide can be inserted into an AAV capsid sequence for enhanced targeting to a desired cell-type, tissue, organ or organism. It is to be understood that a targeting peptide is encoded by a targeting polynucleotide which may similarly be inserted into a parent polynucleotide sequence. Therefore, a “targeting sequence” refers to a peptide or polynucleotide sequence for insertion into an appropriate parent sequence (amino acid or polynucleotide, respectively).

Target Cells: As used herein, “target cells” or “target tissue” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Vector: As used herein, the term “vector” refers to any molecule or moiety which transports, transduces or otherwise acts as a carrier of a heterologous molecule. In some embodiments, vectors may be plasmids. In some embodiments, vectors may be viruses. An AAV particle is an example of a vector. Vectors of the present disclosure may be produced recombinantly and may be based on and/or may comprise adeno-associated virus (AAV) parent or reference sequences. The heterologous molecule may be a polynucleotide and/or a polypeptide.

Viral Genome: As used herein, the terms “viral genome” or “vector genome” refer to the nucleic acid sequence(s) encapsulated in an AAV particle. A viral genome comprises a nucleic acid sequence with at least one payload region encoding a payload and at least one ITR

The present disclosure is further illustrated by the following non-limiting examples.

### EXAMPLES

### Example 1. Targeting Peptides Identified Using CREATE in Mice

The CREATE method for generating libraries of targeting peptides has previously been described in Deverman et al (Nature Biotechnology 34(2):204-209 (2016), the contents of which are herein incorporated in their entirety. This method was used to identify targeting peptides for enhancing AAV tropism to desired tissues (e.g., CNS). In short, random 7-amino acid peptides were inserted between amino acids 588 and 589 of K449R AAV9 (SEQ ID NO: 3), by inserting, at the DNA level, the nucleotides coding for random 7 amino acids into the corresponding region of the K449R AAV9 nucleotide sequence, to generate a DNA library. The K449R variant of AAV9 has the same function as wild-type AAV9. The DNA library was then used to create an AAV particle library which was intravenously administered to adult GFAP-Cre mice. DNAs packaged in AAV particles transducing the Cre-expressing cells were recovered in a Cre-dependent manner a week after administration. The recovered DNAs were cloned into the K449R AAV9 backbone to create a 2^{nd} round DNA library. A second round of in-vivo selection yielded a series of AAV variants comprising targeting peptides that enhanced tropism for CNS tissues. Examples of targeting peptides isolated in such a manner include targeting peptides PHP.B (TLAVPFK; SEQ ID NO: 43054), PHP.A (YTLSQGW; SEQ ID NO: 43055), PHP.B2 (SVSKPFL; SEQ ID NO: 43056), PHP.B3 (FTLTTPK; SEQ ID NO: 43057), PHP.S (QAVRTSL; SEQ ID NO: 43058), G2B-A7 (MNSTKNV; SEQ ID NO: 43060), and G2B5-G9 (VSGGHHS; SEQ ID NO: 43061) shown in Table 5 below.

TABLE 5
Exemplar Targeting Peptides

| Targeting | | | SEQ ID |
|---|---|---|---|
| Peptide | Sequence | Reference Information | NO: |
| PHP.B | TLAVPFK | WO2015038958 SEQ ID NO: 1 | 43054 |
| PHP.A | YTLSQGW | WO2015038958 SEQ ID NO: 60 | 43055 |
| PHP.B2 | SVSKPFL | WO2015038958 SEQ ID NO: 28 | 43056 |
| PHP.B3 | FTLTTPK | WO2015038958 SEQ ID NO: 29 | 43057 |
| PHP.S | QAVRTSL | WO2017100671 SEQ ID NO: 37 | 43058 |
| PRP.N | DGTLAVPFKAQ | WO2017100671 SEQ ID NO: 4 | 43059 |
| G2B4 (G2B-A7) | MNSTKNV | WO2017100671 SEQ ID NO: 43 | 43060 |
| G2B5 (G2B5-G9) | VSGGHHS | WO2017100671 SEQ ID NO: 44 | 43061 |
| PHP.B-EST | ESTLAVPFKAQ | WO2017100671 SEQ ID NO: 5 | 43062 |
| PHP.B-GGT | GGTLAVPFKAQ | WO2017100671 SEQ ID NO: 6 | 43063 |
| PHR.B-ATP | AQTLATPFKAQ | WO2017100671 SEQ ID NO: 7 | 43064 |
| PHP.B-ATT-T | ATTLATPFKAQ | WO2017100671 SEQ ID NO: 8 | 43065 |
| PHP.B-DGT-T | DGTLATPFKAQ | WO2017100671 SEQ ID NO: 9 | 43066 |
| PHP.B-GGT-T | GGTLATPFKAQ | WO2017100671 SEQ ID NO: 10 | 43067 |
| PHP.B-SGS | SGSLAVPFKAQ | WO2017100671 SEQ ID NO: 11 | 43068 |
| PHP.B-AQP | AQTLAQPFKAQ | WO2017100671 SEQ ID NO: 12 | 43069 |
| PHP.B-QQP | AQTLQQPFKAQ | WO2017100671 SEQ ID NO: 13 | 43070 |
| PHP.B-SNP(3) | AQTLSNPFKAQ | WO2017100671 SEQ ID NO: 14 | 43071 |
| PHPVB-SNP | AQTLAVPFSNP | WO2017100671 SEQ ID NO: 15 | 43072 |

The PHP.B 7-mer and flanking sequences were further evolved through site saturation mutagenesis of sets of three consecutive amino acids, using NNK codons, wherein N=any base and K is a G or T. DNA libraries of these site saturation mutagenesis sequences were generated to then create AAV particle libraries. CREATE was used for AAV particle selection in three different CNS cell populations, 1) astrocytes (GFAP-Cre), 2) GABA-ergic (inhibitory) neurons (VGAT-IRES-Cre), and 3) a subset of glutamatergic (excitatory) neurons (Vglut2-IRES-CRE). AAV particles transducing Cre-expressing cells were subjected to two rounds of selection and were then recovered and assessed by clonal and/or next generation sequencing (NGS). An example of a targeting peptide isolated in such a manner include PHP.N (DGTLAVPFKAQ; SEQ ID NO: 43059), PHP.B-EST (ESTLAVPFKAQ; SEQ ID NO: 43062), PHP.B-GGT (GGTLAVPFKAQ; SEQ ID NO: 43063), PHP.B-ATP (AQTLATPFKAQ; SEQ ID NO: 43064), PHP.B-ATT-T (ATTLATPFKAQ; SEQ ID NO: 43065), PHP.B-DGT-T (DGTLATPFKAQ; SEQ ID NO: 43066). PHP.B-GGT-T (GGTLATPFKAQ; SEQ ID NO: 43067), PHP.B-SGS (SGSLAVPFKAQ; SEQ ID NO: 43068), PHP.B-AQP (AQTLAQPFKAQ; SEQ ID NO: 43069), PHP.B-QQP (AQTLQQPFKAQ; SEQ ID NO: 43070). PHP.B-SNP(3) (AQTLSNPFKAQ; SEQ ID NO: 43071), and PHP.B-SNP (AQTLAVPFSNP; SEQ ID NO: 43072) as shown in Table 5 above.

### Example 2. Identification of Improved CNS Targeting Peptides and AAV Particles

Using CREATE in mice to isolate novel targeting peptides for enhancing tropism of AAV particles to the CNS after intravenous delivery has proven useful in identifying targeting peptides and associated AAV variants such as PHP.B and PHPN. These vectors have shown broad CNS distribution following intravenous administration when tested in mice, however, whether these vectors will show similar efficacy in NHP or humans remains to be determined. Even if efficacy is the same, the dose necessary for CNS distribution after IV administration in a large mammal may be prohibitive. Continuing to evolve targeting peptides in systems more closely matched to humans (e.g., NHP) can help generate targeting peptides and AAV particles with more desirable CNS transduction profiles, for use in humans using intravenous delivery.

### Pilot Study to Test Co-Transduction of AAV-Cre Vectors and PHP.N Reporter

To use CREATE in non-transgenic NHP lacking Cre expression, AAV-Cre vectors need to be delivered intraparenchymally to the target CNS tissues of the NHP. To evaluate co-transduction of intraparenchymally (IPa) administered Cre-expressing AAV particles and an intravenously administered Cre-dependent reporter packaged in an AAV particle, the system will first be tested in mice.

Four Cre-vectors will be created, 1) for ubiquitous expression using a CAG, UBC or EF1α promoter (CAG- UBC- or EF1α-Cre), 2) for neuronal expression using a human synapsin promoter (hSyn1-Cre), 3) for astrocytic expression using a truncated GFAP promoter (GfAB₁D-Cre, and 4) for oligodendrocyte expression using a myelin basic promoter (MBP-Cre), then packaged into an AAV capsid (AAV1, AAV9, AAVrh10 or AAVPHP.B) to generate AAV particles. A vector expressing a fluorescent protein can be used to mark vector spread at the injection site (e.g., CAG-mRuby2/tdTomato).

Two reporter AAV particles will be created for testing, 1) AAVPHP.N:CAG-DIO-mTurquoise2 (forward)/GFP(reverse) and 2) AAV1/9/rh10-CAG-DIO-mTurquoise2(f)/GFP(r). These reporter AAV particles will be delivered either by intravenous administration or by co-intraparenchymal administration with the Cre-vectors. Only those cells of the CNS also expressing Cre (co-transduced by a cre-vector administered intraparenchymally) will express the reporter.

Each cre-vector will be injected into two mice and each mouse will also be administered one of the two reporter AAV particles (16 mice). The reporter AAV particles may be administered intravenously or intraparenchymally. Four mice will serve as non-Cre mRuby2/tdTomato controls. Using an intracranial surgical approach, Cre-vectors (and/or a reporter AAV) will be intraparenchymally administered to the striatum, thalamus and/or cortex at a volume of 0.5 μL and a concentration of 3×10¹⁰ to 3×10¹⁰ vg/site. The reporter AAV particles administered intravenously will be given at a concentration of 3×10¹¹ vg.

Mice will be evaluated three weeks after AAV particle administration. Co-transduction efficiency will be evaluated based on imaging of the coincidence of green and red fluorescent signals.

It is anticipated that the IV-administered reporter AAV particles will deliver the reporter to CNS tissues expressing Cre and mRuby2/tdTomato (e.g., striatum, thalamus and/or cortex). To be considered successful, the results of this pilot experiment will show a high yield of GFP (reporter) transduced neurons in the mRuby/tdTomato transduced brain region (delivered by IPa). As an example, >50% of neurons and astrocytes and 10-30% of oligodendrocytes in the mRuby/tdTomato transduced brain region may also express GFP.

It is possible that off-target cell-types will be transduced by the Cre-vectors and reporter AAV particles. In this case, miRNA binding sites complementary to miRNAs expressed in these off-target cell types, but not in the target cell type, may be introduced into the viral genomes of the AAV particles for enhanced targeting and specificity.

### Pilot Mouse AAV-Cre Based AAV-PHP.B/N Library Selection

In order to test whether expected and/or novel capsid sequences comprising targeting peptides can be recovered from the brain following intraparenchymal delivery to the striatum, thalamus and/or cortex of the Cre-vectors and intravenous delivery of an AAV capsid library, a pilot study will first be performed in mice. This study can also be used to test library diversification/randomization strategies.

Four DNA libraries based on PHP.B/PHP.N will be generated by PCR, with mixed bases at the desired positions. PCR fragments will be cloned into the library acceptor AAV genome (e.g., AAV9) by Gibson assembly and processed to remove unassembled DNA. The AAV DNA libraries will then be transfected directly into HEK293 cells for AAV particle production. The libraries will be built into an AAV vector with rep/cap in cis and a floxed element. Libraries of AAV particles will be generated based on these DNA libraries.

Library 1 will extend the targeting peptide sequence to include the 2-3 amino acids immediately upstream and/or downstream of the 7-mer peptide insert. These amino acids will be replaced with random amino acids generated by NNK-containing primers. This library will comprise site saturation mutagenesis at 5-6 amino acids of the targeting peptide sequence and produce a library diversity of 3.2×10⁶ to 6.4×10⁷ sequences.

Library 2 will be a mixed library of PHP.B/N. PHP.B2 and PHP.B3 targeting peptides mutated 3-amino acids at a time, creating 21 combined libraries with approximately 1.6×10⁵ variants. This library is based on the same strategy as was used for generation of PHP.N from PHP.B, described above.

Library 3 will be a mixture of two novel 7-mer peptide libraries inserted between amino acids 588 and 589 of AAV9 (SEQ ID NO: 2), K449R AAV9 (SEQ ID NO: 3) and/or AAV-DJ8 and/or AAV5.

Library 4 will include capsid sequences modified at a distinct surface site (e.g., loop IV and/or VIII).

Cell-type specific Cre-vectors (as described above) and AAV particle libraries will be injected into mice (2 mice per Cre-vector, per library and 2 Cre-control mice; 18 mice total). Cre-vectors will be delivered to the striatum and cortex via intraparenchymal administration, whereas the AAV particle libraries will be administered intravenously.

Libraries may optionally be spiked with a small amount of AAV9 or AAVPHP.B to serve as a normalization control.

Tissue samples (e.g., brain) will be collected 3 weeks after administration of Cre-vectors and AAV libraries. The total DNA will be isolated and capsid sequences recovered. For recovery. Cre-dependent amplification strategies may be used. Capsid sequences will be prepared for NGS using standard methods known in the art.

DNA libraries will be sequenced by NGS at a depth of 50M, 100 bp reads. Virus libraries will be sequenced at a depth of 20M, 100 bp reads. Samples recovered from one CNS region and one liver sample per mouse will be sequenced at a depth of 5M reads.

Capsid sequences comprising targeting peptides that show evidence of enrichment across CNS cell-types will be isolated and cloned. If no clearly enriched variants arise from the first round library, a second-round capsid library may be generated. Capsid sequences comprising targeting peptides that show evidence of enrichment across CNS cell-types from a second round library will be isolated and cloned.

Isolated variants will be screened in adult mice. Each capsid variant will be used to package a ubiquitous reporter vector (e.g., ssAAV-CAG-GFP) and transduction efficiency to CNS tissues (brain and spinal cord) subsequent to intravenous administration will be evaluated by immunofluorescence imaging, as compared to parental capsids PHP.B, PHP.N and/or AAV9. Vector genome quantification in target tissues (e.g., brain, spinal cord, liver, heart, spleen, skeletal muscle) will be determined by qPCR.

It is anticipated that intraparenchymal delivery of AAV-Cre vectors will allow for the adaptation of the CREATE method to non-transgenic animals. A successful outcome of this pilot study would be the identification of one or more capsid variants (and targeting peptides) with enhanced tropism for CNS tissue after intravenous administration. As a positive control, it is anticipated that PHP.B, PHP.N, PHP.B2, and PHP.B3 or other AAV variants (see Table 5) will be identified again in Library 2.

### Pilot Testing of NHP Injection Strategy

AAV-Cre vectors will need to be delivered directly to the CNS tissue of the non-transgenic NHP. The injection strategy will be validated with a pilot study in NHP. The pilot study will assess spacing of injection sites for separation between areas of vector spread based on differing promoters (promoters described above). Further, the pilot study will address toxicity or immune responses to the modified CREATE system, by assessing markers of cell death, glial reactivity (Iba1 and GFAP immunostaining), T cell infiltration, and/or H&E staining. Simultaneously, expression of IV administered AAV-PHP.B delivered at a concentration of about 2×10¹³ vg/kg can be assessed for transduction efficiency based on the Cre-dependent reporter. Transduction efficiency of AAV particles comprising reporter constructs delivered by co-intraparenchymal administration may also be determined.

Selection of AAV capsid variants in NHP

AAVPHP.B/N based capsid libraries are generated as described above, however prior to injection into NHP, the libraries will be titred and assessed by NGS. Cell-type specific Cre-vectors will be stereotactically delivered to the CNS tissue of three NHP, while the AAV capsid libraries will be injected intravenously, one week prior to injection of Cre vector. Stereotactic delivery to CNS tissue, may include intraparenchymal delivery to the putamen, thalamus, cortex, hippocampus and/or deep cerebellar nuclei.

Two-three weeks post-Cre-vectors stereotactical administration, tissue samples (e.g., brain, spinal cord, peripheral organs) are collected for analysis. Total vector DNA is isolated from the desired tissue sample(s) and a Cre-dependent amplification strategy is used to recover capsid sequences from each animal and each tissue region of interest. Samples are then prepared for NGS and sequenced as described previously.

AAV capsid variants showing enrichment across CNS cell types will be cloned for further examination and tested in adult mice/NHP for transduction efficiency to tissues of interest.

It is anticipated that AAV capsid sequences comprising targeting peptides with enhanced tropism to CNS tissues after IV administration, as compared to AAV9, PHP.B or PHP.A will be identified through these NHP experiments.

The methods for selection of AAV capsid variants in NHP as described above can be repeated, and a second round library generated to further narrow the number of lead AAV capsid sequence candidates with enhanced tropism to the CNS and/or to enhance reliability. AAV capsid variants showing enrichment across CNS cell types in the second round library will also be cloned for further examination and testing in mice/NHP.

A second evolution step may optionally be included for the AAV capsid candidates generated in the aforementioned NHP selection process. AAV capsid variant sequences with targeting peptides inserted at amino acid position 588-589 of the parent capsid sequence, are further modified at a second surface site. Modifications at more than one surface site may increase CNS tropism of the AAV capsid variants.

The top ten AAV capsid variants identified in these experiments will be cloned for further testing, and a viral genome encoding a reporter construct will be packaged inside. Variants will be identified based on total read number recovered from each sample, enrichment over the previous round or the starting library, and predicted high level of virus production. AAV particles will be titred and injected IV into adult mice at a dose of 1×10¹¹ vg/mouse. Three weeks after injection, the mouse is perfused and tissues collected and processed for assessment of fluorescence by confocal microscopy. The top three candidates after further examination in mice will be tested individually in NHP to identify an AAV variant capsid with the best NHP CNS transduction profile following IV administration. Alternatively, AAV particles may be tested immediately in NHP and/or in parallel with the mouse studies described above.

### Example 3. Identification of Dorsal Root Ganglion Targeting Peptides and AAV Particles

The dorsal root ganglia are clusters of neuronal cell bodies that lie just outside the spinal cord along the dorsal roots. Neurons of the dorsal root, with cell bodies in the DRG, convey sensory information from the periphery back to the spinal cord and central nervous system. Neurons of the DRG can be difficult to target with efficiency and selectivity for delivery of, for example, a therapeutic agent. Pathological conditions associated with DRG and their component neurons (e.g., neuropathic pain) could potentially be more easily treated if targeting peptides and associated AAV particles are identified for specific and efficient transduction of DRG following intravenous administration of AAV particles.

### Mouse Sensory Neuron Selection, First Round of Evolution

One of the targeting peptides (PHP.S; SEQ ID NO: 43058) identified using CREATE in mice (as described above), has previously demonstrated efficient transduction of peripheral neurons of the dorsal root, nodose, sympathetic, parasympathetic and cardiac ganglia. To identify targeting peptides and associated AAV particles with enhanced specificity for neurons of the DRG and subpopulations thereof, libraries based on this PHP.S targeting peptide will be generated and screened in transgenic mice.

Tree AAV capsid libraries will be created and screened. Each library will be generated by site-saturation mutagenesis of different subsets of amino acids of the parent PHP.S amino acid sequence or flanking sequences of the parent AAV capsid.

Library 1 will involve site-saturation mutagenesis of the amino acids flanking the PHP.S 7-mer on both the 5′ and 3′ end in the parent AAV capsid sequence. This mutagenesis may include 4 or 5 amino acids flanking the 7-mer, yielding approximately 1.6×10⁵ to 3.2×10⁶ unique sequences.

For Library 2, sets of three amino acids of the PHP.S 7-mer and/or flanking regions will be evolved together, to generate six, 3 amino acid libraries with approximately 4.8×10⁴ unique amino acid sequences.

Library 3 will be a mixture of two novel 7-mer peptide libraries inserted between amino acids 588 and 589 of AAV9 (SEQ ID NO: 2), K449R AAV9 (SEQ ID NO: 3) and/or AAV-DJ8, giving approximately 1.3×10⁹ unique amino acid sequences each.

The three DNA libraries will be used to generate AAV particle libraries. Both DNA and AAV particle libraries will be titered by qPCR and assessed by NGS. AAV particle libraries will be given to transgenic mice via intravenous administration at a dose of approximately 1×10¹¹ vg/mouse.

Five transgenic mouse lines will be used, as follows; 1) Vglut-Cre for selection of targeting peptides for total sensory neuron transduction, 2) Nav1.8 (Scn10a)-Cre for selection of targeting peptides for nociceptive neuron transduction, 3) Parvalbumin (PV)-Cre for selection of targeting peptides for parvalbumin neuron transduction, 4) TH-Cre for selection of targeting peptides for sympathetic/adrenergic neuron transduction and 5) Chat-Cre for selection of targeting peptides for parasympathetic/cholinergic neuron transduction.

One to two weeks after intravenous delivery, AAV particles will be recovered from neurons of the dorsal root, nodose, and cardiac ganglia, sympathetic and parasympathetic neurons including those of the myenteric ganglia and submucosal plexus of the intestines, and olfactory sensory neurons. DNA and AAV particle libraries associated with a desired transduction profile (e.g., transduction of a particular neuronal subset) will be sequenced and further characterized.

Fluorescence imaging will be used to determine transgene expression in the DRG and other peripheral ganglia. Immunostaining can be used to co-localize transgene expression to particular neuronal subtypes of the DRG. Transgene/reporter expression can be driven by either ubiquitous or neuron-specific promoter(s).

It is anticipated that several rounds of evolution may be necessary in order to identify a targeting peptide and associated AAV particle with a transduction profile for a specific neuronal subset, such as nociceptive neurons of the DRG. Negative selection may also be applied, wherein targeting peptides and associated AAV particles are eliminated from consideration if the transduction profile is considered undesirable.

### Mouse Sensory Neuron Selection, Second Round of Evolution

One or more of the targeting peptides with enhanced specificity and efficiency of transduction of neurons of the DRG will be further evolved to increase specificity. Evolution strategies may include site-saturation mutagenesis of loop IV (e.g., amino acids 452-458) or mutation of known receptor binding sites.

Fluorescence imaging will be used to determine transgene expression in the DRG and other peripheral ganglia. Immunostaining can be used to co-localize transgene expression to particular neuronal subtypes of the DRG. Biodistribution of vector genomes can be determined by qPCR.

### Example 4. Targeting of AAVPHP.B and AAVPHP.A to the CNS and DRG

To determine the tropism of AAV particles comprising the PHP.B or PHP.A targeting peptide to CNS and to the dorsal root ganglia after intravenous administration, the following study design was executed in adult (6-7 weeks) male C57BL/6 mice.

TABLE 6
Study design for PHP.B/PHP.A targeting to DRG

| | | Route | Volume | N | End of |
|---|---|---|---|---|---|
| Test Article | Dose (vg) | (Tail vein) | (μL) | (IHC) | Study |
| Vehicle | 0 | IV | 160 | 2 | D28 |
| AAV9-ssGFP | 5 × 10¹¹ | IV | 160 | 4 | D28 |
| PHP.B-ssGFP | 5 × 10¹¹ | IV | 160 | 4 | D28 |
| PHP.A-ssGFP | 5 × 10¹¹ | IV | 160 | 4 | D28 |

Targeting peptides PHP.B (TLAVPFK; SEQ ID NO: 43054) or PHP.A (YTLSQGW; SEQ ID NO: 43055) were inserted into an AAV9 (SEQ ID NO: 2) capsid sequence between amino acid positions 588 and 589. AAV9 without a targeting insert was used as a comparison vector, and PBS used as a control. A single stranded viral genome encoding GFP was used, yielding AAV particles AAV9-ssGFP, PHP.B-ssGFP and PHP.A-ssGFP. AAV particles (or PBS) were administered intravenously via the tail vein at a dose of 5×10¹¹ and volume of 160 μL.

Twenty eight days after intravenous delivery of the AAV particles, the animals were sacrificed and tissues collected for immunohistochemical and immunofluorescent analyses. Tissues from the dorsal root ganglion, brain (striatum, cortex, hippocampus), spinal cord (ventral horn), liver, heart and muscle were collected for processing by standard methods known in the art.

Analysis of tissue sections of brain, striatum, cortex, hippocampus and ventral horn showed that vectors comprising PHP.B or PHP.A targeting peptides mediate greater gene delivery throughout the CNS than AAV9 following intravenous injection in adult mice.

Similar analysis of tissue sections of the DRG demonstrated that after intravenous administration of the AAV particles, the greatest transduction of sensory neurons (most GFP signal) was seen with the use of targeting peptide PHP.B. Both AAV9 and PHP.A yielded transduced sensory neurons in the DRG following intravenous delivery, though to a lesser extent to that seen with PHP.B. As expected, intravenous delivery of PBS showed no transduction of sensory neurons of the DRG. These results demonstrate that targeting peptide PHP.B can be used for enhanced targeting to the CNS and of the sensory neurons of the dorsal root ganglia.

### Example 5. Seven-Mer Targeting Peptide Libraries for Selection in NHP

DNA libraries and associated AAV capsid libraries of 7-mer targeting peptides were generated and tested in mice and NHP. Targeting peptides were inserted into an AAV9 (SEQ ID NO: 2) parent capsid sequence between amino acid positions 588 and 589 (loop VIII).

AAV1-Cre vectors were administered intraparenchymally to the striatum and thalamus of three adult (2-11 yr, 4-8 kg) non-human primates (Macaca mulatta or rhesus macaque), while the AAV capsid library was administered intravenously as shown in the study design of Table 7, below. The AAV particles were delivered in an excipient comprising 200 mM NaCl, 1 mM KH₂PO₄, 3 mM Na₂HPO₄, 0.001% Pluronic F-68 at pH 7.4.

TABLE 7
Study design for AAV9-7mer library in NHP

| | | | | Day 11 ± 1 Dosing (MRI-CED) | | | | |
|---|---|---|---|---|---|---|---|---|
| | | Day 1 Dosing (IV) | | | Dose & Volume | | | |
| | | | Dose & | | Putamen | | Thalamus | |
| Group | N | Material | Volume | Material | Left | Right | Left | Right |
| 1A | 1 | AAV9-7mer | 5.0 × 10¹² vg/kg | AAV1-Cre | 3.75 × 10¹¹ vg | 7.5 × 10¹⁰ vg | 6.25 × 10¹¹ vg | 1.25 × 10¹¹ vg |
| | | | (1 mL/kg) | | (150 μL) | (150 μL) | (250 μL) | (250 μL) |
| 1B | 1 | AAV9-7mer | 5.0 × 10¹² vg/kg | AAV1-Cre | 7.5 × 10¹⁰ vg | 1.5 × 10¹⁰ vg | 1.25 × 10¹¹ vg | 2.5 × 10¹⁰ vg |
| | | | (1 mL/kg) | | (150 μL) | (150 μL) | (250 μL) | (250 μL) |
| 1C | 1 | AAV9-7mer | 5.0 × 10¹² vg/kg | AAV1-Cre | 3.75 × 10¹¹ vg | 1.5 × 10¹⁰ vg | 6.75 × 10¹¹ vg | 2.5 × 10¹⁰ vg |
| | | | (1 mL/kg) | | (150 μL) | (150 μL) | (250 μL) | (250 μL) |

Five animals were selected based on results of neutralizing antibody assays for anti-AAV antibodies to AAV1 and AAV9. Minimal or no detectable capsid specific antibodies was required for selection. Two of these five animals served as alternate study animals. Study subjects underwent a pre-project physical examination.

On Day 1 of the study, each animal received intravenous infusion of the AAV9-7mer library. On Day 7±1, animals had bilateral intracranial infusion of AAV1-Cre vectors to the putamen and thalamus, using MRI-guided convection enhanced delivery.

On the day of dosing, AAV9-7mer libraries or AAV1-Cre containing compositions were thawed at 2-8 C. The excipient detailed above was used as a vehicle control. Dosing solutions were gently centrifuged to collect material from the vial cap then transferred to the syringe/needle for delivery. Dosing solutions were kept at room temperature until administration, but not longer than for 2 hours. On Day 7±1, for the MRI-CED dosing, Gadoteridol (ProHance, Bracco Diagnostics Inc) was added at a 1:250 ratio and mixed carefully by tube inversion prior to loading into dosing syringes.

For intravenous administration of the AAV9-7mer libraries on Day 1, animals were immobilized with ketamine (10 mg/kg, administered intramuscularly (IM)) and dexamedetomidine (15 μg/kg, IM) and given a single intravenous infusion (dose volume of 1 mL/kg) via the saphenous vein, using a syringe pump. After dosing, a 0.2 mL flush of vehicle/excipient was administered. Animals were allowed to recover and returned to home cages.

On Day 7±1, for the MRI-CED of AAV1-Cre vectors, animals were anesthetized with ketamine (10 mg/kg) and dexmedetomidine (15 μg/kg) via intramuscular injection, weighed, shaved on the head and neck, intubated and maintained on 1-5% isoflurane. The head was secured in a sterotaxic frame and the overlying skin cleaned and prepared for implantation procedures. Aseptic techniques were used to expose the skull by opening the wound site by anatomical layers. Bilateral craniotomies were performed at entry sites located above the frontal/parietal and occipital lobes. A skull mounted cannula guide ball array was temporarily secured to the skull with titanium screws over each burr hole. After implantation of the cannula guides, animals were transferred to the MRI suite. MRI was used to align the cannula guides with putamen and thalamus targets ipsilateral (same side of the brain/body) to each cannula guide. Repeat MRI was used to visually monitor infusions.

Dosing solutions were delivered via an adjustable tip 16 G cannula (MRI Interventions Inc) primed with the dosing solution then guided into each target site through the skull mounted cannula arrays. Cannula were connected via microbore extension lines (Smiths Medical) to a syringe mounted on a syringe pump (Harvard apparatus). Ascending infusion rates (up to 10 μL/min) were used to deliver dose volumes (150 μL/putamen; 250 μL/thalamus) to the putamen and thalamus. Serial MRI scans were used to monitor infusate distribution.

Immediately after MRI-CED dosing, animals were transferred back to the operating room where the cannula guide system was removed and the wound site closed, using a vicryl suture. Animals were provided pre- and post-operative medications, allowed to recover from anesthesia and returned to their home cages.

Fourteen days after MRI-CED dosing to the putamen and thalamus (Day 212) animals were euthanized and tissue samples collected. Euthanasia was performed using intravenous delivery of a pentobarbital sodium solution (100 mg/kg), followed by a bilateral thoracotomy. A transcardial perfusion using ice-cold saline was performed via left cardiac ventricle, with approximately 2.5 L of ice-cold saline with heparin (100 U/mL) using a perfusion pump set to 200-400 mL/min over about 10 min. Samples were collected from brain, spinal cord, 12 dorsal root ganglia and associated roots (4 each from cervical, thoracic and lumbar spinal cord), liver, heart (left ventricle and right atrium), gastrocnemius muscle, soleus muscle, pancreas, kidney, spleen, lung, adrenal glands, stomach, sciatic nerve, saphenous nerve, thyroid gland, eyes (with optic nerve), pituitary gland, skeletal muscle (rectus femoris), colon, duodenum, ileum, jejunum, skin of the leg, superior cervical ganglia, urinary bladder, ovaries, uterus, prostate gland, testes, and/or any sites identified as having a lesion, and fresh frozen for biochemical analysis.

At the time of collection, all samples were photographed alongside a ruler for scale. The brain was cut coronally in a matrix at 3 mm slice thickness, then alphabetically annotated rostral (nose) to caudal (tail). A small punch (1 mm diameter) was created in the white matter of the right cortex for orientation purposes. Brain samples were frozen on dry ice. The spinal cord was cut into segments corresponding to each vertebral level, then transferred to a chilled cryovial tube and snap frozen on dry ice. DRG and associated roots were collected from C1, C3, C5, C7, T3, T5, T7, T9, L1, L3, L5 and L7. These were transferred into a chilled cryovial and frozen on dry ice. For the other organ samples collected, two samples per organ, approximately 1.0×1.0×0.5 cm (or the entire organ if smaller) were collected, transferred to a chilled cryovial tube and snap-frozen on dry ice. All samples were stored frozen at −60 C or below.

Blood and cerebrospinal fluid (CSF) samples were collected on Day 1, prior to IV dosing, on Day 7±1, prior to MRI-CED dosing, and on Day 21±2 prior to termination. Time points and descriptions of sample collection are shown below in Table 8. Whole blood samples were collected in tubes containing EDTA, mixed by inversion and aliquoted into 2 sub-samples, then stored at −60 C or below. Sample volumes listed in the Serum column, refer to the volume of whole blood which was then processed to serum and split to 2 aliquots and stored at −60 C or below. CSF samples were collected after animals were immobilized with ketamine (10 mg/kg; IM) and dexmedetomidine (15 ug/kg; IM). The neck was shaved and flexed and skin prepared. A 23 G needle was manually advanced through the skin and the atlanto-occipital membrane into the cerebellomedullary cistern space. Once CSF flow was confirmed in the needle hub, CSF collection began. Collected CSF was centrifuged (4,500 g for 5 min at 4 C), the supernatant filtered through a 0.8 μm low protein binding syringe filter, and the filtrate split into 4 aliquots for analysis. At necropsy, all available CSF was collected, processed, and aliquoted to approximately 1.0 mL samples. CSF samples were stored at −60 C or below.

TABLE 8
Time points for sample collection

| | | | Sample Volume (mL) | | |
|---|---|---|---|---|---|
| | | | | Whole | |
| Time point | Fasted | N | Serum | blood | CSF |
| Pre-screening | Yes | 18 | 2.0 | N/A | N/A |
| Day 1: prior to | Yes | 3 | 2.0 | 0.5 | 1.5 |
| IV dosing | | | | | |
| Day 7 ± 1: prior | Yes | 3 | 2.0 | 0.5 | 1.5 |
| to intraparenchymal | | | | | |
| MRI-CED dosing | | | | | |
| Day 21 ± 2: prior | Yes | 3 | 2.0 | 0.5 | All |
| to necropsy | | | | | available |

Animals were monitored throughout the study with twice daily clinical observations assessing mortality, neurological signs or other abnormalities and/or signs of pain or distress. Food consumption was also monitored daily. Body weights were measured once weekly, beginning prior to IV dosing on Day 1.

After this first round of selection in NHP, targeting peptides with amino acid sequences as represented by SEQ ID NO: 4-14326 (shown in Table 2) were recovered from CNS tissues. DNA libraries comprising SEQ ID NO: 14327-42972 have been prepared for second round selection in NHP.

### Example 6. Three-Mer Targeting Peptide Library for Selection in NHP

A DNA library and associated AAV capsid library was generated based on a 9-mer of the PHP.N targeting peptide. The 9-mer PHP.N amino acid sequence was further evolved through site saturation mutagenesis of sets of three consecutive amino acids, to generate the targeting peptides represented by SEQ ID NO: 42973-42999, as shown in Table 3. Amino acids at positions 1, 2, and 6-9 of the parent PHP.N 9-mer remained the same, with site saturation mutagenesis of only amino acids at positions 3, 4, and 5, yielding 27 targeting peptide sequences for screening and characterization.

### Example 7. Selection of Targeting Peptides Using in Mice

Experiments were performed using methods described in Example 1 where AAV9 7-mer libraries were injected at 3×10¹¹ vg/animal into hSyn1-Cre or GFAP-Cre mice (3 mice per group). Two weeks after the injection, brains were harvested and DNA was extracted by whole genomic DNA isolation method or Trizol method. Capsid sequences were recovered and prepared for NGS using standard methods known in the art. Approximately 460 high read count sequences were recovered from the hSyn1-Cre mice and around 240 high read count sequences were recovered from the GFAP-Cre mice. The whole genomic DNA and Trizol methods both resulted in similar average number of recovered sequences overall, however, the Trizol method resulted is more sequences recovered with a small number of individual PCR reactions.

### Example 8. Murine Co-Transduction of AAV-Cre and PHP Studies

To evaluate the efficacy of co-transduction of intrastriatally (IS) administered Cre-expressing AAV particles and an intravenously administered Cre-dependent reporter packaged in an AAV particle for use in NHP studies, the system was tested in mice. Four Cre-vectors were created, 1) for ubiquitous expression using a CAG, UBC or EF1α promoter (CAG- UBC- or EF1α-Cre), 2) for neuronal expression using a human synapsin promoter (hSyn1-Cre), 3) for astrocytic expression using a truncated GFAP promoter (GfAB₁D-Cre, and 4) for oligodendrocyte expression using a myelin basic promoter (MBP-Cre), then packaged into an AAV capsid (AAV1, AAV9, AAVrh10 or AAVPHP.B) to generate AAV particles. A vector expressing a fluorescent protein was used to mark vector spread at the injection site (e.g., CAG-mRuby2/tdTomato). The AAV-PHP.N:BG switch reporter was used as the reporter AAV.

On day 1, the reporter AAV particles (AAV-PHP.N:Bgswitch) were injected intravenously into the mice at a dose of 5×10¹² vg/kg body weight (1×10¹¹ vg per 20 g mouse). On day 6, a mix consisting of Cre-vector and reporter AAV particles were intrastriatally (IS) injected into mice at anteroposterior (AP) +0.5 from Bregma, mediolateral (ML) +2.0, dorsoventral (DV) −3.8. Three microliters of the virus mix was injected at a rate of 0.5 μl/minute at three different doses namely 1×10¹⁰, 2×10⁹ or 4×10⁸/site, respectively. Following deliver the needle was raised −2.8 mm and 5 minutes later, the needle was raised slowly and removed.

On day 20, mice were euthanized and a trans-cardiac perfusion was performed using 4% PFA (paraformaldehyde) in 1×PBS. CNS tissue was harvested and tissue sections were analyzed by immunofluorescence for the localization of green and red fluorescent signals. Expression of the reporter indicates co-transduction of the CNS cells by the reporter and the Cre. GFAP-Cre appeared to more efficiently turn off the expression of the blue-green switch vector when compared to the hSyn1-Cre. High dose Cre (1×10¹⁰ vg/site) caused local reduction of reporter expression around injection site which was not observed with the mid (2×10⁹ vg/site) or low dose (4×10⁸ vg/site). Therefore mid to low doses of Cre may be more suited for reducing localized toxicity. The spread of reporter expression beyond the injection was also found to be dose dependent with the high dose Cre injection demonstrating the largest spread of reporter expression.

### AAV-Cre Based AAV1-PHP.N Library in Mice

A pilot study was performed to test whether expected and/or novel capsid sequences containing targeting peptides could be recovered from the brain following intraparenchymal delivery to the striatum, thalamus and/or cortex of the Cre-vectors and intravenous delivery of an AAV capsid library.

A mixture of two different libraries was injected intravenously into mice. The mixture included an AAV9 7-mer library (1×10¹¹ vg/animal), like that described in Table 2 and Example 5, and a library of PHP.N (PHP.eB) targeting peptides mutated 3-amino acids at a time (1×10⁹ vg/animal), like that described in Table 3 and Example 6 above. Cell-type specific Cre-vectors namely AAV1-GfAB₁D-Cre and AAV1-hSyn1-Cre were injected at a dose of 4×10⁸ vg/site and 2×10⁹ vg/site respectively to 2 mice per group via intraparenchymal administration.

Tissue samples from the brain, such as, but not limited to, striatum, cortex, and thalamus were collected 3 weeks after administration of Cre-vectors and AAV libraries. The total DNA was isolated and capsid sequences recovered and sequenced using NGS.

34 unique AAV-PHP.N-based sequences were recovered from the NGS analysis. All recovered PHP.N sequences were unique at the nucleotide level. Several amino acid sequences (encoded by different nucleic acid sequences) were represented more than once in the sequence results, indicating their enrichment. As predicted, targeting peptides with T or S at position 1 within the 3-mer were strongly selected. 70 unique AAV9-7mer sequences were also recovered Fewer unique amino acid sequences were recovered with the Cre co-transduction as compared to the studies with the Cre transgenic mice. The parent AAV-PHP.N sequence was not reselected.

### Example 9. Identification of Targeting Peptides Directed to Dorsal Root Ganglia and Trigeminal Ganglia

The first round of evolution of peptides targeting the DRGs was performed as described in Example 3. The three libraries described in Example 3 were injected at the following dose: PHP.S: 1.6×10⁹ and 6×10⁸; AAV9 7-mer and AAV-DJ/8 7-mer: 1.9×10¹¹. One to two weeks after intravenous delivery, AAV particles were recovered and DNA from the Cre lines in Table 9 were sequenced. Table 9 also provides the number of sequences recovered from AAV9, AAV-DJ8 7-mer, and AAV-PHP.S variants.

TABLE 9
No. of unique sequences retrieved

| Cre line | AAV9 | AAV-DJ8 | PHP.S |
|---|---|---|---|
| Vglut2 | 267 | 88 | 3 |
| Nav1.8 | 122 | 41 | 1 |
| TH | 243 | 96 | 6 |
| Vglut1 | 37 | 23 | 0 |
| Total | 669 | 248 | 10 |

AAV-PHP.S variants recovered from the first round of evolution included SEQ ID NO: 43073-43082. AAV9 and AAV-DJ8 based libraries were selected for a second round of evolution due to the higher number of unique sequences retrieved from these libraries. For the second round, 8 copies of each variant recovered from round 1 were synthesized. The second round of selection was performed in 5 mice Cre lines, namely. PV-Cre, Nav1.8 (Scn10a), TH-Cre, Vglut1 and Vglut2, using wild-type C57BL6/J mice as a control. Each group included two mice which were given an intravenous viral dose of 2×10¹¹ vg/mice (1×10¹¹ vg/library). 10 days after the IV injection, tissues from 3 Cre lines (PV-Cre, Nav1.8 Cre, and TH-Cre) as well as wild-type C57BL6/J were harvested. Dorsal root ganglia (DRG) and terminal ganglia (TG) were processed and AAV9-7-mer viral DNA and its flanking sequence were recovered by PCR and assessed by next-generation sequencing. The sequencing data were analyzed and SEQ ID NO: 43342-43691 were recovered. Variants that showed high enrichment score and those that were represented in more than one library or tissue were selected for further analysis. The following variants were cloned into pUC57mini-iCap-PHP.B1.2 backbone for further analysis: Variant #1:

(SEQ ID NO: 43635)
(GCTCAGGCTGCTGTTAGTAGTATTCCTGCTCAG;
Amino acid sequence:
(SEQ ID NO: 43285)
AQAAVSSIPAQ;
Variant #2:
(SEQ ID NO: 43373)
(GCGCAGGGGGCGGATTTGAGGAATCCGGCGCAG;
Amino acid sequence
(SEQ ID NO: 43094)
AQGADLRNPAQ;
Variant #3
(SEQ ID NO: 43609)
(GCCCAAGAGGCGTCRIGGAATGCTCGGGCACAG;
Amino acid sequence:
(SEQ ID NO: 43259)
AQEASGNARAQ
Variant #4:
(SEQ ID NO: 43506)
(GCTCAGCCTGTTAATACTCAGCGTGTTGCTCAG;
Amino acid sequence:
(SEQ ID NO: 43156)
AQPVNTQRVAQ,
#5
(SEQ ID NO: 43104)
AQKNGIVDSAQ,
#6
(SEQ ID NO: 43176)
AQGTSLKGPAQ,
(SEQ ID NO: 43089)
AQDATRALAAQ,
and
(SEQ ID NO: 43302)
AQHTTNTEVAQ.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

LENGTHY TABLES
The patent application contains a lengthy table section. A copy of the table is available in electronic form from the USPTO web site (https://seqdata.uspto.gov/?pageRequest=docDetail&DocID=US20220281922A1). An electronic copy of the table will also be available from the USPTO upon request and payment of the fee set forth in 37 CFR 1.19(b)(3).

1. An AAV capsid protein comprising
(i) a parent VP1 amino acid sequence selected from the group consisting of SEQ ID NO: 2 or SEQ ID NO: 3, and (ii) at least one targeting peptide inserted into the parent VP1 amino acid sequence of (i),
wherein the targeting peptide comprises at least 4 contiguous amino acids of any member of a group consisting of SEQ ID NO: 43073-43341.

2. The AAV capsid protein of claim 1, wherein the parent VP1 amino acid sequence comprises at least one of a VP2 region and a VP3 region.

3. The AAV capsid protein of claim 2, wherein the targeting peptide is inserted within the VP2 region of the parent VP1 amino acid sequence.

4. The AAV capsid protein of claim 2, wherein the targeting peptide is inserted within the VP3 region of the parent VP1 amino acid sequence.

5. The AAV capsid protein of claim 1, wherein the targeting peptide is inserted at any amino acid position between amino acids 586-592, inclusive, of the parent VP1 amino acid sequence.

6. The AAV capsid protein of claim 5, wherein the targeting peptide is inserted between amino acids 588-589 of the parent VP1 amino acid sequence.

7. The AAV capsid protein of claim 1, wherein the targeting peptide comprises at least 7 contiguous amino acids of any member of the group consisting of SEQ ID NO: 43073-43341.

8. The AAV capsid protein of claim 7, wherein the at least 7 contiguous amino acids comprise amino acids at positions 3 through 9, inclusive, of any member of the group consisting of SEQ ID NO: 43073-43341.

9. A peptide comprising an amino acid sequence selected from the group consisting of SEQ ID NO: 43073-43341.

10. A peptide comprising an amino acid sequence, said amino sequence comprising at least 4 contiguous amino acids of any member of a group consisting of SEQ ID NO: 43073-43341.

11. The peptide of claim 10, wherein the amino acid sequence comprises at least 7 contiguous amino acids of any member of a group consisting of SEQ ID NO: 43073-43341.

12. The peptide of claim 11, wherein the at least 7 contiguous amino acids comprise amino acids at positions 3 through 9, inclusive, of any member of the group consisting of SEQ ID NO: 43073-43341.

13. A nucleic acid sequence encoding the peptide of any of claims 9-12, selected from the group consisting of SEQ ID NO: 43342-43691.

14. An AAV particle comprising an AAV capsid protein of any of claims 1-8 and a viral genome.

15. The AAV particle of claim 14, wherein the viral genome comprises a nucleic acid sequence encoding a payload.

16. The AAV particle of claim 15, wherein the payload is an RNAi agent.

17. The AAV particle of claim 16, wherein the RNAi agent is selected from the group consisting of dsRNA, siRNA, shRNA, pre-miRNA, pri-miRNA, miRNA, stRNA, lncRNA, piRNA, or snoRNA.

18. The AAV particle of claim 17, wherein the RNAi agent, when expressed, inhibits or suppresses the expression of a gene of interest in a cell, wherein the gene of interest is selected from the group consisting of SOD1, MAPT, APOE, HTT, C9ORF72, TDP-43, APP, BACE, SNCA, ATXN1, ATXN2, ATXN3, ATXN7, SCN1A-SCN5A, or SCN8A-SCN11A.

19. The AAV particle of claim 15, wherein the payload is a polypeptide.

20. The AAV particle of claim 19, wherein the polypeptide is selected from the group consisting of an antibody, aromatic L-amino acid decarboxylase (AADC), survival motor neuron 1 (SMN1), frataxin (FXN), ApoE2, GBA1, GRN, ASPA, CLN2, GLB1, SGSH, NAGLU, IDS, NPC1, or GAN.

21. A pharmaceutical composition comprising the AAV particle of any of claims 14-20 and a pharmaceutically acceptable excipient.

22. A method of treating a disease in a subject by administering the pharmaceutical composition of claim 21 to said subject.

23. The method of claim 22, wherein the disease is selected from the group comprising Huntington's Disease, Amyotrophic Lateral Sclerosis, Friedreich's Ataxia, Parkinson's Disease, Alzheimer's Disease, a tauopathy, or neuropathic pain.
